(19) United States
(12) Patent Application Publication
(10) Pub. No.: US 2026/0042715 A1
(43) Pub. Date: Feb. 12, 2026

(54) FLUORINATION PROCESS

(71) Applicant: Royal College of Surgeons in Ireland, Dublin (IE)

(72) Inventors: Mauro Adamo, Dublin (IE); Francesco Alletto, Dublin (IE)

(73) Assignee: Royal College of Surgeons in Ireland, Dublin (IE)

(21) Appl. No.: 19/099,564

(22) PCT Filed: Jul. 28, 2023
(86) PCT No.: PCT/IB2023/057710

§ 371 Date: Jan. 29, 2025

(30) Foreign Application Priority Data
Aug. 1, 2022 (GB) 2211190.0

(51) Int. Cl. C07B 39/00; A61K 51/04; A61K 51/08; C07B 59/00; C07C 227/16; C07H 21/00; C07K 1/13

(52) U.S. Cl. CPC C07B 39/00; A61K 51/0402; A61K 51/0491; A61K 51/08; C07B 59/005; C07B 59/008; C07C 227/16; C07H 21/00; C07K 1/13; C07B 2200/05

(57) ABSTRACT

The present disclosure relates to processes for the fluorination of molecules. One aspect provides a process for incorporating a fluorine atom into a molecule, said process comprising converting a compound of formula X-SG into a compound of formula X-F, wherein G is an optionally substituted C₁-C₆ alkyl group, an optionally substituted aryl group, or an optionally substituted heteroaryl group, and X is an organic group; and wherein the SG group is attached to a secondary or tertiary carbon atom in the organic group X; said process comprising treating said compound of formula X-SG with (i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium, or chloronium; and (ii) a source of fluoride. Uses of the process in the preparation of various fluorinated molecules as well as uses of certain compounds as intermediates in the processes of the present disclosure are also provided.

### FIELD

The present disclosure relates generally to fluorination reactions, and more particularly, to processes for incorporating a fluorine atom into a molecule. Applications of the process are not limited but are particularly suitable for the introduction of fluorine atoms into small molecules, biomolecules and derivatives thereof, and biomimetic molecules. Said fluorinated molecules find utility, for example, as synthetic precursors, research tools, and diagnostic and/or therapeutic agents. The present disclosure also relates to uses of said processes for the preparation of certain fluorinated molecules, and the use of certain compounds as intermediates for such processes.

### BACKGROUND

The selective introduction of fluorine into molecules is of great importance in the chemical and biomedical sciences and associated industries. It is estimated that around 20% of all marketed small-molecule drugs contain fluorine, with this proportion forecast to increase to 30% by 2030. The substitution of hydrogen with fluorine is a common strategy in medicinal chemistry during drug development due to the small size of the fluorine atom, the polarity of the C—F bond, the greater stability of the C—F bond compared to the C—H bond, the highly electron withdrawing property of the fluorine atom, and thus the potential for improved pharmacological activity, chemical and/or metabolic stability, and/or membrane permeation.

Moreover, the introduction of specific fluorine isotopes into molecules is of considerable interest as such fluorinated molecules can be used as probes or diagnostic reagents for medical imaging applications. Fluorine-19, for instance, is NMR-active such that ¹⁹F-labelled molecules can be imaged by MRI (Magnetic Resonance Imaging) techniques. Similarly, radiolabelled molecules comprising the radioisotope fluorine-18 can be followed by PET (Positron Emission Tomography) imaging and associated technologies. PET imaging in particular is extensively used in the clinic for the diagnosis of a range of diseases and disorders. As well as having clear benefits in medical diagnosis, it is also believed that techniques using such labelled compounds can allow pre-clinical studies to be performed in less time and with less cost. In particular, greater insight into aspects such as pharmacokinetics and pharmacodynamics may be realised by the use of such compounds and techniques.

The applications discussed above for fluorinated molecules are not limited to small-molecules. Clearly, ready access to fluorinated biological molecules would expand the scope of application of imaging applications such as those discussed above. Moreover, therapeutic molecules are not limited to small-molecules, and the use of larger molecules as therapeutics such as nucleic acids, antibodies, and derivatives/mimics thereof continues to grow. To this end, there is a particular need for methodologies that allow the introduction of fluorine into not only small-molecules but also biomolecules.

Applications such as radiolabelling pose particular challenges to the preparation of such molecules. For example, ¹⁸F has a half-life of just 109 minutes. Yet an ¹⁸F labelled molecule to be administered to a subject, for example for PET imaging, must often be prepared by a multi-step synthesis and then purified to a sufficient extent so as to meet safety and regulatory requirements before administration. Further, to ensure safe practices in the preparation of radioactive agents, such compounds are preferably prepared with the minimal amount of handling, and processes amenable to automation may be particularly preferred for this reason. In such cases, not only is there a great need for synthetic methodologies that allow the fast, efficient incorporation of the ¹⁸F into the molecule, but also said incorporation should ideally be performed as late-stage as possible prior to administration. In any case, to be useful any fluorination reaction needs to be compatible with other functional groups present on the molecule to be fluorinated and not require conditions or reagents that would lead to the degradation of said molecule, for example extremes of temperature.

Given these challenges and despite extensive efforts the number of methods by which fluorine can be incorporated into molecules, and in particular biomolecules, is still very limited. Many such methods are too slow to be used for the incorporation of ¹⁸F, and others require harsh conditions. At present, the introduction of ¹⁸F into small-molecules can only be performed if an aromatic group is present. For biomolecules in particular, existing methodologies require long reaction times and/or high temperatures and the typical yield of incorporation of ¹⁸F is less than 50%. Further, molecules such as peptides are typically prepared by automated solid phase synthesis and thus any methodology for fluorination of peptides must be compatible with the conditions of said synthesis while also allowing introduction of fluorine, e.g. ¹⁸F, at as late a stage as possible.

There is therefore a need for a general method for fast and enantioselective fluorination, particularly for the fluorination of biomolecules and aliphatic substrates, e.g. at sp³ carbon atoms. Moreover, there is a need for a general method that enables the ready (and preferably late-stage) incorporation of PET radionuclides into any type of bioactive molecule. The process and uses of the present disclosure seek to address these needs.

### SUMMARY

In a first aspect, the present disclosure provides a process for incorporating a fluorine atom into a molecule, said process comprising converting a compound of Formula (I) into a compound of Formula (II):

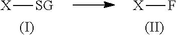

wherein G is an optionally substituted C₁-C₆ alkyl group, an optionally substituted aryl group, or an optionally substituted heteroaryl group, and X is an organic group; and wherein the SG group is attached to a secondary or tertiary carbon atom in the organic group X; said process comprising treating said compound of Formula (I) with:
(i) an activator compound selected from the group consisting of N-halosuccinimides, N-halobenzenesulfonimides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium or chloronium; and
(ii) a source of fluoride.

In a further aspect, the present disclosure provides the use of a process as described herein in the preparation of a fluorinated amino acid or a fluorinated peptide.

In yet another aspect, the present disclosure provides the use of a process as described herein in the preparation of a fluorinated oligonucleotide or polynucleotide.

In a yet further aspect, the present disclosure provides the use of a process as described herein in the preparation of a fluorinated pharmacologically active agent, or a fragment thereof, or a precursor or intermediate thereof.

In another aspect, the present disclosure provides the use of a process as described herein in the preparation of a fluorinated radiolabelling agent.

In a further aspect, the present disclosure provides the use of a compound of Formula (III)

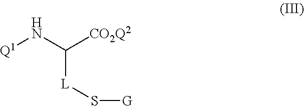

wherein:
Q¹ is H or an amine protecting group;
Q² is H or a carboxyl protecting group;
L is a linker group (CR⁴R⁵), where n is an integer from 1 to 10;
each R⁴ and R⁵ is independently selected from H, alkyl, aryl, COOH, and NH₂; and
G is an optionally substituted C₁-C₆ alkyl group, an optionally substituted aryl group, or an optionally substituted heteroaryl group;
as an intermediate in a process is for preparing fluorinated peptides, wherein said process comprises a step of converting a carbon-SG bond to a carbon-F bond by treating with:
(i) an activator compound selected from the group consisting of N-halosuccinimides, N-halobenzenesulfonimides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with 12; and other sources of Br⁺, Cl⁺, F⁺, I⁺, bromonium, iodonium or chloronium; and
(ii) a source of fluoride.

In a yet further aspect, the present disclosure provides the use of a compound of Formula (IV)

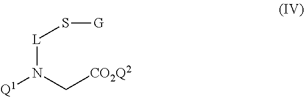

wherein:
Q¹ is H or an amine protecting group;
Q² is H or a carboxyl protecting group;
L is a linker group; and
G is an optionally substituted C₁-C₆ alkyl group, an optionally substituted aryl group, or an optionally substituted heteroaryl group;
as an intermediate in a process is for preparing fluorinated peptoids, wherein said process comprises a step of converting a carbon-SG bond to a carbon-F bond by treating with:
(i) an activator compound selected from the group consisting of N-halosuccinimides, N-halobenzenesulfonimides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium or chloronium; and
(ii) a source of fluoride.

In a further aspect, the present disclosure provides the use of a compound of Formula (V),

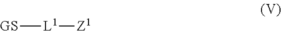

wherein:
Z¹ is a functional group selected from NH₂, N₃, COOH and OH;
L¹ is a linker group (CR⁴R⁵), where n is an integer from 1 to 10;
each R⁴ and R⁵ is independently selected from H, alkyl and aryl; and
G is an optionally substituted C₁-C₆ alkyl group, an optionally substituted aryl group, or an optionally substituted heteroaryl group;
as an intermediate in a process is for preparing a fluorinated oligo- or poly-nucleotide, wherein said process comprises a step of converting a carbon-SG bond to a carbon-F bond by treating with:
(i) an activator compound selected from the group consisting of N-halosuccinimides, N-halobenzenesulfonimides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, bromonium, iodonium or chloronium; and
(ii) a source of fluoride.

In another aspect, the present disclosure provides the use of a compound of Formula (VI),

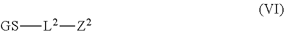

wherein:
Z² is a functional group selected from NH₂, NHR⁶, N₃, COOH, COOR⁷, OH, CN, CONHR⁸, COR⁹, CHO, CSNHR¹⁰, C≡CH, C═CH₂, and

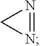

L² is a linker group selected from (CR⁴R⁵)ₙ, an optionally substituted aryl group, an optionally substituted heteroaryl group, an optionally substituted cycloalkyl group, and any combination thereof;
wherein n is an integer from 1 to 10;

each R⁴ and R⁵ is independently selected from H, alkyl and aryl;

R⁶, R⁷, R⁸, R⁹ and R¹⁰ are each independently selected from alkyl, aryl and aralkyl; and
G is an optionally substituted C₁-C₆ alkyl group, an optionally substituted aryl group, or an optionally substituted heteroaryl group;
as an intermediate in a process is for preparing a fluorinated molecule, wherein said process comprises a step of converting a carbon-SG bond to a carbon-F bond by treating with:
(i) an activator compound selected from the group consisting of N-halosuccinimides, N-halobenzenesulfonimides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with 12; and other sources of Br⁺, Cl⁺, F⁺, I⁺, bromonium, iodonium, or chloronium; and
(ii) a source of fluoride.

Further aspects and embodiments of the invention are set out in the appended independent and dependent claims. It will be appreciated that features of the dependent claims may be combined with each other and with features of the independent claims in combinations other than those explicitly set out in the claims. Furthermore, the approaches described herein are not restricted to specific embodiments such as those set out below, but include and contemplate any combinations of features presented herein. The foregoing and other objects, features, and advantages of the present disclosure will appear more fully hereinafter from a consideration of the detailed description that follows.

### DETAILED DESCRIPTION

While various exemplary embodiments are described or suggested herein, other exemplary embodiments utilizing a variety of methods and materials similar or equivalent to those described or suggested herein are encompassed by the general inventive concepts. Aspects and features of apparatus and methods described herein which are not described in detail may be implemented in accordance with any conventional techniques for implementing such aspects and features.

As used in this specification and the claims, the singular forms “a,” “an,” and “the” include plural referents unless the context clearly dictates otherwise.

In this specification, unless otherwise stated, the term “about” modifying the quantity of a component refers to variation in the numerical quantity that can occur, for example, through typical measuring and handling procedures used for making concentrates, mixtures or solutions in the real world; through inadvertent error in these procedures; through differences in the manufacture, source, or purity of the materials employed, or to carry out the methods; and the like. The term “about” also encompasses amounts that differ due to different equilibrium conditions for a composition resulting from a particular initial mixture. Whether or not modified by the term “about”, the claims include equivalents to the quantities.

The ranges provided herein provide exemplary amounts of each of the components. Each of these ranges may be taken alone or combined with one or more other component ranges.

As used herein, the term “at least” includes the end value of the range that is specified. For example, “at least 0° C.” includes the value 0° C.

As used herein, terms have their customary meanings in the art unless otherwise specified.

As used herein, the term “organic group” refers to a group derived by removing a hydrogen atom from an organic compound. An organic compound is a compound that typically comprises a hydrogen atom bonded to a carbon atom, but may also include compounds comprising a covalent bond between carbon and another atom. The term organic compound is well known in the art.

As used herein, the term “hydrocarbyl” refers to a group comprising at least C and H. If the hydrocarbyl group comprises more than one C then those carbons need not necessarily be linked to each other. For example, at least two of the carbons may be linked via a suitable element or group. Thus, the hydrocarbyl group may contain heteroatoms. Suitable heteroatoms will be apparent to those skilled in the art and include, for instance, sulphur, nitrogen, oxygen, phosphorus and silicon. Non-limiting examples of such hydrocarbyls are alkyl groups, such as methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, and isomeric forms thereof; cycloalkyl groups, such as cyclopropyl, cyclobutyl, cyclopentyl, cyclohexyl, cycloheptyl, cycloocytyl, 2-methylcyclopentyl, 2,3-dimethyl-cyclobutyl, 4-methylcyclobutyl, 3-cyclopentylpropyl, and the like; cycloalkenyl groups, such as cyclobutenyl, cyclopentenyl, cyclohexenyl, cycloheptenyl, cyclooctenyl, and the like, and isomeric forms thereof; cycloalkadienyl groups, such as cyclopentadientyl, cyclohexadienyl, cycloheptadienyl, and the like; aryl groups, such as phenyl, tolyl, xylyl, naphthyl, biphenylyl, and the like; aralkyl groups, such as benzyl, phenethyl, phenpropyl, naphthmethyl, and the like. The hydrocarbyl group may be an aryl, heteroaryl, alkyl, cycloalkyl, aralkyl or alkenyl group.

As used herein, the term “alkyl” includes both saturated straight chain and branched alkyl groups which may be substituted (mono- or poly-) or unsubstituted. Preferably, the alkyl group is a C₁₋₂₀ alkyl group, more preferably a C₁₋₁₅, more preferably still a C₁₋₁₂ alkyl group, more preferably still, a C₁₋₆ alkyl group, more preferably a C₁₋₃ alkyl group. Particularly preferred alkyl groups include, for example, methyl, ethyl, propyl, isopropyl, butyl, isobutyl, tert-butyl, pentyl and hexyl. Suitable substituents include, for example, one or more groups selected from OH, O-alkyl, halogen, NH₂, NH-alkyl, N-(alkyl)₂, CF₃, NO₂, CN, COO-alkyl, COOH, CONH₂, CO—NH-alkyl, CO—N(alkyl)₂, SO₂-alkyl, SO₂NH₂ and SO₂—NH-alkyl.

As used herein, the term “aryl” refers to a C₆₋₁₂ aromatic group which may be substituted (mono- or poly-) or unsubstituted. Typical examples include phenyl and naphthyl etc. Suitable substituents include, for example, one or more groups selected from OH, O-alkyl, alkyl, cycloalkyl, halogen, NH₂, NH-alkyl, N-(alkyl)₂, CF₃, NO₂, CN, COO-alkyl, COOH, CONH₂, CO—NH-alkyl, CO—N(alkyl)₂, SO₂-alkyl, SO₂NH₂ and SO₂—NH-alkyl.

The term “aralkyl” is used as a conjunction of the terms alkyl and aryl as given above.

Cyclic alkyl groups, may be referred to as “cycloalkyl” and include those with 3 to 10 carbon atoms having single or multiple fused rings. Non-limiting examples of cycloalkyl groups include adamantyl, cyclopropyl, cyclobutyl, cyclopentyl, cyclohexyl, cycloheptyl, cyclooctyl and the like.

The term “alkenyl” refers to both straight and branched carbon chains which have at least one carbon-carbon double bond. In some embodiments, alkenyl groups may include C₂-C₁₂ alkenyl groups. In other embodiments, alkenyl includes C₂-C₁₀, C₂-C₈, C₂-C₆ or C₂-C₄ alkenyl groups. In one embodiment of alkenyl, the number of double bonds is 1-3; in another embodiment of alkenyl, the number of double bonds is one. Other ranges of carbon-carbon double bonds and carbon numbers are also contemplated depending on the location of the alkenyl moiety on the molecule. “C₂-C₁₀-alkenyl” groups may include more than one double bond in the chain. Examples include, but are not limited to, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

“Heteroaryl” refers to a monovalent aromatic group of from 1 to 15 carbon atoms, preferably from 1 to 10 carbon atoms, having one or more oxygen, nitrogen, and sulfur heteroatoms within the ring, preferably 1 to 4 heteroatoms, or 1 to 3 heteroatoms. The nitrogen and sulfur heteroatoms may optionally be oxidized. Such heteroaryl groups can have a single ring (e.g., pyridyl or furyl) or multiple fused rings provided that the point of attachment is through a heteroaryl ring atom. Examples of heteroaryls include pyridyl, pyridazinyl, pyrimidinyl, pyrazinyl, triazinyl, pyrrolyl, indolyl, quinolinyl, isoquinolinyl, quinazolinyl, quinoxalinnyl, furanyl, thiophenyl, furyl, pyrrolyl, imidazolyl, oxazolyl, isoxazolyl, isothiazolyl, pyrazolyl benzofuranyl, benzothiophenyl, imidazopyridyl, imidazopyrimidyl, or pyrrolopyrimidyl. Heteroaryl rings may be unsubstituted or substituted by one or more moieties as described for aryl above.

“Alkoxy” refers to alkyl-O—, wherein alkyl is as defined above. Examples of C₁-C₆-alkoxy include, but are not limited to, methoxy, ethoxy, OCH₂—C₂H₅, OCH(CH₃)₂, n-butoxy, OCH(CH₃)C₂H₅, OCH₂—CH(CH₃)₂, OC(CH₃) 3, n-pentoxy, 1-methylbutoxy, 2-methylbutoxy, 3-methylbutoxy, 1,1-dimethylpropoxy, 1,2-dimethylpropoxy, 2,2-dimethyl-propoxy, 1-ethylpropoxy, n-hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, 1-ethyl-2-methylpropoxy and the like.

As used herein, “aliphatic group” refers to acyclic or cyclic, saturated or unsaturated carbon groups, but not aromatic groups. For example, aliphatic groups may be alkyl, cycloalkyl, or alkenyl groups as defined herein.

In all aspects of the present disclosure described herein, the disclosure includes, where appropriate all enantiomers and tautomers of the compounds described herein. A person skilled in the art will recognise compounds that possess optical properties (one or more chiral carbon atoms) or tautomeric characteristics. The corresponding enantiomers and/or tautomers may be isolated/prepared by methods known in the art.

Some of the compounds described herein may exist as stereoisomers and/or geometric isomers—e.g. they may possess one or more asymmetric and/or geometric centers and so may exist in two or more stereoisomeric and/or geometric forms. The present disclosure contemplates the use of all the individual stereoisomers and geometric isomers of those compounds, and mixtures thereof. The terms used in the claims encompass these forms.

The present disclosure also includes all suitable isotopic variations of the compounds described herein. An isotopic variation is defined herein as one in which at least one atom is replaced by an atom having the same atomic number but an atomic mass different from the atomic mass usually found in nature. Examples of such isotopes include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations, for example those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. As described in more detail herein, substitution with ¹⁸F is commonly employed to prepare compounds suitable for PET imaging. Isotopic variations as described herein can generally be prepared by conventional procedures using appropriate isotopic variations of suitable reagents.

As used herein, the term “secondary carbon atom” refers to a carbon atom that is attached to two other carbon atoms. Similarly, as used herein, the term “tertiary carbon atom” refers to a carbon atom that is attached to three other carbon atoms.

In all aspects of the present disclosure, the disclosure includes, where appropriate, all enantiomers and tautomers of the compounds disclosed herein. A person skilled in the art will recognise compounds that possess optical properties (one or more chiral carbon atoms) or tautomeric characteristics. The corresponding enantiomers and/or tautomers may be isolated/prepared by methods known in the art.

Some of the compounds disclosed herein may exist as stereoisomers and/or geometric isomers—e.g. they may possess one or more asymmetric and/or geometric centres and so may exist in two or more stereoisomeric and/or geometric forms. The present disclosure contemplates the use of all the individual stereoisomers and geometric isomers of those compounds, and mixtures thereof. The terms used in the claims encompass these forms.

The term “natural” when used in connection with biological materials such as a nucleic acid molecules, amino acids, and polypeptides refers to those which are found in nature and not modified by a human being. Conversely, “non-natural” or “synthetic” when used in connection with biological materials refers to those which are not found in nature and/or have been modified by a human being.

The general inventive concept described herein is centred on providing a process for introducing a fluorine atom into a molecule that advantageously requires the use of mild conditions and reagents such that the process is compatible with the fluorination of, for example, biomolecules and common functional groups typically found therein, and/or achieves high yields, and/or provides fast reaction times, and/or allows the fluorination of molecules for which a viable fluorination methodology did not exist prior to the present disclosure. The process of the present disclosure provides a general synthetic methodology for the preparation of fluorinated molecules but is particularly suitable for the labelling of molecules for imaging applications in research or medicine, e.g. biomolecules and small molecules.

In a first aspect, the present disclosure provides a process for incorporating a fluorine atom into a molecule, said process comprising converting a compound of formula (I) into a compound of formula (II):

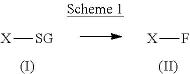

wherein G is an optionally substituted C₁-C₆ alkyl group, an optionally substituted aryl group, or an optionally substituted heteroaryl group, and X is an organic group; and wherein the SG group is attached to a secondary or tertiary carbon atom in the organic group X; said process comprising treating said compound of formula (I) with:
(i) an activator compound selected from the group consisting of N-halosuccinimides, N-imides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium; and
(ii) a source of fluoride.

In another aspect, the present disclosure provides a process for incorporating a fluorine atom into a molecule, said process comprising converting a compound of Formula (I) into a compound of Formula (II):

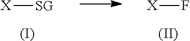

wherein G is an optionally substituted C₁-C₆ alkyl group, an optionally substituted aryl group, or an optionally substituted heteroaryl group, and X is an organic group; and wherein the SG group is attached to a secondary or tertiary carbon atom in the organic group X; said process comprising treating said compound of Formula (I) with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium or chloronium; and
(ii) a source of fluoride.

Any of the following embodiments may be combined with each of the foregoing aspects unless otherwise stated.

In a preferred embodiment, G is a phenyl or heteroaryl group, optionally substituted by one or more substituents independently selected from alkoxy, nitro, halo, and alkyl, or wherein G is selected from methyl and trifluoromethyl.

In another preferred embodiment, G is selected from the group consisting of phenyl, ortho-alkoxyphenyl, para-alkoxyphenyl, and ortho, para-dialkoxyphenyl

In another preferred embodiment G is selected from phenyl, ortho-methoxyphenyl, para-methoxyphenyl, ortho,para-dimethoxyphenyl, and para-nitrophenyl.

In another preferred embodiment G is selected from phenyl, para-methoxyphenyl, and para-nitrophenyl.

In another preferred embodiment, G is selected from phenyl and para-methoxyphenyl.

In a particularly preferred embodiment, G is phenyl. In another particularly preferred embodiment, G is para-methoxyphenyl.

As can be seen from Scheme 1, the process of the present disclosure results in the removal of the —SG group and the concomitant formation of a new C—F bond. A person skilled in the art will also immediately recognise that the process proceeds with a one-to-one replacement of the —SG group with a fluorine atom; i.e. for each —SG group present in the molecule, one new C—F bond will be formed upon removal of the respective —SG group. The number of —SG groups in the molecule is not necessarily limited, however in a preferred embodiment the molecule comprises a single —SG group. Thus, in said embodiment the process of the present disclosure will result in the introduction of a single fluorine atom into the molecule.

The conversion of a compound of Formula (I) into a compound of Formula (II) according to the process of the present disclosure is surprising because as discussed in more detail below, the product of Formula (II) was not expected to be obtained, let alone with high yield and fast reaction times. Moreover, said process has several advantageous characteristics when compared to prior art fluorination reactions. For example, the process of the present disclosure may be chemoselective, i.e. the introduction of the fluorine atom occurs preferentially to replace the —SG group rather than other groups in the molecule. As discussed further below, the process of the present disclosure may also be stereospecific, i.e. the process leads to different stereoisomeric products from different stereoisomeric reactants.

Additionally, the process of the present disclosure may be carried out with mild conditions and reagents, in particular conditions and reagents are compatible with functional groups that are typically present in molecules such as labelling agents, dyes, amino acids, peptides, peptoids, drug molecules or fragments thereof, antibodies or fragments thereof, proteins, carbohydrates e.g. mono-oligo- and poly-saccharides, lipids, nucleobases, nucleosides, nucleotides, oligonucleotides, polynucleotides, peptide nucleic acids, and derivatives thereof. Examples of functional groups present in such molecules that are compatible with the process of the present disclosure include, but are not limited to, amine, hydroxyl, carboxylic acid, aldehyde, azide, tosylate, mesylate and chloride. In particular, due to the mild conditions and reagents of the process of the present disclosure, such functional groups may be present in said molecules during the process of the present disclosure without the need for the use of protecting groups. Protecting groups and strategies for the use thereof in synthesis are well known in the art, as taught in Greene and Wuts, Protective Groups in Organic Synthesis, John Wiley and Sons, Third Edition, 1999. Avoiding the need for protection and deprotection steps can simplify and increase the yield and efficiency of the process, as well as enable the process to be applied to a broader range of starting materials, for example biomolecules. As used herein, the term “biomolecules” refers to biological molecules that are present and/or produced by cells and living organisms.

The introduction of fluorine atoms into molecules is useful for imaging applications. Fluorine-19 is the only stable isotope of fluorine, with an abundance of 100% and a nuclear spin of ½, which a person skilled in the art will understand as meaning that said isotope is NMR-active. ¹⁹F-labeled compounds are thus widely used for Magnetic Resonance Imaging (MRI).

Fluorine-18 is a radioisotope of fluorine which is also useful in imaging applications. For example, fluorination reactions are commonly used for the radiolabelling of compounds with ¹⁸F for use in molecular imaging such as PET (Positron Emission Tomography) scans, as discussed above. Accordingly, in a preferred embodiment of the process of the present disclosure the fluorine atom is ¹⁸F. In particular, the fluorine atom may be ¹⁸F in any of the embodiments disclosed herein. Thus, in a further aspect the present disclosure also provides the use of the process described herein in the preparation of a fluorinated radiolabelling agent.

Due to the short half-life of ¹⁸F (around 109 min) it is desirable that the ¹⁸F radioisotope is introduced into the compound to be labelled as late stage as possible (more preferably as the last stage of the process) prior to purification and administration to a subject, and further that the radiolabelled compound may be purified as quickly and efficiently as possible.

Advantageously, the transformation of the present disclosure proceeds rapidly and results in high yields. For example, the process of the present disclosure may have a reaction time of about 2 minutes to about 5 minutes with isolated yields greater than 90%. Thus, the fast reaction time and high yield of the process of the present disclosure may be particularly advantageous for the introduction of fluorine radioisotopes such as ¹⁸F into molecules as this enables the activity of the radioisotope after preparation of the radiolabelled molecule and prior to administration to be maximised.

In an embodiment of the present disclosure, the steps (i) and (ii) described hereinabove may be performed sequentially, i.e. the process may comprise the sequential steps of:
(a) treating the compound of Formula (I) with the activator as described herein; and
(b) treating the product obtained in step (a) with the source of fluoride as described herein.

For example, a precursor molecule comprising an —SG group may be prepared, such as by the processes described herein, and optionally purified before being supplied to an end-user who may then treat the precursor (e.g. the product obtained in step (a) above) with the source of fluoride as and when required, such as immediately prior to administration to a subject where the fluoride is ¹⁸F for PET imaging. Such a scenario can be possible because the —SG group as defined herein is typically stable and unreactive under storage conditions.

More preferably, the steps (i) and (ii) as defined above may be performed simultaneously. For instance, the activator and source of fluoride may be added to the compound of formula (I) at the same time and/or in the same reaction mixture. This may advantageous to simplify and/or improve one or more of the efficiency, reaction time, and yield of the process and optionally any associated purification steps.

In a preferred embodiment, the process of the present disclosure comprises the step of converting a compound of Formula (IIa) to a compound of Formula (Ia):

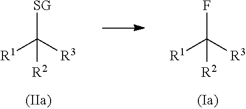

wherein:
R¹ and R² are each independently an optionally substituted hydrocarbyl group;
R³ is H or an optionally substituted hydrocarbyl group;
wherein any two of R¹, R², and R³ or all of R¹, R², and R³ may optionally form one or more cyclic groups.

In a more preferred embodiment, R¹ and R² are each independently an optionally substituted aliphatic group; and R³ is H or an optionally substituted aliphatic group.

Alternatively, R¹ may be an optionally substituted aryl or heteroaryl group; R² may be an optionally substituted aliphatic group; and R³ may be H or an optionally substituted aliphatic group.

A person skilled in the art will recognise that Formulae (IIa) and (Ia) as defined above can encompass embodiments wherein the carbon to which the R¹, R², R³ and SG groups are bonded is a stereogenic centre, i.e. where said carbon is bonded to four different entities (atoms or groups). Thus, such embodiments may encompass different enantiomers and, where further stereogenic centres are present in the molecules, diastereomers.

In one preferred embodiment, the carbon to which the R¹, R², R³ and SG groups are bonded in Formula (IIa) is a stereogenic centre and the conversion of the compound of Formula (IIa) to Formula (Ia) results in at least partial inversion of the stereochemical configuration of the corresponding carbon atom to which F is bonded in Formula (Ia) with respect to that of Formula (IIa). The term “inversion” has its standard meaning as commonly used in the art of the present disclosure. As an example, where a molecule has a stereogenic centre with configuration (R), inversion will result in the configuration of said stereogenic centre being(S) and vice versa.

Where inversion is at least partial, the resulting product may be a mixture of enantiomers in unequal proportions (i.e. at a molar ratio other than 1:1), which may be referred to in the art as a scalemic mixture. For example, the enantiomer having an inverted configuration relevant to the starting enantiomer in the process of the present disclosure may be present in an enantiomeric excess of greater than 0%, greater than 10%, greater than 20%, greater than 30%, greater than 40%, greater than 50%, greater than 60%, greater than 70%, greater than 80%, greater than 90%, greater than 95%, or greater than 99%.

In a more preferable embodiment, the process of the present disclosure results in complete inversion in cases where the carbon to which the R¹, R², R³ and SG groups are bonded in Formula (IIa) is a stereogenic centre. In such cases the product is a single enantiomer, i.e. having an enantiomeric excess of 100%.

For ease of reference, these and further features of the present disclosure are now discussed under appropriate section headings. However, the teachings under each section are not limited to the section in which they are found.

### Activator Compounds

The activator compound of the present disclosure may be selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium or chloronium.

Hypervalent iodine compounds are well known in the chemical arts and are extensively used as reagents, in particular as oxidising agents, in organic synthesis. The skilled person is able to select suitable hypervalent iodine compounds as part as their common general knowledge and the present disclosure is not limited in this respect. The term “hypervalent” takes its usual meaning in the art, namely, it refers to an atom in a molecular entity that has expanded its valence shell beyond the limits of the Lewis octet rule.

Hypervalent iodine compounds of particular use in the present disclosure are hypervalent organoiodine compounds, preferably λ³- and λ⁵-organoiodine compounds (sometimes known as iodanes; wherein λ³ denotes iodine (III) and λ⁵ denotes iodine (V) oxidation states). In preferred embodiments, the hypervalent iodine compounds are selected from iodinanes (e.g. iodosyl/iodoso compounds and their derivatives; iodonium salts) and periodinanes (e.g. iodyl/iodoxy compounds and their derivatives; iodyl salts). None-limiting examples of suitable hypervalent iodine compounds include PIDA (phenyliodine (III) diacetate, also known as (diacetoxyiodo)benzene), PIFA (phenyliodine bis(trifluoroacetate), also known as (bis(trifluoroacetoxy) iodo)benzene), IBX (2-iodoxybenzoic acid) and derivatives thereof, and Dess-Martin Periodinane (DMP; also known as 3-Oxo-1λ^{5,2}-benziodoxole-1,1,1(3H)-triyl triacetate). Such compounds are commercially available. In preferred embodiments, the hypervalent iodine compound is PIDA or PIFA. In a further preferred embodiment, the hypervalent iodine compound is PIDA.

The activator compound of the present disclosure may be selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium. Such activator compounds are commercially available.

As used herein, “bromonium” takes its normal meaning in the art, namely an ion of formula R₂Br⁺, wherein each R is independently H or a hydrocarbyl group, optionally wherein both R groups form a cyclic hydrocarbyl group. Similarly, “chloronium” means an ion of formula R₂Cl⁺, wherein each R is independently a hydrocarbyl group, optionally wherein both R groups form a cyclic hydrocarbyl group. A source of Br⁺, Cl⁺, F⁺, bromonium or chloronium is any molecule or compound capable of providing said Br⁺, Cl⁺, F⁺, bromonium or chloronium ions. In particular, said terms include cases where the Br⁺, Cl⁺, F⁺, bromonium or chloronium ions are formed transiently or only notionally, for example in a formal reaction mechanism as commonly understood in the art of organic chemistry. Such sources of Br⁺, Cl⁺, F⁺, bromonium or chloronium will be known to a person of skill in the art of the present disclosure.

A non-limiting example of a source of Br⁺ is the use of AgNO₃/Br₂. Without wishing to be bound by theory, an exemplary reaction according to the present disclosure using AgNO₃/Br₂ may be considered to proceed via the formation of BrNO₃. Analogously, a nonlimiting example of a source of I⁺ is the use of AgNO₃/I₂.

As used herein “iodonium” means an ion of formula R²I⁺ wherein each R is independently a hydrocarbyl group, optionally wherein both R groups form a cyclic hydrocarbyl group. A source of I⁺ or iodonium is any molecule or compound capable of providing said I⁺ or iodonium ions. In particular, said terms include cases where the I⁺ or iodonium ions are formed transiently or only notionally, for example in a formal reaction mechanism as commonly understood in the art of organic chemistry. Such sources of I⁺ or iodonium will be known to a person of skill in the art of the present disclosure.

A non-limiting example of a source of I⁺ is the use of PIDA and I₂. Without wishing to be bound by theory, an exemplary reaction according to the present disclosure using PIDA with I₂ may be considered to proceed via the in situ formation of CH₃COO—I from PIDA and 12.

In a preferred embodiment, the activator compound is selected from the group consisting of N-bromosuccinimide, N-chlorosuccinimide, N-fluorobenzenesulfonimide, N-chlorobenzenesulfonimide, N-bromobenzenesulfonimide, diethylaminosulfur trifluoride, diethylaminodifluorosulfinium tetrafluoroborate, morpholinodifluorosulfinium tetrafluoroborate, bromine, PIDA/I₂, AgNO₃/I₂, and AgNO₃/Br₂.

In another preferred embodiment, the activator compound of the present disclosure is selected from the group consisting of N-halosuccinimides, N-halobenzenesulfonimides, dialkylaminodihalosulfinium salts, heterocyclylaminodihalosulfinium salts, dialkylaminosulfur trihalides, bromine, and chlorine.

Particularly preferred N-halosuccinimides include N-bromosuccinimide (NBS) and N-chlorosuccinimide (NCS).

Particularly preferred N-halobenzenesulfonimides include N-fluorobenzensulfonimide, N-chlorobenzensulfonimide, and N-bromobenzensulfonimide.

Particularly preferred dialkylaminodihalosulfinium salts include diethylaminosulfur trifluoride, also known as DAST.

Particularly preferred dialkylaminodihalosulfinium salts include diethylaminodifluorosulfinium tetrafluoroborate, which is also known as XtalFluor-E®.

Particularly preferred heterocyclylaminodihalosulfinium salts include morpholinodifluorosulfinium tetrafluoroborate, which is also known as XtalFluor-M®.

Thus, in a preferred embodiment, the activator compound is selected from the group consisting of N-bromosuccinimide, N-chlorosuccinimide, N-fluorobenzenesulfonimide, N-chlorobenzenesulfonimide, N-bromobenzenesulfonimide, diethylaminosulfur trifluoride, diethylaminodifluorosulfinium tetrafluoroborate, morpholinodifluorosulfinium tetrafluoroborate, and bromine.

In a more preferred embodiment, the activator compound is N-bromosuccinimide or N-chlorosuccinimide.

Without wishing to be bound by theory, the activator compound is believed to react with the —SG group of the molecule to form an intermediate adduct. A person skilled in the art will recognise that this reaction step would generally be considered to be an oxidation reaction, i.e. an oxidation of the —SG group. Subsequently, the intermediate adduct is believed to undergo nucleophilic attack by fluoride, wherein the fluoride is provided by the source of fluoride as defined herein. The proposed reaction mechanism is illustrated by the non-limiting example presented in Scheme 2, which exemplifies the proposed reaction mechanism wherein the activator compound is N-bromosuccinimide (NBS).

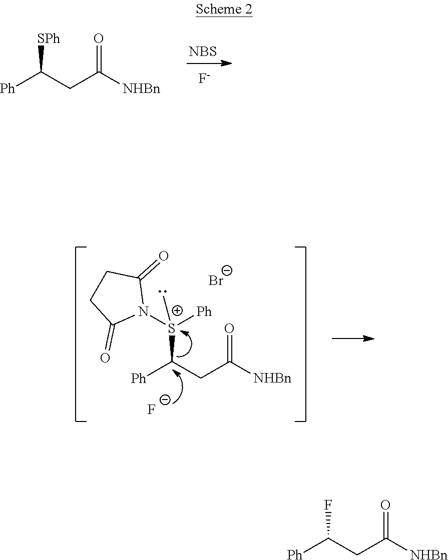

The outcome of the reaction in accordance with the present disclosure as shown in Scheme 2 is entirely surprising because prior to the present disclosure the skilled person would have expected the reaction of such starting materials to result in bromination. Moreover, because fluoride is more basic than bromide the skilled person would have also expected the possibility of gem-disubstituted products via a fluoro-Pummerer type rearrangement, which is promoted by base. The Pummerer rearrangement is a named reaction that will be known to persons skilled in the art as part of their common general knowledge and is discussed in standard textbooks in the art such as March's Advanced Organic Chemistry, 5th Edition, Wiley-Interscience, ISBN 0-471-58589-0.

### Sources of Fluoride

The source of fluoride according to the present disclosure is not limited and can be any substance capable of providing fluoride ions, and the skilled person will be able to select appropriate reagents for supplying fluoride in the process of the present disclosure. For example, the skilled person will be familiar with common fluoride reagents for use in chemical synthesis, and will understand from their common general knowledge which to select for a particular reaction.

It will be understood, in particular from the exemplary embodiments set out below, that the term “source of fluoride” as used herein includes reagents that form fluoride ions transiently or only notionally, for example in a formal reaction mechanism as commonly understood in the art of organic chemistry. In particular, the source of fluoride may be a compound that results in a reaction product that would be expected from e.g. a notional nucleophilic addition of a fluoride ion. Moreover, and while not wishing to be bound by theory, certain reagents are believed to form complex ionic species in aqueous solution. For example, HF in water may form hydronium/fluoride and/or HF₂⁻/hydronium ion pairs and other related species.

In a preferred embodiment, the source of fluoride is selected from the group consisting of metal fluoride salts, complexes or chelates of hydrogen fluoride or metal fluoride salts, and tetraalkylammonium fluorides.

For example, the source of fluoride may be a metal fluoride salt. In a preferred embodiment, the source of fluoride is an alkali metal salt, for example potassium fluoride, sodium fluoride, or cesium fluoride.

As used herein, the term “complex” means a molecular entity formed by the loose association of two or more component entities, typically via one or more coordinate bonds (dative bonds). As used herein, a “chelate” is a type of complex wherein there are coordinate bonds between two or more separate binding sites within the same ligand and a single central atom.

Ionic reactants are often soluble in an aqueous phase but insoluble in an organic phase. Where reactions require a reaction between an ionic reagent and a compound that is more soluble in the organic phase, for example, a source of fluoride that is soluble in organic solvent may be used. In one embodiment, phase transfer catalysts may be used to achieve faster reaction, achieve higher conversion or yields, reduce by-products and/or eliminate the need to use undesirable solvents capable of dissolving all reactants into a single phase. Thus, in a preferred embodiment, the source of fluoride is a fluoride phase transfer catalyst.

In another preferred embodiment, the source of fluoride may be a stabilised form of hydrogen fluoride. For example, the source of fluoride may be a complex of hydrogen fluoride such as pyridine-HF (abbreviations/synonyms of which may include Py-HF, HF-pyridine, PPHF, and Olah's reagent).

Another preferred source of fluoride is triethylamine-HF, also known as triethylamine trihydrofluoride. For instance, in a preferred embodiment the source of fluoride is pyridine-HF or triethylamine-HF.

In another preferred embodiment, the source of fluoride may be a crown ether complex of a metal fluoride, preferably a crown ether complex of an alkali metal fluoride such as potassium fluoride. Crown ethers are polycyclic polyethers with four or more oxygen atoms each separated by two or three carbon atoms having the general formula (OCH₂CH₂)ₙ or (OCH₂CH₂CH₂)ₙ and are well-known in the art of the present disclosure. In particular, crown ethers may be used to increase the solubility of ionic compounds such as metal fluorides in non-aqueous solvents. A person skilled in the art will be able to select suitable crown ether complexes and will understand the nomenclature for referring to said crown ethers as part of their common general knowledge. A non-limiting example of a crown-ether complex of is the complex of KF with 18-crown-6, which is a commercially available crown ether (IUPAC name 1,4,7,10,13,16-hexaoxacyclooctadecane). Other chelates/complexes of metal fluorides are known in the art and contemplated by the present disclosure, for example cryptand complexes of metal fluorides and the like.

In another preferred embodiment, the source of fluoride is a tetraalkylammonium fluoride, preferably a C₁-C₄ tetraalkylammonium fluoride. The alkyl groups of the tetraalkylammonium fluoride are not necessarily limited and include those exemplified herein under the definition of “alkyl”. For example, the C₁-C₄ tetraalkylammonium fluoride may be selected from tetramethylammonium fluoride, tetraethylammonium fluoride, tetrapropylammonium fluoride, tetrabutylammonium fluoride, and (where applicable) structural isomers thereof. In a particularly preferred embodiment, the source of fluoride is tetrabutylammonium fluoride, which is commonly abbreviated as “TBAF”.

Any of the sources of fluoride disclosed herein may be combined with any of the activator compounds disclosed herein. Thus, in one preferred embodiment, the source of fluoride is pyridine-HF and the activator is N-bromosuccinimide. In another preferred embodiment, the source of fluoride is KF and the activator is N-bromosuccinimide. In another preferred embodiment, the source of fluoride is NaF and the activator is N-bromosuccinimide. In another preferred embodiment, the source of fluoride is a crown ether complex of an alkali metal fluoride and the activator is N-bromosuccinimide. In another preferred embodiment, the source of fluoride is tetrabutylammonium fluoride and the activator is N-bromosuccinimide. In another preferred embodiment, the source of fluoride is pyridine-HF and the activator is N-chlorosuccinimide. In another preferred embodiment, the source of fluoride is KF and the activator is N-chlorosuccinimide. In another preferred embodiment, the source of fluoride is NaF and the activator is N-chlorosuccinimide. In another preferred embodiment, the source of fluoride is a crown ether complex of an alkali metal fluoride and the activator is N-chlorosuccinimide. In another preferred embodiment, the source of fluoride is tetrabutylammonium fluoride and the activator is N-chlorosuccinimide.

In another exemplary embodiment, the source of fluoride is triethylamine-HF and the activator is PIDA/I₂.

In another preferred embodiment, the activator compound and the source of fluoride are the same. Contemplated in this embodiment are compounds as disclosed hereinabove as “activator compounds” that comprise a fluorine atom that is able to nucleophilically attack the intermediate formed by the reaction of said compound with a compound of Formula (I) and thereby lead to the formation of a compound of Formula (II). For example, in one particularly preferred embodiment wherein the activator and the source of fluoride is the same, the activator and the source of fluoride are N-fluorobenzenesulfonimide. Without wishing to be bound by theory, the proposed reaction mechanism for the embodiment where the activator and the source of fluoride are N-fluorobenzenesulfonimide (NFSI) is shown in Scheme 3, exemplifying that NFSI can be the activator and the source of fluoride.

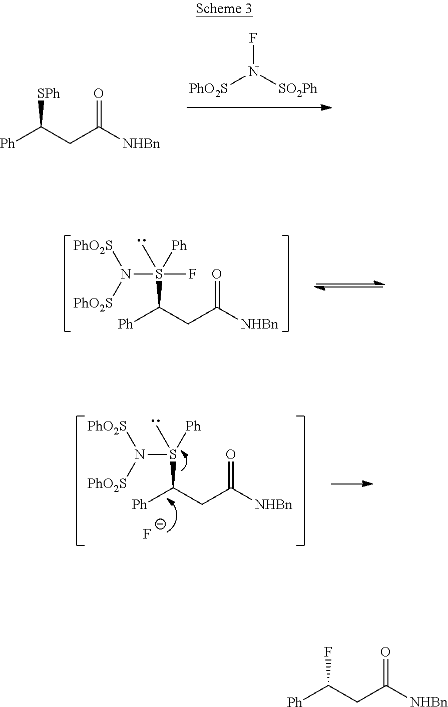

In further examples where the activator and the source of fluoride is the same, the activator and the source of fluoride are XeF₂ or difluoroiodo toluene.

### Reaction Conditions

As discussed herein, the process of the present disclosure may be advantageous in that the reaction conditions are mild, and which in particular allow the introduction of a fluorine atom into molecules that may be sensitive to harsh conditions such as extremes of temperature that lead to degradation of the molecule.

The solvent in which the process of the present disclosure is carried out is not necessarily limited. For example, the process may be carried out in an organic solvent, an aqueous solvent, or combinations thereof. As used herein, an organic solvent has its ordinary meaning in the art, namely a solvent comprising carbon. Non-limiting examples of organic solvents include methanol, ethanol, acetone, n-propanol, n-butanol, isopropyl alcohol, ethyl acetate, dimethyl sulfoxide, sulfuryl chloride, phosphoryl chloride, carbon disulfide, morpholine, N-methylmorpholine, acetonitrile, benzonitrile, dimethylformamide, hydrocarbon oils and blends thereof, toluene, xylene, chloroform, carbon tetrachloride, benzene, hexane, pentane, cyclopentane, cyclohexane, 1,4-dioxane, dichloromethane, nitromethane, propylene carbonate, tetrahydrofuran, diethyl ether, N,N-dimethylformamide, N,N′-dimethylimidazolidin-2-one, N,N-dimethylacetamide, sulfolane, N-methylpyrrolidinone, and methylene chloride.

An aqueous solvent may be water or may comprise water. Where more than one solvent is used, said solvents do not necessarily have to be miscible, for example the solvent may be a biphasic mixture comprising an organic solvent and an aqueous solvent.

In another embodiment, the solvent may be a protic solvent, preferably a polar protic solvent. A person skilled in the art of the present disclosure will be able to identify whether solvents are polar and/or protic, this being part of their common general knowledge.

Aqueous solvents or combinations thereof with one or more organic solvents may be preferred, for example for the fluorination of biomolecules. In a preferred embodiment the process of the present disclosure is carried out in an aqueous solvent.

As used herein, the term “aqueous solvent” may refer to a solvent comprising greater than 50% water.

In a preferred embodiment, the process of the present disclosure is carried out in dichloromethane, tetrahydrofuran, dimethylsulfoxide, acetonitrile, benzonitrile, ethanol, methanol, toluene, water, and combinations thereof.

As discussed herein, the process of the present disclosure may be carried out under mild conditions, for example conditions that maintain the integrity (e.g. to degradation) of the molecule to be fluorinated. In particular, said process may be carried out without the need for high temperatures or harsh reagents while still resulting in the achievement of high yields within a short reaction time. For example, the process may be carried out at a temperature less than about 100° C., less than about 90° C., less than about 80° C., less than about 70° C., less than about 60° C., less than about 50° C., less than about 40° C., or less than about 30° C.

The lower limit of the temperature at which the process can be carried out is not necessarily limited. The process may, for example, be carried out a temperature of at least about 0° C. or at least about 10° C. Thus. in an embodiment the process of the present disclosure is carried out at a temperature of from about 0° C. to about 100° C., preferably from about 0° C. to about 90° C., more preferably from about 0° C. to about 80° C., even more preferably from about 0° C. to about 70° C., even more preferably from about 0° C. to about 60° C., even more preferably from about 0° C. to about 50° C., even more preferably from about 0° C. to about 40° C., even more preferably from about 0° C. to about 30° C. In another embodiment, the process of the present disclosure is carried out at a temperature of from about 10° C. to about 100° C., preferably from about 10° C. to about 90° C., more preferably from about 10° C. to about 80° C., even more preferably from about 10° C. to about 70° C., even more preferably from about 10° C. to about 60° C., even more preferably from about 10° C. to about 50° C., even more preferably from about 10° C. to about 40° C., even more preferably from about 10° C. to about 30° C.

In another preferred embodiment, the process of the present disclosure is carried out room temperature. In an embodiment, room temperature is from about 20° C. to about 25° C.

In a preferred embodiment, the process of the present disclosure is carried out in an aqueous solvent at a temperature of from about 0° C. to about 100° C., preferably from about 0° C. to about 90° C., more preferably from about 0° C. to about 80° C., even more preferably from about 0° C. to about 70° C., even more preferably from about 0° C. to about 60° C., even more preferably from about 0° C. to about 50° C., even more preferably from about 0° C. to about 40° C., even more preferably from about 0° C. to about 30° C.

In a preferred embodiment, the process of the present disclosure is carried out in an aqueous solvent at a temperature of from about 10° C. to about 100° C., preferably from about 10° C. to about 90° C., more preferably from about 10° C. to about 80° C., even more preferably from about 10° C. to about 70° C., even more preferably from about 10° C. to about 60° C., even more preferably from about 10° C. to about 50° C., even more preferably from about 10° C. to about 40° C., even more preferably from about 10° C. to about 30° C.

In an embodiment, the process is carried out in an aqueous solvent at a temperature of from about 10° C. to about 30° C. In an embodiment, the process is carried out in an aqueous solvent at a temperature of from about 20° C. to about 25° C. In another embodiment, the process is carried out in an aqueous solvent at room temperature.

In another embodiment, the process is carried out at a temperature from about 10° C. to about 30° C. in a solvent selected from the group consisting of dichloromethane, tetrahydrofuran, dimethylsulfoxide, acetonitrile, benzonitrile, ethanol, methanol, toluene, water, and combinations thereof. In another embodiment the process is carried out at room temperature in a solvent selected from the group consisting of dichloromethane, tetrahydrofuran, dimethylsulfoxide, acetonitrile, benzonitrile, ethanol, methanol, toluene, water, and combinations thereof. In another embodiment the process is carried out at a temperature of from about 20° C. to about 25° C. in a solvent selected from the group consisting of dichloromethane, tetrahydrofuran, dimethylsulfoxide, acetonitrile, benzonitrile, ethanol, methanol, toluene, water, and combinations thereof. In another embodiment the process is carried out room temperature in a solvent selected from the group consisting of dichloromethane, tetrahydrofuran, dimethylsulfoxide, acetonitrile, benzonitrile, ethanol, methanol, toluene, water, and combinations thereof.

The amount of activator (or as used herein, activator and source of fluoride if the activator and source of fluoride are the same) relative to the compound into which a fluoride atom is to be incorporated is not necessarily limited and the skilled person will be able to select appropriate amounts of compound and activator as a matter of routine practice. For example, the activator may be present in an amount of about 1.0 or greater, about 1.1 or greater, about 1.2 or greater, about 1.5 or greater, or about 2.0 or greater molar equivalents relative to the compound into which a fluorine atom is to be incorporated according to the process of the present disclosure disclosed herein.

In another preferred embodiment, the process is carried out under an inert atmosphere. Inert atmospheres are well known in the art of organic synthesis and the skilled person will be able to select appropriate inert atmospheres as part of their common general knowledge. As used herein, “inert atmosphere” means an atmosphere that is substantially free of reactive gases (e.g. oxygen). For example, the process may be carried out under a nitrogen or argon atmosphere, more preferably a nitrogen atmosphere.

### Applications

The process of the present disclosure is general fluorination method that finds broad applicability. Accordingly, the molecules into which a fluorine atom may be introduced by the process of the present disclosure are not necessarily limited provided that said molecules comprise an —SG group as defined herein. Even where a molecule does not initially comprise an —SG group, the present disclosure encompasses embodiments wherein an —SG group may be incorporated prior to replacement of the —SG group with F as described herein.

Examples of molecules into which it may be desirable to incorporate a fluorine atom by the process of the present disclosure include labelling agents, dyes, amino acids, peptides, peptoids, drug molecules or fragments thereof, antibodies or fragments thereof, proteins, carbohydrates, lipids, nucleobases, nucleosides, nucleotides, oligonucleotides, polynucleotides, peptide nucleic acids, and derivatives thereof.

In such molecules, the —SG group may be joined to said molecules via a linker group. The term “linker group” as used herein refers to any moiety located between the —SG group and the molecule to which it is joined. For example, the linker group may be a hydrocarbyl group. In a preferred embodiment, the linker group may be (CR⁴R⁵), where n is an integer from 1 to 10 and each R⁴ and R⁵ is independently selected from H, alkyl and aryl.

In another preferred embodiment, the linker group may comprise one or more natural or non-natural amino acid residues. In another preferred embodiment the linker group may comprise one or more nucleotides. In another preferred embodiment the linker group may be derived from ethylene glycol or a polyethylene glycol.

The presence of a linker group in embodiments of the present disclosure described herein may be advantageous, for example in higher molecular weight molecules such as proteins where the linker group may aid accessibility of the —SG group, for example to solvent and/or reagents.

Alternatively, the —SG group may be directly bonded to the molecule.

Thus, in one preferred embodiment, the organic group X according to Formula (I) and Formula (II) is of Formula L-Y, where L is a direct bond or a covalent linker group, and Y is selected from the group consisting of a labelling agent, a dye, an amino acid, a peptide, a peptoid, a drug molecule or fragment thereof, an antibody or fragment thereof, a protein, a carbohydrate, a lipid, a nucleobase, a nucleoside, a nucleotide, an oligonucleotide, a polynucleotide, a peptide nucleic acid, and derivatives thereof.

It may be that the molecule into which it is desired to insert a fluorine atom does not initially contain an —SG group. In such cases it may be preferable to first prepare a precursor molecule comprising at least one —SG group as defined herein. Thus, in a preferred embodiment, the process of the present disclosure comprises:
(a) preparing a precursor molecule comprising at least one —SG group, wherein G is an optionally substituted C₁-C₆ alkyl group, an optionally substituted aryl group, or an optionally substituted heteroaryl group;
(b) treating the precursor molecule formed in step (a) with:
   (i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium; and
   (ii) a source of fluoride.

In another preferred embodiment, the process of the present disclosure comprises:
(a) preparing a precursor molecule comprising at least one —SG group, wherein G is an optionally substituted C₁-C₆ alkyl group, an optionally substituted aryl group, or an optionally substituted heteroaryl group;
(b) treating the precursor molecule formed in step (a) with:
   (i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium, or chloronium; and
   (ii) a source of fluoride.

In either of said embodiments, G, the activator compound and/or the source of fluoride may be as further defined hereinabove.

It may be preferable that the —SG group is incorporated into the precursor molecule such that the —SG group is joined thereto via a linker group. Thus, in a preferred embodiment, the —SG group is incorporated into the precursor molecule by reacting a compound of Formula (VI),

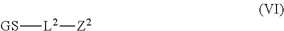

wherein:
Z² is a functional group selected from NH₂, NHR⁶, N₃, COOH, COOR⁷, OH, CN, CONHR⁸, COR⁹, CHO, CSNHR¹⁰;
L² is a linker group (CR⁴R⁵), where n is an integer from 1 to 10;
each R⁴ and R⁵ is independently selected from H, alkyl and aryl;
R⁶, R⁷, R⁸, R⁹, and R¹⁰ are each independently selected from alkyl, aryl and aralkyl; and
G is an optionally substituted C₁-C₆ alkyl group, an optionally substituted aryl group, or an optionally substituted heteroaryl group;
   with a reactive group in the molecule.

In another preferred embodiment, the —SG group is incorporated into the precursor molecule by reacting a compound of Formula (VI),

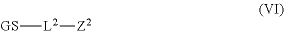

wherein:
Z² is a functional group selected from NH₂, NHR⁶, N₃, COOH, COOR⁷, OH, CN, CONHR⁸, COR⁹, CHO, CSNHR¹⁰, C≡CH, C═CH₂, and

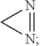

L² is a linker group selected from (CR⁴R⁵) n, an optionally substituted aryl group, an optionally substituted heteroaryl group, an optionally substituted cycloalkyl group, and any combination thereof;
wherein n is an integer from 1 to 10;
each R⁴ and R⁵ is independently selected from H, alkyl and aryl;
R⁶, R⁷, R⁸, R⁹, and R¹⁰ are each independently selected from alkyl, aryl and aralkyl; and
G is an optionally substituted C₁-C₆ alkyl group, an optionally substituted aryl group, or an optionally substituted heteroaryl group;
   with a reactive group in the molecule.

As used herein, the term “reactive group” means a functional group capable of reacting with Z² so as to form a covalent bond such that the —SG group is attached to the molecule via the linker group and any bond or moiety resulting from the reaction of the reactive group with Z². A person skilled in the art will be able to identify and/or select complementary pairs of functional groups capable of reacting with each other in the manner described herein as part of their common general knowledge.

In a preferred embodiment, L² is a linker group selected from (CR⁴R⁵)ₙ, an optionally substituted aryl group, and any combination thereof.

In a preferred embodiment, the —SG group is incorporated into the precursor molecule by reacting a compound of Formula (VIa),

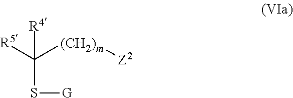

wherein m is an integer from 1 to 10, and
wherein R^{4′} is an aryl group or a tertiary alkyl group and R^{5′} is H, an aryl group or a tertiary alkyl group; or wherein R^{4′} and R^{5′} are both alkyl groups;
   with a reactive group in the molecule.

In a preferred embodiment, the compound of Formula (VIa) is selected from the following:

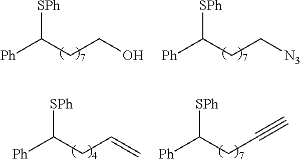

In a preferred embodiment, R^{4′} is phenyl and R^{5′} is H.

In another preferred embodiment, R^{4′} and R^{5′} are both methyl.

In another preferred embodiment, m is 1, 2, or 3.

In a further preferred embodiment, the compound of Formula (VIa) is selected from the following:

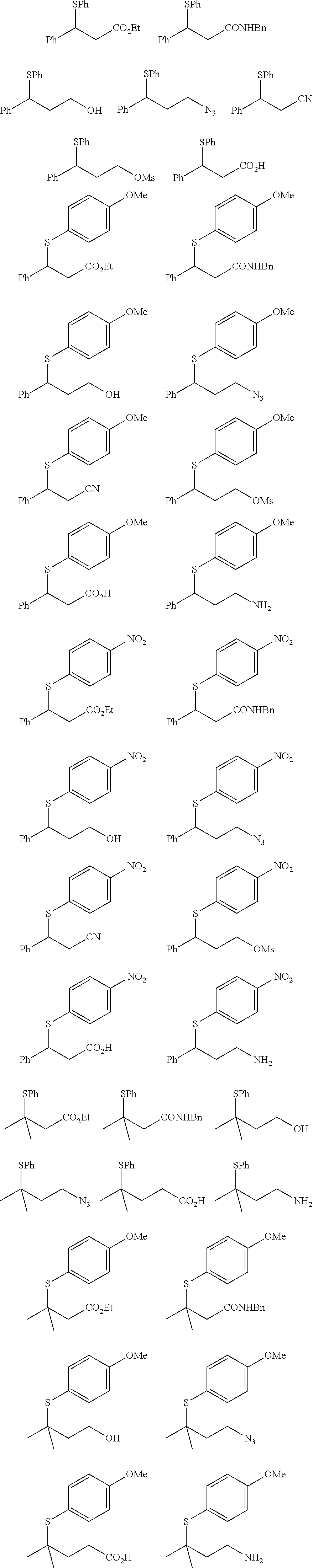

In a further preferred embodiment, the compound of Formula (VIa) is selected from the following:

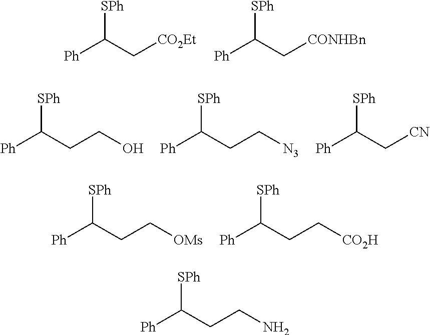

In another preferred embodiment, the compound of Formula (VIa) is selected from the following:

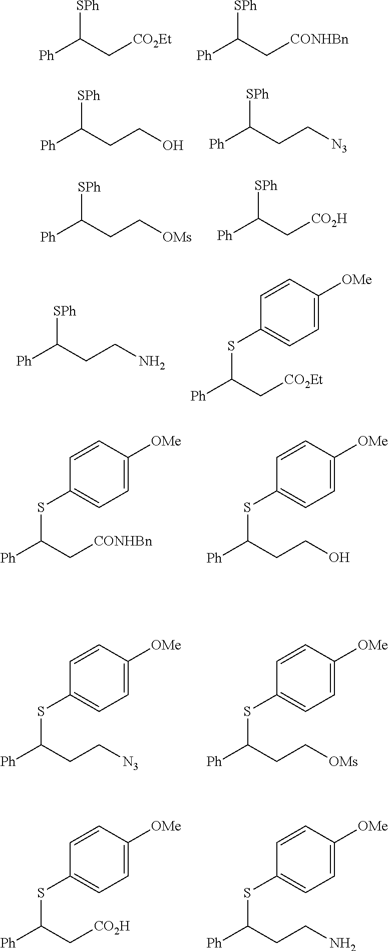

In a further preferred embodiment, the compound of Formula (Via) is selected from the following:

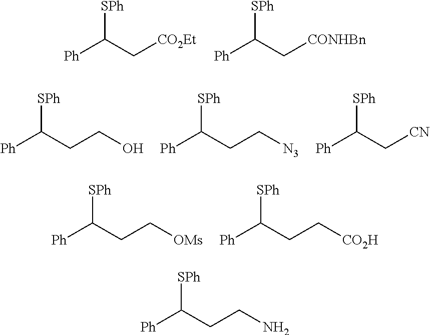

In a further preferred embodiment, the compound of Formula (Via) is selected from the following:

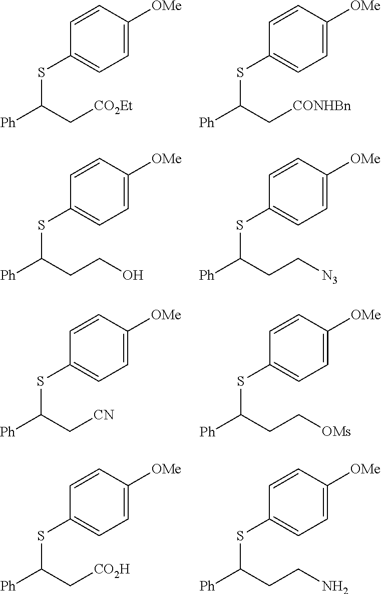

In another preferred embodiment, the compound of Formula (VIa) is selected from the following:

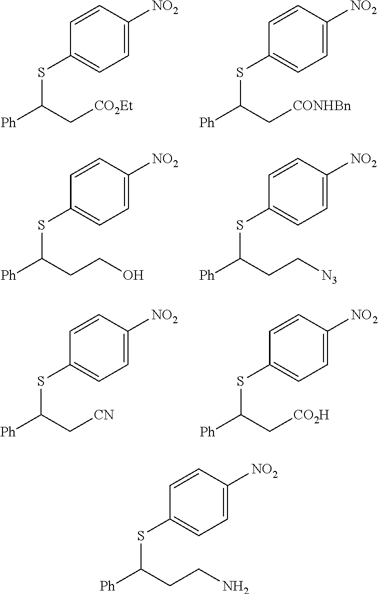

In another preferred embodiment, the compound of Formula (VIa) is selected from the following:

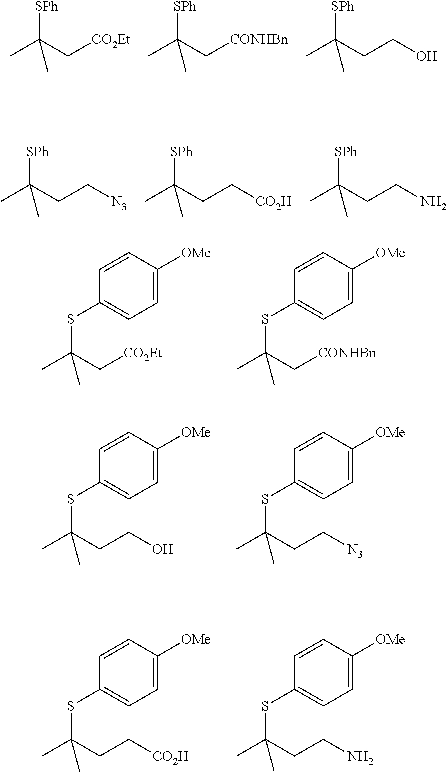

In a preferred embodiment, the —SG group is incorporated into the precursor molecule by reacting a compound of Formula (VIb):

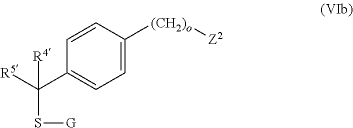

wherein o is 0, 1, 2 or 3, and
wherein R^{4′} is an aryl group or a tertiary alkyl group and R^{5′} is H, an aryl group or a tertiary alkyl group; or wherein R^{4′} and R^{5′} are both alkyl groups;
   with a reactive group in the molecule.

In preferred embodiments, o is 0 or 1.

In another preferred embodiment, the compound of Formula (VIb) is selected from the following:

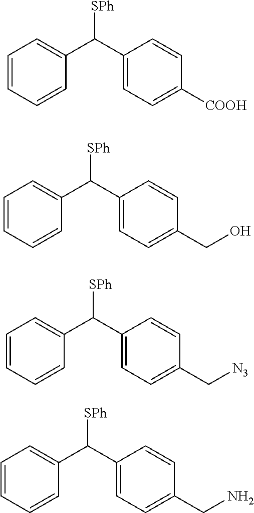

In a further preferred embodiment, the molecule is selected from the group consisting of nucleotides, oligo- or poly-nucleotides, peptide nucleic acids, amino acids, mono-oligo- and poly-saccharides, peptides, peptoids, proteins and small molecules.

The process of the present disclosure may find particular utility in the preparation of fluorinated pharmacological agents. As noted above, the introduction of fluorine, for example as ¹⁹F or ¹⁸F, is of critical importance to drug discovery, development, and production. For example, labelling molecules with ¹⁹F or ¹⁸F enables medical imaging technologies such as MRI and PET imaging for diagnostics or in clinical studies to evaluate properties such as pharmacokinetics and pharmacodynamics. Substitution of a hydrogen atom for a fluorine atom is often a ubiquitous modification made during the development of a drug candidate, such as when evaluating structure activity relationships (SAR). While fluorine is a good bioisostere for hydrogen due to its small size, the substitution of hydrogen with fluorine can also increase biological activity, chemical and/or metabolic stability, and/or membrane permeation. Thus, a further aspect of the present disclosure provides the use of the process described herein in the preparation of a fluorinated pharmacologically active agent, or a fragment thereof, or a precursor or intermediate thereof.

As used herein, “pharmacologically active agent” means any biologically active substance that may be applied or administered to the human or animal body having a diagnostic, therapeutic, or otherwise physiological effect thereon. Such pharmacologically active agents are not limited and may, for example, be small molecules, oligo- or poly-nucleotides, peptides, proteins, or other “biologics” such as antibodies, fragments and derivatives thereof, and antibody mimics such as antigen-binding protein domains. The process described herein may be used to directly incorporate a fluorine atom in such a molecule, or may be used to incorporate a fluorine atom into an intermediate or precursor thereof. For example, the process of the present disclosure may be used to incorporate a fluorine atom into ‘building block’ compounds such as fragments for fragment-based drug discovery processes.

Accordingly, a further aspect of the present disclosure provides the use of a compound of Formula (VI),

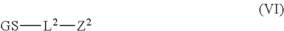

wherein:
Z² is a functional group selected from NH₂, NHR⁶, N₃, COOH, COOR⁷, OH, CN and CONHR⁸, COR⁹, CHO, and CSNHR¹⁰;
L² is a linker group (CR⁴R⁵), where n is an integer from 1 to 10;
each R⁴ and R⁵ is independently selected from H, alkyl and aryl;
R⁶, R⁷, R⁸, R⁹, and R¹⁰ are each independently selected from alkyl, aryl and aralkyl; and
G is an optionally substituted C₁-C₆ alkyl group, an optionally substituted aryl group, or an optionally substituted heteroaryl group;
as an intermediate in a process is for preparing a fluorinated molecule, wherein said process comprises a step of converting a carbon-SG bond to a carbon-F bond by treating with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium; and
(ii) a source of fluoride.

Another aspect provides the use of a compound of Formula (VI),

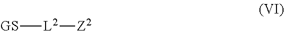

wherein:
Z² is a functional group selected from NH₂, NHR⁶, N₃, COOH, COOR⁷, OH, CN, CONHR⁸, COR⁹, CHO, CSNHR¹⁰, C≡CH, C═CH₂, and

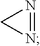

L² is a linker group selected from (CR⁴R⁵) n, an optionally substituted aryl group, an optionally substituted heteroaryl group, an optionally substituted cycloalkyl group, and any combination thereof;
wherein n is an integer from 1 to 10;
each R⁴ and R⁵ is independently selected from H, alkyl and aryl;
R⁶, R⁷, R⁸, R⁹, and R¹⁰ are each independently selected from alkyl, aryl and aralkyl; and
G is an optionally substituted C₁-C₆ alkyl group, an optionally substituted aryl group, or an optionally substituted heteroaryl group;
as an intermediate in a process is for preparing a fluorinated molecule, wherein said process comprises a step of converting a carbon-SG bond to a carbon-F bond by treating with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium, or chloronium; and
(ii) a source of fluoride.

In a further embodiment, the compound of Formula (VI) may be a compound of Formula (VIa) as defined above. In further embodiments, the compound of Formula (VI) may be a compound of Formula (VIb) as defined above.

The molecule is not limited, but in a preferred embodiment may be selected from the group consisting of nucleotides, oligo- or poly-nucleotides, peptide nucleic acids, amino acids, mono-oligo- and poly-saccharides, peptides, peptoids, proteins and small molecules.

Further exemplary embodiments relating to particular types of molecule into which a fluorine atom can be introduced by the process of the present disclosure are now described in more detail below. The sub-headings are for organisational purposes only and the teachings under each section are not limited to the section in which they are found.

### (a) Amino Acids, Peptides and Proteins

The process of the present invention may be particularly advantageous for the introduction of a fluorine atom into an amino acid, peptide or protein.

Amino acids may be natural or non-natural amino acids. Natural amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

As used herein, the term “non-natural amino acid” includes alpha and alpha-disubstituted amino acids, N-alkyl amino acids, lactic acid, halide derivatives of natural amino acids such as trifluorotyrosine, p-Cl-phenylalanine, p-F-phenylalanine, p-Br-phenylalanine, p-NO₂-phenylalanine, phenylglycine, sarcosine, penicillamine, D-2-methyltryptophan, phosphoserine, phosphothreonine, phosphotyrosine, p-I-phenylalanine, L-allyl-glycine, β-alanine, β-aspartic acid, β-cyclohexylalanine, citrulline, homoserine, homocysteine, pyroglutamic acid, L-α-amino butyric acid, L-γ-amino butyric acid, L-α-amino isobutyric acid, α-cyclohexylglycine, diaminobutyric acid, diaminopimelic acid, N-ε-dinitrophenyl-lysine, L-1-naphthylalanine, L-2-naphthylalanine, 3-(2-pyridyl)-L-alanine, 3-(3-pyridyl)-L-alanine, 3-(4-pyridyl)-L-alanine, N-ε-methyl-lysine, N,N-ε-dimethyl-lysine, N,N,N-ε-trimethyl-lysine, 3-mercaptopropionic acid, L-ε-amino caproic acid, 7-amino heptanoic acid, 6-amino hexanoic acid L-methionine sulfone, ornithine, L-norleucine, L-norvaline, p-nitro-L-phenylalanine, L-hydroxyproline, γ-glutamic acid, γ-amino butyric acid L-thioproline, methyl derivatives of phenylalanine (Phe) such as 4-methyl-Phe, pentamethyl-Phe, L-Phe (4-amino), L-Tyr (methyl), L-Phe (4-isopropyl), L-Tic (1,2,3,4-tetrahydroisoquinoline-3-carboxyl acid), L-diaminopropionic acid and L-Phe (4-benzyl).

The compounds described herein may comprise amino acids in the L or D form, i.e. one or more residues, preferably all the residues may be in the L or D form.

One or more amino acids comprised in a peptide may be protected with a protecting group. Suitable protecting groups for amino acids will be familiar to the person skilled in the art (see for example, Chem. Rev. 2009, 109, 2455-2504). These protecting groups can be separated into three groups, as follows:
i. N-terminal protecting groups;
ii. C-terminal protecting groups; and
iii. side chain protecting groups.

Suitable amino protecting groups are described in “Fmoc Solid Phase Peptide Synthesis—A Practical Approach” W. C. Chan & P. D. White. Oxford University Press, 2000, reprinted 2004.

Suitable hydroxy protecting groups are described in Green T., “Protective Groups in Organic Synthesis”, Chapter 1, J. Wiley & Sons, Inc., 1991, 10-142.

Purified, individual amino acids are reacted with these protecting groups prior to synthesis and then selectively removed during specific steps of peptide synthesis.

As used herein, the term “peptide” means a biological oligomer comprising units derived from amino acids linked via peptide bonds. The amino acids may be natural or nonnatural amino acids.

The terms “protein” and “polypeptide” are used interchangeably herein to refer to a biological polymer comprising units derived from amino acids linked via peptide bonds; a protein can be composed of two or more polypeptide chains.

The distinction between peptides and proteins is not precisely defined. However, as an example proteins may be considered to encompass natural or synthetic polypeptides having molecular weights greater than about 10000 Da.

In particular, the reagents and conditions of the process of the present disclosure have good compatibility with functional groups typically present in amino acids, peptides, and proteins such that, for example, protecting groups may not be required. Moreover, the process may be performed in aqueous solvents, which may be particularly compatible with biomolecules such as peptides and proteins, which may not be soluble in organic solvents. Further, existing methods prior to the present disclosure typically require high temperatures which may lead to degradation of the amino acid, peptide or protein.

Existing methods prior to the present disclosure for preparing fluorinated amino acids are generally restricted to the preparation of less reactive amino acids such as 3-fluoroproline. Further, existing methods for preparing ¹⁸F-labelled amino acids for imaging applications are almost entirely limited to derivatives of tyrosine or phenylalanine, because said methods rely on the fluorination of aromatic rings. Moreover, such reactions typically require conditions such as high temperatures that may not be compatible with maintaining the integrity of a peptide or protein, for example.

Thus, a preferred embodiment is a process for incorporating a fluorine into a peptide, said process comprising the steps of:
(a) incorporating at least one compound of Formula (III) into the backbone of a peptide to form a peptide precursor,

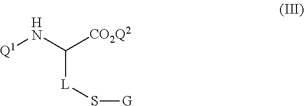

wherein:
Q¹ is H or an amine protecting group;
Q² is H or a carboxyl protecting group;
L is a linker group; and
G is an optionally substituted C₁-C₆ alkyl group, an optionally substituted aryl group, or an optionally substituted heteroaryl group; and
(b) treating the peptide precursor formed in step (a) with:
   (i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium; and
   (ii) a source of fluoride.

In another preferred embodiment of the process of the present disclosure, the process comprises the steps of:
(a) incorporating at least one compound of Formula (III) into the backbone of a peptide to form a peptide precursor,

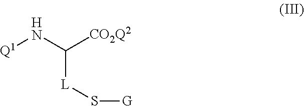

wherein:
Q¹ is H or an amine protecting group;
Q² is H or a carboxyl protecting group;
L is a linker group; and
G is an optionally substituted C₁-C₆ alkyl group, an optionally substituted aryl group, or an optionally substituted heteroaryl group; and
(b) treating the peptide precursor formed in step (a) with:
   (i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium, or chloronium; and
   (ii) a source of fluoride.

Analogous to peptides, the process of the present disclosure may also be applied to the fluorination of peptoids. Peptoids are sequence specific peptidomimetics with a peptide backbone but which differ from peptides in that the side-chains (by analogy to amino acids) are attached to the nitrogen of the peptide backbone rather than to the alpha-carbon as would be the case in an amino acid. The synthesis, characterisation and applications of peptoids are reviewed in Seo et al., 2.3 Peptoids: Synthesis, Characterization, and Nanostructures, Ed: Paul Ducheyne, Comprehensive Biomaterials II, Elsevier, 2017, ISBN: 9780081006924, the disclosure of which is incorporated herein by reference. Such peptidomimetics may be advantageous in certain applications, for example as therapeutic agents, due to their decreased susceptibility to proteolytic degradation. The process of the present disclosure may also be applied to other peptide analogues/mimics such as D-peptides (peptides comprising entirely D-amino acids), beta-peptides (peptides derived from beta-amino acids) and the like.

Thus, in another preferred embodiment the process of the present disclosure is a process for incorporating a fluorine into a peptoid, said process comprising the steps of:
(a) incorporating at least one compound of Formula (IV) into the backbone of a peptoid to form a peptoid precursor,

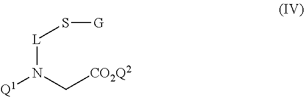

wherein:
Q¹ is H or an amine protecting group;
Q² is H or a carboxyl protecting group;
L is a linker group; and
G is an optionally substituted C₁-C₆ alkyl group, an optionally substituted aryl group, or an optionally substituted heteroaryl group; and
(b) treating the peptoid precursor formed in step (a) with:
   (i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium; and
   (ii) a source of fluoride.

In another preferred embodiment, the process comprises the steps of:
(a) incorporating at least one compound of Formula (IV) into the backbone of a peptoid to form a peptoid precursor,

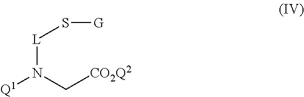

wherein:
Q¹ is H or an amine protecting group;
Q² is H or a carboxyl protecting group;
L is a linker group; and
G is an optionally substituted C₁-C₆ alkyl group, an optionally substituted aryl group, or an optionally substituted heteroaryl group; and
(b) treating the peptoid precursor formed in step (a) with:
   (i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium, or chloronium; and
   (ii) a source of fluoride.

In a preferred embodiment of the above processes, the process comprises simultaneously treating the peptide or peptoid precursor formed in step (a) with said activator and said source of fluoride.

In a preferred embodiment, the fluorine is ¹⁸F.

In a preferred embodiment, G is a phenyl or heteroaryl group, optionally substituted by one or more substituents independently selected from alkoxy, nitro, halo, and alkyl, or wherein G is selected from methyl and trifluoromethyl.

In another preferred embodiment, G is selected from the group consisting of phenyl, ortho-alkoxyphenyl, para-alkoxyphenyl, and ortho, para-dialkoxyphenyl

In another preferred embodiment G is selected from phenyl, ortho-methoxyphenyl, para-methoxyphenyl, ortho,para-dimethoxyphenyl, and para-nitrophenyl.

In another preferred embodiment G is selected from phenyl, para-methoxyphenyl, and para-nitrophenyl.

In another preferred embodiment, G is selected from phenyl and para-methoxyphenyl.

In a particularly preferred embodiment, G is phenyl. In another particularly preferred embodiment, G is para-methoxyphenyl.

In further preferred embodiments, the activator compound and/or source of fluoride may be as further defined in their respective sections hereinabove.

In a preferred embodiment, L is a linker group (CR⁴R⁵), where n is an integer from 1 to 10, and each R⁴ and R⁵ is independently selected from H, alkyl, aryl and COOH, NH₂. In a further preferred embodiment, n is 1 or 2.

In a preferred embodiment, Q¹ is Fmoc (9-fluorenylmethoxycarbonyl) or Boc (t-butyloxycarbonyl).

In a preferred embodiment, Q² is alkyl or aryl, more preferably methyl, ethyl or benzyl.

In a further preferred embodiment, the compound of Formula (III) as defined above is selected from:

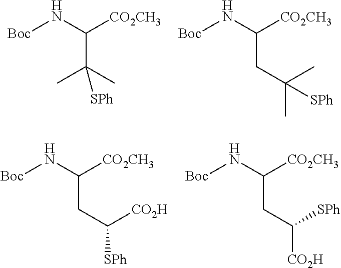

In a preferred embodiment, the peptide or peptoid is prepared by solid phase synthesis. For example, the compound of Formula (III) may be incorporated into the backbone of a peptide during solid phase peptide synthesis. Similarly, the compound of Formula (IV) may be incorporated into the backbone of a peptoid during solid phase synthesis. Incorporation may be at any position within the backbone, and once synthesis is complete the synthesised peptide/peptoid may be cleaved and/or deprotected from the solid phase resin and optionally purified. In another embodiment, the peptide/peptoid comprising the compound of Formula (III) or (IV) may be used in a fragment condensation reaction, in solution or on the solid-phase resin, in which one or more additional peptides/peptoids may be coupled to said peptide/peptoid to produce larger peptides or proteins.

As discussed hereinabove, the process of the present disclosure may be used to incorporate a fluorine atom into a protein. The nature of the protein is not limited, however the process of the present disclosure may be particularly advantageous for the fluorination of certain proteins, for example those that may find therapeutic or diagnostic utility or as tools in research.

In a preferred embodiment, the protein may be an antibody. The term “antibody” means an immunoglobulin molecule (or a group of immunoglobulin molecules) that recognizes and specifically binds to a target, such as a protein, polypeptide, peptide, carbohydrate, polynucleotide, lipid, or combinations of the foregoing through at least one antigen recognition site within the variable region of the immunoglobulin molecule. The terms “antibody” or “immunoglobulin,” as used interchangeably herein, include whole antibodies and antibody fragments including any functional domain of an antibody such as an antigen-binding fragment or single chains thereof, an effector domain, salvage receptor binding epitope, or portion thereof. A typical antibody comprises at least two heavy (H) chains and two light (L) chains interconnected by disulfide bonds. Each heavy chain is comprised of a heavy chain variable region (abbreviated herein as VH) and a heavy chain constant region. The heavy chain constant region is comprised of three domains, CH₁, CH₂, and CH₃. Each light chain is comprised of a light chain variable region (abbreviated herein as VL) and a light chain constant region. The light chain constant region is comprised of one domain, CI. The VH and VL regions can be further subdivided into regions of hypervariability, termed Complementarity Determining Regions (CDRs), interspersed with regions that are more conserved, termed framework regions (FW). Each VH and VL is composed of three CDRs and four FWs, arranged from amino-terminus to carboxy-terminus in the following order: FW1, CDR1, FW2, CDR2, FW3, CDR3, FW4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

Antibodies can include, for example, monoclonal antibodies, recombinantly produced antibodies, human antibodies, humanized antibodies, resurfaced antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), affibodies, Fab fragments, F(ab′)₂ fragments, disulfide-linked Fvs (sdFv), anti-idiotypic (anti-Id) antibodies (including, e.g., anti-anti-Id antibodies), bispecific antibodies, and multi-specific antibodies. In certain embodiments, antibodies described herein refer to polyclonal antibody populations. Antibodies can be of any type (e.g., IgG, IgE, IgM, IgD, IgA, or IgY), any class (e.g., IgG₁, IgG₂, IgG₃, IgG₄, IgA₁, or IgA₂), or any subclasses (isotypes) thereof (e.g. IgG1, IgG2, IgG3, IgG4, IgA1 and IgA2), of immunoglobulin molecule, based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated or fused to other molecules such as toxins, radioisotopes, other polypeptides etc.

The term “antibody fragment” refers to a portion of an intact antibody and refers to any functional domain of an antibody such as an antigen-binding fragment or single chains thereof, an effector domain or a portion thereof, and a salvage receptor binding epitope or a portion thereof. Examples of antibody fragments include, but are not limited to, Fab, Fab′, F(ab′)₂, and Fv fragments, linear antibodies, single chain antibodies, and multi-specific antibodies formed from antibody fragments.

The terms “single chain variable fragment(s),” or “scFv” antibodies as used herein refer to forms of antibodies (e.g., antibody fragments) comprising the variable regions of only the heavy and light chains, connected by a linker peptide.

The term “specifically binds” means that a binding agent such as an antibody or a fragment or derivative thereof reacts or associates more frequently, more rapidly, with greater duration, with greater affinity, or with some combination of the above to the epitope, protein, or target molecule than with alternative substances, including proteins unrelated to the target epitope. Because of the sequence identity between homologous proteins in different species, specific binding can, in several embodiments, include a binding agent that recognizes a protein or target in more than one species. Likewise, because of homology within certain regions of polypeptide sequences of different proteins, specific binding can include a binding agent that recognizes more than one protein or target. It is understood that, in certain embodiments, a binding agent that specifically binds a first target may or may not specifically bind a second target. As such, “specific binding” does not necessarily require (although it can include) exclusive binding, e.g., binding to a single target. Thus, a binding agent may, in certain embodiments, specifically bind more than one target. In certain embodiments, multiple targets may be bound by the same antigen-binding site on the binding agent.

“Target” refers to any molecule or combination of molecules that can be bound by a binding agent such as an antibody or antibody variable domain fragment.

The term “epitope” refers to that portion of any molecule (e.g., a target of interest) capable of being recognized and specifically bound by a particular binding agent (e.g. an antibody or fragment or derivative thereof). When the recognized molecule is a polypeptide, epitopes can be formed from contiguous amino acids and non-contiguous amino acids and/or other chemically active surface groups of molecules (such as carbohydrates) juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3 amino acids, and more usually, at least 5 or 8-10 amino acids in a unique spatial conformation.

In a further aspect, the present disclosure provides the use of the process as described herein in the preparation of a fluorinated amino acid or a fluorinated peptide. In a preferred embodiment of said use, fluorinated amino acid or fluorinated peptide may be radiolabelled, that is a radioisotope of fluorine such as ¹⁸F may be incorporated by the use of the process of the present disclosure.

The present disclosure also contemplates the use of the compounds disclosed herein as intermediates for the preparation of intermediates or precursors for the preparation of fluorinated peptides, peptoids or proteins. Thus, in a further aspect, the present disclosure provides the use of a compound of Formula (III)

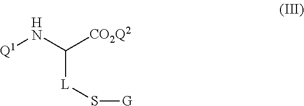

wherein:
Q¹ is H or an amine protecting group;
Q² is H or a carboxyl protecting group;
L is a linker group (CR⁴R⁵), where n is an integer from 1 to 10;
each R⁴ and R⁵ is independently selected from H, alkyl, aryl, COOH, and NH₂; and
G is an optionally substituted C₁-C₆ alkyl group, an optionally substituted aryl group, or an optionally substituted heteroaryl group;
as an intermediate in a process is for preparing fluorinated peptides, wherein said process comprises a step of converting a carbon-SG bond to a carbon-F bond by treating with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br*, Cl⁺, F⁺, bromonium or chloronium; and
(ii) a source of fluoride.

Another aspect provides the use a compound of Formula (III)

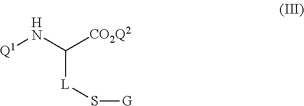

wherein:
   Q¹ is H or an amine protecting group;
   Q² is H or a carboxyl protecting group;
   L is a linker group (CR⁴R⁵), where n is an integer from 1 to 10;
   each R⁴ and R⁵ is independently selected from H, alkyl, aryl, COOH, and NH₂; and
   G is an optionally substituted C₁-C₆ alkyl group, an optionally substituted aryl group, or an optionally substituted heteroaryl group;
as an intermediate in a process is for preparing fluorinated peptides, wherein said process comprises a step of converting a carbon-SG bond to a carbon-F bond by treating with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium, or chloronium; and
(ii) a source of fluoride.

Similarly, in a further aspect the present disclosure provides the use of a compound of Formula (IV)

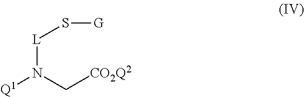

wherein:
Q¹ is H or an amine protecting group;
Q² is H or a carboxyl protecting group;
L is a linker group; and
G is an optionally substituted C₁-C₆ alkyl group, an optionally substituted aryl group, or an optionally substituted heteroaryl group;
as an intermediate in a process is for preparing fluorinated peptoids, wherein said process comprises a step of converting a carbon-SG bond to a carbon-F bond by treating with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium; and
(ii) a source of fluoride.

A further aspect provides the use of a compound of Formula (IV)

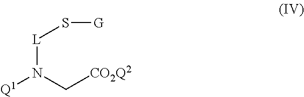

wherein:
Q¹ is H or an amine protecting group;
Q² is H or a carboxyl protecting group;
L is a linker group; and
G is an optionally substituted C₁-C₆ alkyl group, an optionally substituted aryl group, or an optionally substituted heteroaryl group;
   as an intermediate in a process is for preparing fluorinated peptoids, wherein said process comprises a step of converting a carbon-SG bond to a carbon-F bond by treating with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium, or chloronium; and
(ii) a source of fluoride.

In said uses the compound of Formula (III) or Formula (IV) may be as further defined hereinabove.

### (b) Nucleosides, Nucleotides, Oligo- and Poly-Nucleotides

The process of the present invention may be particularly advantageous for the introduction of a fluorine atom into a nucleoside, nucleotide, or oligo- or poly-nucleotide.

The terms “polynucleotide” are used herein interchangeable and refer to a polymeric form of nucleotides of any length, either ribonucleotides or deoxyribonucleotides. These terms include, but are not limited to, DNA, RNA, cDNA (complementary DNA), mRNA (messenger RNA), rRNA (ribosomal RNA), shRNA (small hairpin RNA), snRNA (small nuclear RNA), snoRNA (short nucleolar RNA), miRNA (microRNA), genomic DNA, synthetic DNA, synthetic RNA, and/or tRNA.

Also contemplated within the terms “polynucleotide” and “nucleic acid” are non-natural mimics of polynucleotides as well as natural and non-natural modified polynucleotides. For example, the process of the present invention may be used to incorporate a fluorine atom into a peptide nucleic acid (PNA). PNAs are synthetic DNA analogues in which the phosphodiester backbone is replaced by repetitive units of N-(2-aminoethyl)glycine to which the purine and pyrimidine bases are attached via a methyl carbonyl linker. PNAs may be synthesised in a similar manner to peptides, using standard solid phase synthetic methods.

In another exemplary embodiment, the process of the present invention may be used to incorporate a fluorine atom into a locked nucleic acid (LNA). LNAs are modified nucleotides comprising a 2′-4′ methylene bridge. LNA oligonucleotides typically comprise combinations of LNA and DNA or RNA. LNAs typically have increased binding affinity for complementary nucleic acids, and may also have increased resistance to degradation by nucleases.

Further exemplary modified nucleotides contemplated by the present disclosure are 2′-O-methyl nucleotides and oligonucleotides comprising the same. The 2′-O-methyl nucleotide modification may be natural or non-natural and may be associated with improved nuclease resistance and increased binding affinity for complementary nucleic acids.

Thus, in one preferred embodiment, the process of the present disclosure is a process for incorporating a fluorine atom into an oligo- or poly-nucleotide, the process comprising:
(a) preparing an oligo- or poly-nucleotide precursor comprising at least one —SG group, wherein G is an optionally substituted C₁-C₆ alkyl group, an optionally substituted aryl group, or an optionally substituted heteroaryl group;
(b) treating the oligo- or poly-nucleotide precursor formed in step (a) with:
   (i) an activator compound selected from the group consisting of N-halosuccinimides, N-halobenzenesulfonimides, N-halobenzene sulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium; and
   (ii) a source of fluoride.

In another preferred embodiment, the process of the present disclosure is a process for incorporating a fluorine atom into an oligo- or poly-nucleotide, the process comprising:
(a) preparing an oligo- or poly-nucleotide precursor comprising at least one —SG group, wherein G is an optionally substituted C₁-C₆ alkyl group, an optionally substituted aryl group, or an optionally substituted heteroaryl group;
(b) treating the oligo- or poly-nucleotide precursor formed in step (a) with:
   (i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium, or chloronium; and
   (ii) a source of fluoride.

In a preferred embodiment, the process comprises simultaneously treating the oligo- or poly-nucleotide precursor formed in step (a) with said activator and said source of fluoride.

In a preferred embodiment, the fluorine is ¹⁸F.

In a preferred embodiment, G is a phenyl or heteroaryl group, optionally substituted by one or more substituents independently selected from alkoxy, nitro, halo, and alkyl, or wherein G is selected from methyl and trifluoromethyl.

In another preferred embodiment, G is selected from the group consisting of phenyl, ortho-alkoxyphenyl, para-alkoxyphenyl, and ortho, para-dialkoxyphenyl

In another preferred embodiment G is selected from phenyl, ortho-methoxyphenyl, para-methoxyphenyl, ortho,para-dimethoxyphenyl, and para-nitrophenyl.

In another preferred embodiment G is selected from phenyl, para-methoxyphenyl, and para-nitrophenyl.

In another preferred embodiment, G is selected from phenyl and para-methoxyphenyl.

In a particularly preferred embodiment, G is phenyl. In another particularly preferred embodiment, G is para-methoxyphenyl.

In further preferred embodiments, the activator compound and/or source of fluoride may be as further defined in their respective sections hereinabove.

In another preferred embodiment, the —SG group is attached via a covalent linker group L to the nucleobase of at least one nucleotide in the oligo- or poly-nucleotide.

In another preferred embodiment, the —SG group is incorporated by reacting a compound of Formula (V),

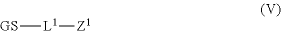

wherein:
Z¹ is a functional group selected from NH₂, N₃, COOH and OH;
L¹ is a linker group (CR⁴R⁵), where n is an integer from 1 to 10;
each R⁴ and R⁵ is independently selected from H, alkyl and aryl; and
G is an optionally substituted C₁-C₆ alkyl group, an optionally substituted aryl group, or an optionally substituted heteroaryl group;
with a reactive group on the nucleobase, wherein a “reactive group” has the meaning as defined hereinabove.

In a further preferred embodiment, n is 1 or 2.

In another preferred embodiment, the —SG group is incorporated by reacting a compound of Formula (Va),

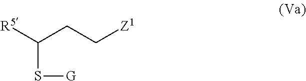

wherein:
Z¹ is a functional group selected from NH₂, N₃, COOH and OH; and
R^{5′} is an aryl group or a tertiary alkyl group;
with a reactive group on the nucleobase.

In a particularly preferred embodiment, Z¹ is N₃ and the reactive group on the nucleobase is a —C≡CH group. In another particularly preferred embodiment, Z¹ is a —C≡CH group and the reactive group on the nucleobase is N₃. In such embodiments, the N₃ group and the C≡CH group undergo a Huisgen 1,3-dipolar cycloaddition resulting in a triazole linkage to the nucleobase. Such a reaction is often referred to in the art as a “click” reaction. Such reactions are commonly used for bioconjugations due to their biocompatibility and bioorthogonality. In particular, such reactions proceed in a single step, are compatible with aqueous solvents, generate minimal byproducts (if any) and have a strong thermodynamic driving force towards quickly and irreversibly producing a high yield of a single reaction product with high specificity. Where Z¹ is N₃ and the reactive group on the nucleobase is a —C≡CH group, the reaction may in a preferred embodiment be catalysed by the presence of Cu(I). Alternatively, the reactive group may be a strained alkyne such as a difluorooctyne or dibenzocyclooctyne.

The nucleobase comprising a reactive group may be incorporated into the oligo- or poly-nucleotide by methods commonplace in the art such as chemical synthesis, enzymatic synthesis, or biosynthesis in a host cell. For example, a natural or modified nucleotide may be incorporated into an oligo-nucleotide during standard phosphoramidite oligonucleotide synthesis. Various alkyne containing nucleotides, precursors and the like are commercially available that are suitable for incorporation into oligo- and poly-nucleotides.

In another preferred embodiment, the compound of Formula (V) is selected from the following:

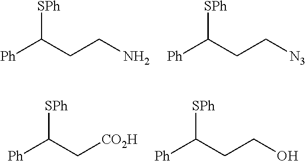

A further aspect of the present disclosure provides the use of the process described herein in the preparation of a fluorinated oligonucleotide or polynucleotide. The fluorinated oligo- or poly-nucleotide may be radiolabelled, for example with ¹⁸F.

In a further aspect the present disclosure provides the use of a compound of Formula (V),

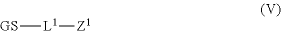

wherein:
Z¹ is a functional group selected from NH₂, N₃, COOH and OH;
L¹ is a linker group (CR⁴R⁵), where n is an integer from 1 to 10;
each R⁴ and R⁵ is independently selected from H, alkyl and aryl; and
G is an optionally substituted C₁-C₆ alkyl group, an optionally substituted aryl group, or an optionally substituted aryl group;
as an intermediate in a process is for preparing a fluorinated oligo- or poly-nucleotide, wherein said process comprises a step of converting a carbon-SG bond to a carbon-F bond by treating with:
   (i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium; and
   (ii) a source of fluoride.

In another aspect, the present disclosure provides the use of a compound of Formula (V),

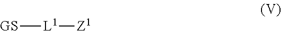

wherein:
Z¹ is a functional group selected from NH₂, N₃, COOH and OH;
L¹ is a linker group (CR⁴R⁵), where n is an integer from 1 to 10;
each R⁴ and R⁵ is independently selected from H, alkyl and aryl; and
G is an optionally substituted C₁-C₆ alkyl group, an optionally substituted aryl group, or an optionally substituted heteroaryl group;
as an intermediate in a process is for preparing a fluorinated oligo- or poly-nucleotide, wherein said process comprises a step of converting a carbon-SG bond to a carbon-F bond by treating with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺,I⁺, bromonium, iodonium, or chloronium; and
(ii) a source of fluoride.

In a preferred embodiment of said uses, the compound of Formula (V) may be as further defined hereinabove. In a further preferred embodiment, the compound of Formula (V) is a compound of Formula (Va) as defined hereinabove.

Having generally described this disclosure, a further understanding can be obtained by reference to certain specific examples illustrated below which are provided for purposes of illustration only and are not intended to be all inclusive or limiting unless otherwise specified.

### EXAMPLES

Exemplary General Procedure for Desulfurative Fluorination with NFSI.

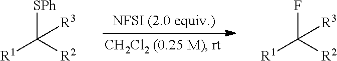

1 (0.25 mmol, 1.0 equiv) was charged in a flame dried 10 mL vial under nitrogen atmosphere and dissolved with 1.0 mL of methylene chloride. Under vigorous stirring, NFSI (0.5 mmol, 2.0 equiv) was added. Upon complete consumption of 1 and/or the relative intermediates (observed via ¹H NMR) the organic phase was concentrated and purified via flash column chromatography on silica gel to isolated desired product 2.

The following exemplary fluorine-containing compounds were each prepared according to Exemplary General Procedure 1.

### (1-fluorobutyl)benzene-2a

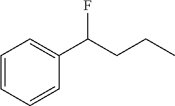

The title compound was isolated by flash column chromatography (silica gel; petroleum ether, 100) as a colourless oil (220 mg, 89% yield). Mild vacuum conditions were used in the concentration process. All analytical data are consistent with those reported in B. Cui, et al., Nat. Commun. 2018, 9, 1-8, the contents of which are incorporated herein by reference.

### Methyl 3-fluoro-3-phenylpropanoate-2b

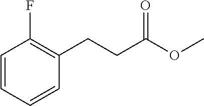

The title compound was isolated by flash column chromatography (silica gel; petroleum ether/EtOAc, 98:2) as a colourless oil (244 mg, 82% yield). All analytical data are consistent with those reported in S. Bloom, et al., Org. Lett. 2013, 15, 7, 1722-1724, the contents of which are incorporated herein by reference.

### Methyl 3-fluoro-3-(o-tolyl) propanoate-2c

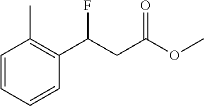

The title compound was isolated by flash column chromatography (silica gel; petroleum ether/EtOAc, 98:2) as a colourless oil (260 mg, 84% yield). ¹H NMR (400 MHz, CDCl₃) δ 7.43-7-37 (m, 1H), 7.27-7.22 (m, 2H), 7.20-7.15 (m, 1H), 6.15 (ddd, J=46.5, 9.5, 3.3 Hz, 1H), 3.75 (s, 3H), 3.05-2.93 (m, 1H), 2.77 (ddd, J=35.1, 16.2, 3.3 Hz, 1H), 2.37 (s, 1H). ¹³C NMR (101 MHz, CDCl₃) δ 170.32 (d, J=2.8 Hz), 136.72 (d, J=18.4 Hz), 134.63 (d, J=4.7), 130.72, 128.69, 126.39, 125.26 (d, J=8.2 Hz), 88.11 (d, J=171.1 Hz), 52.09, 41.23 (d, J=27.3 Hz), 18.88. ¹⁹F NMR (376 MHz, CDCl₃) δ−175.52.

### Ethyl 3-fluoro-3-(2,4,5-trifluorophenyl) propanoate-2d

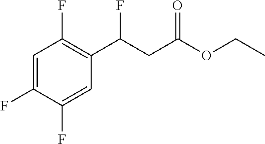 [SMILES: CCOC(=O)CC(F)c1cc(F)c(F)cc1F]

The title compound was isolated by flash column chromatography (silica gel; petroleum ether/EtOAc, 98:2) as a colourless oil (31 mg, 89% yield). ¹H NMR (400 MHz, CDCl₃) δ 7.33-7.25 (m, 1H), 6.96 (dd, J=16.1, 9.6 Hz, 1H), 6.12 (ddd, J=45.7, 8.7, 4.1 Hz, 1H), 4.20 (q, J=7.1 Hz, 1H), 3.03-2.77 (m, 1H), 1.27 (t, J=7.1 Hz, 1H). ¹³C NMR (101 MHz, CDCl₃) δ 168.79 (d, J=4.3 Hz), 154.23 (dddd, J=246.9, 9.3, 5.4, 2.9 Hz), 150.20 (dddd, J=253.1, 14.4, 12.4, 1.9 Hz), 147.09 (ddd, J=246.1, 12.7, 3.6 Hz), 122.92-122.02 (m), 115.34 (ddd, J=14.1, 7.4, 5.3 Hz), 105.93 (dd, J=27.3, 21.1 Hz), 84.51 (dd, J=174.9, 2.0 Hz), 61.20, 41.00 (d, J=26.3 Hz), 14.09. ¹⁹F NMR (376 MHz, CDCl₃) δ−119.65 (dd, J=15.6, 4.4 Hz), −132.44 (ddd, J=21.4, 4.6, 2.6 Hz), −141.35 (ddd, J=21.8, 15.5, 1.3 Hz), −180.25 (d, J=3.9 Hz).

### Methyl 3-(4-chlorophenyl)-3-fluoropropanoate-2e

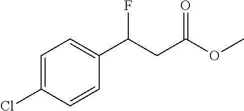

The title compound was isolated by flash column chromatography (silica gel; petroleum ether/EtOAc, 98:2) as a yellow oil (260 mg, 89% yield). ¹H NMR (400 MHz, CDCl₃) δ 7.37 (d, J=8.4 Hz, 2H), 7.31 (d, J=8.4 Hz, 2H), 5.90 (ddd, J=46.6, 8.8, 4.5 Hz, 1H), 3.73 (s, 3H), 3.02 (ddd, J=15.8, 13.8, 8.8 Hz, 1H), 2.78 (ddd, J=31.1, 16.1, 4.4 Hz, 1H). ¹³C NMR (101 MHz, CDCl₃) δ 169.82 (d, J=5.6 Hz), 137.13 (d, J=20.0 Hz), 134.76 (d, J=2.4 Hz), 128.93, 127.03 (d, J=6.5 Hz), 89.90 (d, J=172.9 Hz), 52.11, 42.14 (d, J=27.2 Hz). ¹⁹F NMR (376 MHz, CDCl₃) δ−173.53.

### Methyl 3-fluoro-3-(4-formylphenyl) propanoate-2f

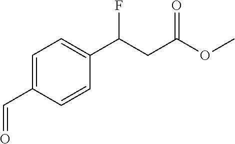

The title compound was isolated as an inseparable mixture with methyl (E)-3-(4-formylphenyl) acrylate (13%) by flash column chromatography (silica gel; petroleum ether/EtOAc, 90:10) as a colourless oil (25 mg, 47% yield based on 2.87f). ¹H NMR (400 MHz, CDCl₃) δ 10.04 (s, 1H), 7.92 (d, J=7.9 Hz, 2H), 7.54 (d, J=8.0 Hz, 2H), 6.01 (ddd, J=46.7, 8.7, 4.3 Hz, 1H), 3.74 (s, 3H), 3.10-2.97 (m, 1H), 2.83 (ddd, J=31.2, 16.2, 4.4 Hz, 1H). ¹³C NMR (101 MHz, CDCl₃) δ 191.62, 169.61 (d, J=5.3 Hz), 145.14 (d, J=19.6 Hz), 136.58 (d, J=1.4 Hz), 130.09, 125.97 (d, J=7.2 Hz), 89.87 (d, J=174.7 Hz), 52.18, 42.14 (d, J=26.5 Hz). ¹⁹F NMR (376 MHz, CDCl₃) δ−177.58.

### 1,1,1,3,3,3-hexafluoropropan-2-yl 3-fluoro-3-phenylpropanoate-2g

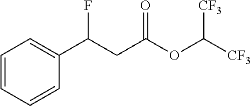 [SMILES: O=C(CC(F)c1ccccc1)OC(C(F)(F)F)C(F)(F)F]

The title compound was isolated by flash column chromatography (silica gel; petroleum ether/EtOAc, 99.5:0.5) as a yellow oil (330 mg, 87% yield). ¹H NMR (400 MHz, CDCl₃) δ 7.39 (q, J=7.2 Hz, 5H), 5.93 (ddd, J=46.9, 9.2, 3.9 Hz, 1H), 5.80 (hept, J=6.0 Hz, 1H), 3.24 (ddd, J=16.3, 12.9, 9.2 Hz, 1H), 3.02 (ddd, J=31.5, 16.3, 3.9 Hz, 1H). ¹³C NMR (101 MHz, CDCl₃) δ 166.52 (d, J=4.4 Hz), 137.52 (d, J=19.4 Hz), 129.30 (d, J=2.0 Hz), 128.86, 125.53 (d, J=6.4 Hz), 120.25 (qd, J=282.0, 3.5 Hz), 89.69 (d, J=174.8 Hz), 66.73 (hept, J=34.9 Hz), 41.45 (d, J=27.6 Hz). ¹⁹F NMR (376 MHz, CDCl₃) δ−73.18-−73.34 (m), −173.32 (s).

### 1,1,1,3,3,3-hexafluoropropan-2-yl 3-(4-chlorophenyl)-3-fluoropropanoate-2h

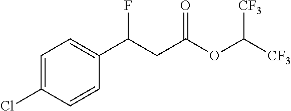 [SMILES: O=C(CC(F)c1ccc(Cl)cc1)OC(C(F)(F)F)C(F)(F)F]

The title compound was isolated as an inseparable mixture with 1,1,1,3,3,3-hexafluoropropan-2-yl (E)-3-(4-chlorophenyl) acrylate (24%) by flash column chromatography (silica gel; petroleum ether/EtOAc, 99.5:0.5) as a yellow oil (390 mg, 84% yield based on 2.87h). ¹H NMR (400 MHz, CDCl₃) δ 7.32 (d, J=7.9 Hz, 2H), 7.24 (d, J=8.2 Hz, 2H), 5.83 (ddd, J=46.6, 9.0, 4.0 Hz, 1H), 5.72 (hept, J=6.1 Hz, 1H), 3.14 (ddd, J=16.1, 13.2, 9.0 Hz, 1H), 2.92 (ddd, J=30.6, 16.4, 4.1 Hz, 1H). ¹³C NMR (101 MHz, CDCl₃) δ 166.27 (d, J=4.8 Hz), 136.02 (d, J=19.9 Hz), 135.30 (d, J=2.3 Hz), 129.14, 126.97 (d, J=6.5 Hz), 120.24 (qd, J=281.9, 3.0 Hz), 89.03 (d, J=175.5 Hz), 66.80 (hept, J=35.0 Hz), 41.34 (d, J=27.6 Hz). ¹⁹F NMR (376 MHz, CDCl₃) δ−73.22-−73.31 (m), −173.65 (s).

### S-phenyl 3-fluoro-3-phenylpropanethioate-2i

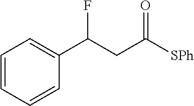

The title compound was isolated by flash column chromatograph (silica gel; petroleum ether/EtOAc, 98:2) as a yellow solid (310 mg, 78% yield). ¹H NMR (400 MHz, CDCl₃) δ 7.45-7.35 (m, 10H), 5.96 (ddd, J=46.8, 8.9, 4.0 Hz, 1H), 3.38 (ddd, J=15.5, 13.5, 8.9 Hz, 1H), 3.07 (ddd, J=31.4, 15.6, 4.0 Hz, 1H). ¹³C NMR (101 MHz, CDCl₃)· 193.63 (d, J=3.7 Hz), 138.4 (d, J=19.6 Hz), 134.50, 129.71, 129.32, 128.96 (d, J=2.0 Hz), 128.74, 127.07, 125.58 (d, J=6.6 Hz), 90.29 (d, J=174.4 Hz), 50.70 (d, J=26.6 Hz). ¹⁹F NMR (376 MHz, CDCl₃) δ−173.88.

### 3-fluoro-3-phenylpropanenitrile-2.I

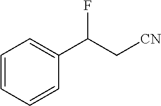 [SMILES: N#CCC(F)c1ccccc1]

The title compound was isolated by flash column chromatography (silica gel; petroleum ether/EtOAc, 95:5) as a colourless oil (310 mg, 88% yield). All analytical data are consistent with those reported in S. Bloom et al., Org. Lett. 2014, 16, 6338-6341.

### 3-(4-bromophenyl)-3-fluoropropyl methanesulphonate-2m

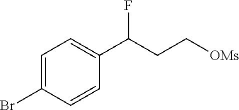 [SMILES: CS(=O)(=O)OCCC(F)c1ccc(Br)cc1]

The title compound was isolated by flash column chromatography (silica gel; petroleum ether/EtOAc, 85:15) as a colourless oil (45 mg, 58% yield). ¹H NMR (400 MHz, CDCl₃) δ 7.53 (d, J=8.0 Hz, 1H), 7.23 (d, J=8.0 Hz, 1H), 5.60 (ddd, J=47.6, 8.9, 3.7 Hz, 1H), 4.44 (td, J=9.4, 5.2 Hz, 1H), 4.34 (dt, J=10.4, 5.3 Hz, 1H), 3.04 (s, 1H), 2.37-2.18 (m, 1H). ¹³C NMR (101 MHz, CDCl₃) δ 137.88 (d, J=19.9 Hz), 131.92 (s), 127.11 (d, J=6.9 Hz), 122.78 (d, J=2.3 Hz), 89.79 (d, J=172.7 Hz), 65.56 (d, J=4.4 Hz), 37.37 (s), 36.77 (d, J=24.0 Hz). ¹⁹F NMR (376 MHz, CDCl₃) δ−179.96.

### 1-chloro-4-(fluoro (phenyl)methyl)benzene-2k

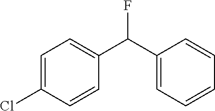 [SMILES: FC(c1ccccc1)c1ccc(Cl)cc1]

The title compound was isolated by flash column chromatography (silica gel; petroleum ether, 100) as a colourless oil (390 mg, 71% yield). All analytical data are consistent with those reported in S. Bloom, et al., Org. Lett. 2014, 16, 6338-6341, the contents of which are incorporated herein by reference.

### 1-fluoroadamantane-2o

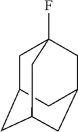 [SMILES: FC12CC3CC(CC(C3)C1)C2]

Prepared according to GP1, 15 minutes reaction. The title compound was isolated by flash column chromatography (silica gel; petroleum ether, 100) as a colourless oil (30 mg, 77% yield). All analytical data are consistent with those reported in H. Chen, et al., Angew. Chemie—Int. Ed. 2017, 56, 15411-15415, the contents of which are incorporated herein by reference.

### (3-fluoro-3-methylbutyl)benzene-2p

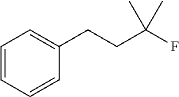

The crude of reaction was analysed with ¹H NMR vs CH₂Br₂ and ¹⁹F NMR vs C₆F₆ to determine the title compound yield (53% yield). All analytical data are consistent with those reported in H. Chen, et al., Angew. Chemie—Int. Ed. 2017, 56, 15411-15415, the contents of which are incorporated herein by reference.

### Exemplary Procedure 2: Desulphurative fluorination of β-sulphido esters with NBS and PPHF

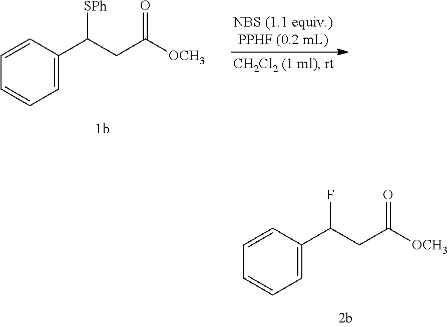

In a plastic vessel flushed with N₂ atmosphere 1b (0.2 mmol, 1.0 equiv) was charged and dissolved with 1 mL of anhydrous CH₂Cl₂. PPHF was added (0.2 mL) followed by addition of NBS (0.24 mmol, 1.1 equiv). The reaction was monitored via ¹H NMR: 0.1 mL of crude solution were rapidly evaporated under high vacuum (P<0.2 mbar), dissolved in 0.4 mL of CDCl₃ and analysed via ¹H NMR. After 10 minutes, ca. 60% conversion to product 2b was detected. Complete consumption of substrate 1b was observed in 20 minutes with 2b present in over 95% yield.

### Exemplary Procedure 3: Desulphurative fluorination of 8-sulphido esters with DAST and PPHF

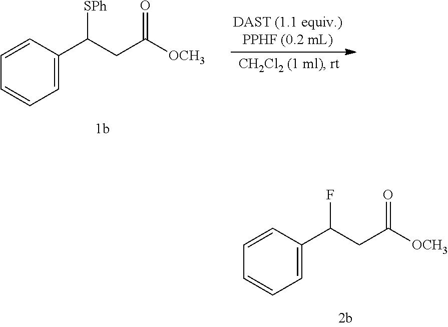

In a plastic vessel flushed with N₂ atmosphere 1b (0.2 mmol, 1.0 equiv) was charged and dissolved with 1 mL of anhydrous CH₂Cl₂. PPHF was added (0.2 mL) followed by addition of DAST (0.24 mmol, 1.2 equiv). The reaction was monitored via ¹H NMR: 0.1 mL of crude solution were rapidly evaporated under high vacuum (P<0.2 mbar), dissolved in 0.4 mL of CDCl₃ and analysed via ¹H NMR. After 10 minutes, ca. 60% conversion to product 1b was detected. Complete consumption of substrate 1b was observed in 15 minutes with 2b as the major (i.e. >98%) product.

### Exemplary Procedure 4: Desulphurative fluorination of 8-sulphido esters with Xtalfluor-E® and PPHF

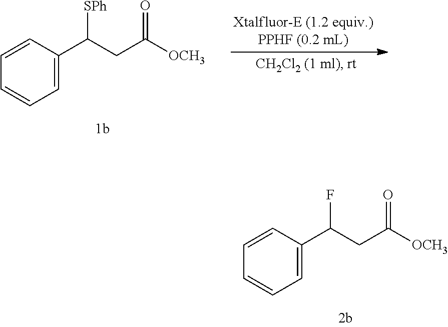

In a plastic vessel flushed with N₂ atmosphere 2a (0.2 mmol, 1.0 equiv) was charged and dissolved with 1 mL of anhydrous CH₂Cl₂. PPHF was added (0.2 mL) followed by addition of Xtalfluor-E® (0.24 mmol, 1.2 equiv). The reaction was monitored via ¹H NMR: 0.1 mL of crude solution were rapidly evaporated under high vacuum (P<0.2 mbar), dissolved in 0.4 mL of CDCl₃ and analysed via ¹H NMR. After 15 minutes, complete consumption of 2a was observed and desired 2b was detected as the sole product.

### Exemplary Procedure 5: Desulphurative fluorination of racemic 8-sulphido esters with difluoroiodo toluene

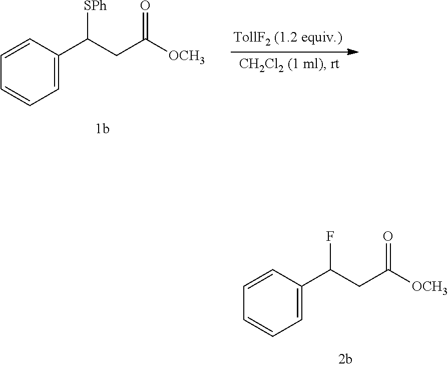

In a flame-dried round flask flushed with N₂ atmosphere 1b (0.2 mmol, 1.0 equiv) was charged and dissolved with 1 mL of deuterated methylene chloride (CD₂Cl₂). Difluoroiodo toluene (0.24 mmol, 1.2 equiv) was then added under vigorous stirring. A gradual colour change was observed: the solution changed from clear faint-yellow to clear amber and reverse in ca. 20 minutes. The reaction was monitored via ¹H NMR: 0.1 mL of crude solution were diluted in 0.3 mL of CDCl₃ and analysed via ¹H NMR. In 30 minutes, the intermediate species were completely consumed with the almost exclusive (>95%) formation of desired product 2b.

### Exemplary Procedure 6: Desulphurative fluorination of racemic 8-sulphido esters with molecular bromine

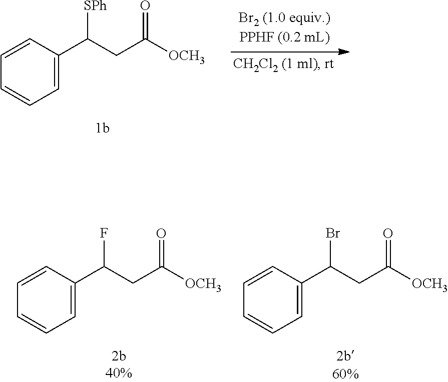

In a plastic vessel flushed with N₂ atmosphere 1b (0.2 mmol, 1.0 equiv) was charged and dissolved with 1 mL of anhydrous CH₂Cl₂. 0.2 mL of PPHF were added followed by dropwise addition of 0.2 mL of a premade 1 M solution of Br₂ in CH₂Cl₂. After 10 minutes at room temperature, the crude solution was rapidly evaporated under high vacuum (P<0.2 mbar), dissolved in 0.4 mL of CDCl₃ and analysed via ¹H NMR. Complete consumption of 1b was observed and a mixture of desired fluorinated 2b and brominated 2b′ was detected in a 37:63 ratio.

### Exemplary Procedure 7: Desulphurative fluorination of racemic B-sulfido esters with PIDA/I₂ and PPHF

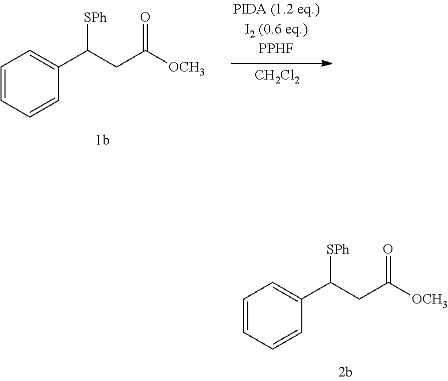

In a plastic vessel, Phl(OAc)₂ (1.2 eq.), I₂ (0.6 eq.) and 1b (1 eq.) were dissolved in CH₂Cl₂ (0.13 M) with vortex stirring for a few seconds until the iodine was completely dissolved. The dark purple solution was stirred for 5 minutes under N₂, and then PPHF (0.1 mL for 1 mmol of starting material) was added by a plastic syringe through a septum. After 5 minutes a reaction aliquot was quenched with water and the organic phase was dried under vacuum. The reaction was performed in the dark. The crude was diluted with CDCl₃, and the product distribution was analysed by ¹H and ¹⁹F NMR. Conversion was 100% after 5 minutes of reaction with 96% yield of the desired product 2b.

### Exemplary Procedure 8: Desulphurative fluorination of racemic B-sulfido esters with PIDA/I₂ and triethylamine-HF

In a plastic vessel, Phl(OAc)₂ (1.2 eq.), 12 (0.6 eq.) and 1b (1 eq.) were dissolved in CH₂Cl₂ (0.13 M) under vortex stirring for few seconds, until the iodine was completely dissolved. The dark purple solution was stirred for 5 minutes under N₂, and then triethylamine-HF (0.1 mL for 1 mmol of starting material) was added by a plastic syringe through a septum. After 5 minutes a reaction aliquot was quenched with water and the organic phase was dried under vacuum. The reaction was performed in the dark. The crude was diluted with CDCl₃, and the product distribution was analysed by ¹H and ¹⁹F NMR.

### Exemplary Procedure 9: Desulphurative fluorination of racemic B-sulfido esters with Br₂, PPHF and AgNO₃

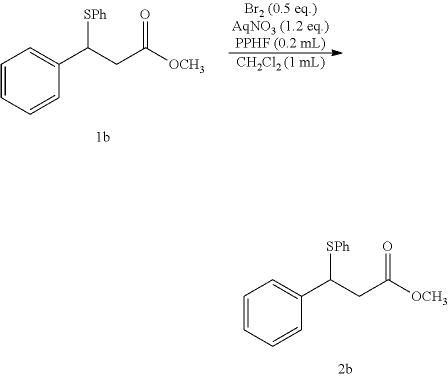

AgNO₃ (1.2 equiv.) and 1 mL of anhydrous CH₂Cl₂ were charged in a plastic vessel flushed with N₂ atmosphere. It was noted that only partial dissolution occurred, and a suspension was obtained. Then, under vigorous stirring (1500 rpm), Br₂ (0.4 mmol, 0.5 equiv.) was added causing an immediate colour change of the organic phase to orange. This was followed by dropwise addition of PPHF (0.2 mL) and vigorous stirring of the resulting mixture for 7 minutes. A gradual color change from brick-orange to yellow and formation of a white precipitate was observed. At this point a solution of methyl 3-phenyl-3-(phenylthio) propanoate 1b (0.18 mmol, 1.0 equiv) in 1 mL of anhydrous CH₂Cl₂ was added dropwise at room temperature and under vigorous stirring. During the reaction time (6 min) the reaction mixture changed its colour from light yellow to cream white. Then, the reaction was quenched by addition of water (4 mL), the biphasic mixture stirred for a further 2 min, the organic layer separated, and the aqueous layer was extracted with additional CH₂Cl₂ (2×10 mL). The combined organic layers were washed with H₂O, brine, dried over Na₂SO₄ and the solvent removed in vacuo to give methyl 3-fluoro-3-phenylpropanoate 2b in 98% isolated yield and high purity as confirmed by ¹H and ¹⁹F NMR.

### Exemplary Procedure 10: Desulphurative fluorination of racemic B-sulfido esters with I₂, PPHF and AgNO₃

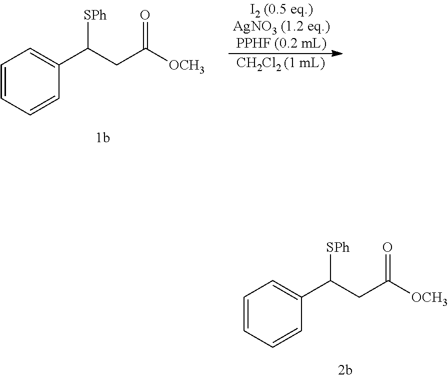

In a plastic vessel flushed with No atmosphere were charged AgNO₃ (1.2 equiv.) and 1 ml of anhydrous CH₂Cl₂. It was noted that only partial dissolution occurred and a suspension was obtained. Then, under vigorous stirring (1500 rpm), iodine (0.5 equiv.) was added, causing an immediate color change of the organic phase to black/violet; this was followed by dropwise addition of PPHF (0.2 mL) and vigorous stirring of the resulting mixture for 10 minutes. No color change and no solid precipitation was observed during this period. At this point a solution of methyl 3-phenyl-3-(phenylthio) propanoate 1b (0.18 mmol, 1.0 equiv.) in 1 mL of anhydrous CH₂Cl₂ was added dropwise at room temperature and under vigorous stirring. A gradual colour change from black/violet to light brown was observed during the reaction. After 40 minutes, the reaction was quenched by addition of water (4 mL), the biphasic mixture stirred for a further 2 min; the organic layer separated and the aqueous layer was extracted with additional CH₂Cl₂ (2×10 mL). The combined organic layers were washed with H₂O, brine, dried over Na₂SO₄ and the solvent removed in vacuo to give methyl 3-fluoro-3-phenylpropanoate 2b in 98% isolated yield and high purity as confirmed by ¹H and ¹⁹F NMR.

Exemplary Preparations of Fluorinable Tags

### 3-phenyl-3-(phenylthio) propanoic acid (3)¹

¹ Gao, S. et. al. Tetrahedron 2006, 47, 1889-1893.

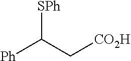

To a mixture of trans-cinnamic acid (740.8 mg, 5.0 mmol) and thiophenol (770 μL, 826.2 mg, 7.5 mmol, 1.5 equiv), iodine (253.8 mg, 1.0 mmol, 20 mol %) was added and the mixture was stirred at room temperature for 15 h. The reaction was quenched by addition of an ice cold saturated solution of sodium thiosulfate (10 mL). The mixture was extracted twice with CH₂Cl₂ (2×15 mL) and the combined organic layers were dried (Na₂SO₄) and concentrated in vacuo. Purification of the residue by flash chromatography on silica gel (Petroleum ether/AcOEt, 5:1 to 100% AcOEt) afforded the pure compound (3) (1.21 g, 4.66 mmol, 93% yield) as an orange solid. ¹H NMR (400 MHz, CDCl₃) δ 7.34-7.25 (m, 10H), 4.64 (t, J=7.6 Hz, 1H), 3.01 (dd, J=7.5, 2.0 Hz, 2H). ¹³C NMR (101 MHz, CDCl₃) δ 176.43, 140.31, 136.66, 133.50, 129.03, 128.67, 128.11, 127.81, 127.77, 48.84, 40.66.

### 3-((4-methoxyphenyl)thio)-3-phenylpropanoic acid (4)

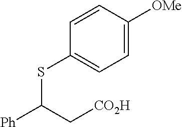 [SMILES: COc1ccc(SC(CC(=O)O)c2ccccc2)cc1]

To a mixture of trans-cinnamic acid (444.5 mg, 3.0 mmol) and 4-methoxythiophenol (553 μL, 630.4 mg, 4.5 mmol, 1.5 equiv), iodine (152.3 mg, 0.6 mmol, 20 mol %) was added and the mixture was stirred at room temperature for 15 h. The reaction was quenched by addition of an ice cold saturated solution of sodium thiosulfate (10 mL). The mixture was extracted twice with CH₂Cl₂ (2×15 mL) and the combined organic layers were dried (Na₂SO₄) and concentrated in vacuo. Purification of the residue by flash chromatography on silica gel (Petroleum ether/AcOEt, 5:1 to 100% AcOEt) afforded the pure compound (4) (769.9 mg, 2.67 mmol, 89% yield) as an orange solid. ¹H NMR (400 MHz, CDCl₃) δ 7.21-7.10 (m, 7H), 6.70 (d, J=8.8 Hz, 2H), 4.41 (t, J=7.7 Hz, 1H), 3.71 (s, 3H), 2.90 (dd, J=7.7, 3.4 Hz, 2H). ¹³C NMR (101 MHz, CDCl₃) δ 176.36, 147.27, 140.54, 136.91, 134.09, 128.58, 127.81, 127.66, 123.49, 114.55, 55.43, 49.71, 40.26.

### Ethyl 3-phenyl-3-(phenylthio) propanoate (5)²

² Canestrari, D. et. al. Org. Lett. 2017, 19, 918-921.

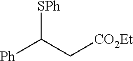

Thiophenol (6.73 mL, 7.22 g, 65.5 mmol, 1.1 equiv) and triethylamine (800 μL, 600.0 mg, 6.0 mmol, 0.1 equiv) were added to the ethyl cinnamate (10.0 mL, 10.5 g, 59.5 mmol) and the reaction mixture was stirred at rt for 24 h. The crude mixture was purified by flash column chromatography on silica gel (Petroleum ether/AcOEt, 95:5) to afford the desired sulfa-Michael compound (16.8 g, 58.5 mmol, 98% yield) as a white solid (5). ¹H NMR (400 MHz, CDCl₃) δ 7.24-7.14 (m, 10H), 4.56 (t, J=7.8 Hz, 1H), 4.01-3.91 (m, 2H), 2.88 (dd, J=14.5, 6.1 Hz, 1H), 2.82 (dd, J=14.6, 7.3 Hz, 1H), 1.06 (t, J=7.1 Hz, 3H). ¹³C NMR (101 MHz, CDCl₃) δ 170.82, 140.67, 133.83, 133.47, 128.95, 128.55, 127.89, 127.80, 127.63, 60.80, 49.31, 41.17, 14.19.

### Ethyl 3-((4-methoxyphenyl)thio)-3-phenylpropanoate (6)

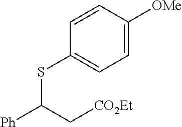 [SMILES: CCOC(=O)CC(Sc1ccc(OC)cc1)c1ccccc1]

4-methoxythiophenol (8.1 mL, 9.18 g, 65.5 mmol, 1.1 equiv) and triethylamine (800 μL, 600.0 mg, 6.0 mmol, 0.1 equiv) were added to the ethyl cinnamate (10.0 mL, 10.5 g, 59.5 mmol) and the reaction mixture was stirred at rt for 24 h. The crude mixture was purified by flash column chromatography on silica gel (Petroleum ether/AcOEt, 95:5) to afford the desired sulfa-Michael compound (16.8 g, 58.5 mmol, 98% yield) as a white solid (6). ¹H NMR (400 MHz, CDCl₃) δ 7.23-7.13 (m, 7H), 6.73 (d, J=8.8 Hz, 2H), 4.45 (t, J=7.8 Hz, 1H), 4.08-3.95 (m, 1H), 3.74 (s, 3H), 2.90 (dd, J=14.5, 6.2 Hz, 1H), 2.85 (dd, J=14.5, 7.1 Hz, 1H), 1.11 (t, J=7.1 Hz, 3H). ¹³C NMR (101 MHz, CDCl₃) δ 170.94, 160.11, 140.82, 136.74, 128.43, 127.81, 127.48, 123.77, 114.46, 60.74, 55.39, 50.14, 40.71, 14.20.

### 3-Phenyl-3-(phenylthio) propan-1-ol (7)³

³ Canestrari, D. et. al. Chem. Sci. 2019, 10, 9042-9050.

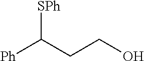

To a solution of sulphide (5) (334.9 mg, 1.2 mmol) in dry THF (2 mL) was added dropwise a solution of LiAlH₄ 1M/THF (585 μL, 0.6 mmol, 0.5 equiv) at 0° C. The reaction mixture was allowed to warm up to room temperature and was stirred for 24 h. The reaction was quenched by addition of a saturated solution of NH₄Cl (7 mL) and the mixture was extracted with Et₂O (3×10 mL). The combined organic layers were dried (Na₂SO₄) and concentrated in vacuo to afford the desired compound (7) (274.6 mg, 1.12 mmol, 96% yield) as a pale yellow oil, which could be used without further purification. ¹H NMR (400 MHz, CDCl₃) δ 7.21-7.11 (m, 10H), 4.28 (t, J=7.6 Hz, 1H), 3.74-3.61 (m, 1H), 3.60-3.46 (m, 1H), 2.22-2.12 (m, 1H), 2.11-2.02 (m, 1H), 1.36 (br s, 1H). ¹³C NMR (101 MHz, CDCl₃) δ 141.86, 134.70, 132.66, 128.84, 128.61, 127.91, 127.40, 127.35, 60.66, 50.36, 38.94.

### 3-((4-methoxyphenyl)thio)-3-phenylpropan-1-ol (8)

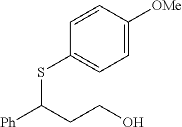 [SMILES: COc1ccc(SC(CCO)c2ccccc2)cc1]

To a solution of sulphide (6) (2.53 g, 8.0 mmol) in dry THF (16 mL) was added dropwise a solution of LiAlH₄ 1M/THF (4 mL, 4 mmol, 0.5 equiv) at 0° C. The reaction mixture was allowed to warm up to room temperature and was stirred for 24 h. The reaction was quenched by addition of a saturated solution of NH₄Cl (40 mL) and the mixture was extracted with Et₂O (3×50 mL). The combined organic layers were dried (Na₂SO₄) and concentrated in vacuo to afford the desired compound (6) (2.19 g, 8.0 mmol, quantitative) as a pale yellow solid, which could be used without further purification. ¹H NMR (400 MHz, CDCl₃) δ 7.20-7.08 (m, 7H), 6.66 (d, J=8.8 Hz, 2H), 4.10 (t, J=7.6 Hz, 1H), 3.75-3.65 (m, 1H), 3.69 (s, 3H), 3.59-3.49 (m, 1H), 2.21-1.99 (m, 2H), 1.34 (br s, 1H). ¹³C NMR (101 MHz, CDCl₃) δ 159.82, 142.01, 136.18, 128.51, 127.98, 127.28, 124.57, 114.39, 60.88, 55.41, 51.62, 38.37.

### 3-phenyl-3-(phenylthio) propyl methanesulfonate (9)³

³ Canestrari, D. et. al. Chem. Sci. 2019, 10, 9042-9050.

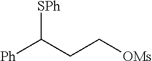

To a stirred solution of 3-phenyl-3-(phenylthio) propan-1-ol (7) (1.62 g, 6.6 mmol) and methanesulfonyl chloride (718 μL, 1.06 g, 9.3 mmol, 1.4 equiv) in dry CH₂Cl₂ (15 mL) was added dropwise triethylamine (1.25 mL, 938 mg, 9.3 mmol, 1.4 equiv) at 0° C. The reaction mixture was allowed to reach rt and stirred overnight, then diluted with H₂O (25 mL) and extracted with CH₂Cl₂ (3×50 mL). The combined organic layers were washed with H₂O (3×50 mL), dried over Na₂SO₄ and concentrated in vacuo. Purification of the residue by flash chromatography on silica gel (Petroleum ether/AcOEt-4:1) afforded the desired compound (9) as a yellow oil (2.08 g, 6.45 mmol, 97% yield). ¹H NMR (400 MHz, CDCl₃) δ 7.10-6.98 (m, 10H), 4.18-4.07 (m, 2H), 3.96-3.90 (m, 1H), 2.69 (s, 3H), 2.25-2.07 (m, 2H). ¹³C NMR (101 MHz, CDCl₃) δ 140.59, 133.86, 133.07, 132.61, 128.98, 128.79, 127.87, 127.80, 67.65, 49.65, 37.33, 35.48.

### 3-((4-methoxyphenyl)thio)-3-phenylpropyl methanesulfonate (10)

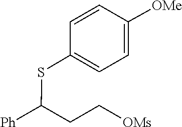 [SMILES: COc1ccc(SC(CCOS(C)(=O)=O)c2ccccc2)cc1]

To a solution of 3-((4-methoxyphenyl)thio)-3-phenylpropan-1-ol (8) (2.2 g, 8.0 mmol) and methanesulfonyl chloride (865 μL, 1.28 g, 11.2 mmol, 1.4 equiv) in dry CH₂Cl₂ (18 mL) was added dropwise triethylamine (1.5 mL, 1.12 g, 11.2 mmol, 1.4 equiv) at 0° C. The reaction mixture was allowed to reach rt and stirred overnight, then diluted with H₂O (25 mL) and extracted with CH₂Cl₂ (3×50 mL). The combined organic layers were washed with H₂O (3×50 mL), dried over Na₂SO₄ and concentrated in vacuo. Purification of the residue by flash chromatography on silica gel (Petroleum ether/AcOEt-4:1) afforded the desired compound (10) as a pale yellow oil (2.33 g, 6.61 mmol, 83% yield).

¹H NMR (400 MHz, CDCl₃) δ 7.30-7.15 (m, 7H), 6.76 (d, J=8.8 Hz, 2H), 4.34 (virt dt, J=10.3, 6.0 Hz, 1H), 4.21-4.07 (m, 2H), 3.78 (s, 3H), 2.90 (s, 3H), 2.44-2.24 (m, 2H). ¹³C NMR (101 MHz, CDCl₃) δ 160.05, 140.75, 136.41, 128.67, 127.91, 127.65, 123.74, 114.50, 67.79, 55.39, 50.60, 37.31, 34.88.

### (3-azido-1-phenylpropyl) (phenyl) sulfane (11)

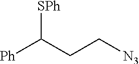

To a solution of (9) (2.08 g, 6.45 mmol) in DMF (20 mL) was added sodium azide (1.05 g, 16.1 mmol, 2.5 equiv) portionwise and the mixture was heated under stirring to 60° C. for 21 h. After cooling to rt, a mixture of EtOH/H₂O (7:3, 30 mL) was added and the mixture was stirred for 1 h at rt. The mixture was extracted with CH₂Cl₂ (3×100 mL) and the combined organic layers were washed with a saturated solution of LiCl (75 mL) and brine (4×75 mL). The resulting organic layer was dried (Na₂SO₄), concentrated in vacuo and the residue was coevaporated with heptane to afford the desired azide (11) (1.59 g, 5.9 mmol, 91% yield) as an orange oil which could be used without further purification. ¹H NMR (400 MHz, CDCl₃) δ 7.22-7.13 (m, 10H), 4.18 (dd, J=8.4, 6.8 Hz, 1H), 3.33 (dt, J=12.5, 6.3 Hz, 1H), 3.14 (dt, J=12.5, 7.2 Hz, 1H), 2.21-2.10 (m, 1H), 2.09-2.01 (m, 1H). ¹³C NMR (101 MHz, CDCl₃) δ 140.99, 134.31, 132.82, 128.94, 128.74, 127.89, 127.65, 127.59, 50.64, 49.29, 35.45.

### (3-azido-1-phenylpropyl) (4-methoxyphenyl) sulfane (12)

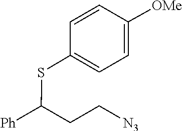 [SMILES: COc1ccc(SC(CCN=[N+]=[N-])c2ccccc2)cc1]

To a solution of (10) (2.33 g, 6.61 mmol) in DMF (20 mL) was added sodium azide (1.07 g, 16.5 mmol, 2.5 equiv) portionwise and the mixture was heated under stirring to 60° C. for 21 h. After cooling to rt, a mixture of EtOH/H₂O (7:3, 30 mL) was added and the mixture was stirred for 1 h at rt. The mixture was extracted with CH₂Cl₂ (3×100 mL) and the combined organic layers were washed with a saturated solution of LiCl (75 mL) and brine (4×75 mL). The resulting organic layer was dried (Na₂SO₄), concentrated in vacuo and the residue was coevaporated with heptane to afford the desired azide (12) (1.84 g, 6.14 mmol, 93% yield) as an orange oil which could be used without further purification. ¹H NMR (400 MHz, CDCl₃) δ 7.21-7.07 (m, 7H), 6.67 (d, J=8.8 Hz, 2H), 4.01 (t, J=7.0 Hz, 1H), 3.69 (s, 3H), 3.33 (dt, J=12.6, 6.4 Hz, 1H), 3.15 (dt, J=12.6, 7.0 Hz, 1H), 2.17-1.99 (m, 2H). ¹³C NMR (101 MHz, CDCl₃) δ 159.95, 141.18, 136.25, 128.63, 127.93, 127.51, 124.22, 114.48, 55.40, 51.77, 49.38, 34.92.

### 3-phenyl-3-(phenylthio) propanenitrile (13)

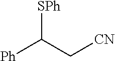

To a mixture of trans-cinnamonitrile (6.3 mL, 6.5 g, 50.0 mmol) and lithium hydroxide (1.2 g, 50.0 mmol, 1.0 equiv) was added thiophenol (5.4 mL, 5.8 g, 52.5 mmol, 1.05 equiv) and the mixture was stirred at room temperature for 5 days. Water was added and the mixture was extracted with CH₂Cl₂ (3×100 mL). The combined organic layers were dried (Na₂SO₄) and concentrated in vacuo. Purification of the residue by flash chromatography on silica gel (Petroleum ether/AcOEt-96:4) afforded the desired compound (13) (8.3 g, 34.6 mmol, 69% yield) as a yellow oil. ¹H NMR (400 MHz, CDCl₃) δ 7.41-7.30 (m, 10H), 4.41 (t, J=7.1 Hz, 1H), 2.90 (d, J=7.6 Hz, 2H). ¹³C NMR (101 MHz, CDCl₃) δ 138.40, 133.94, 132.68, 129.43, 129.10, 128.80, 128.66, 127.58, 117.29, 49.44, 25.24.

### 3-((4-methoxyphenyl)thio)-3-phenylpropanenitrile (14)

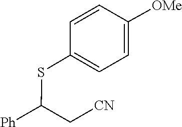 [SMILES: COc1ccc(SC(CC#N)c2ccccc2)cc1]

To a mixture of trans-cinnamonitrile (628 μL, 645.6 mg, 5.0 mmol) and lithium hydroxide (120 mg, 5.0 mmol, 1.0 equiv) was added 4-methoxybenzenethiol (646 μL, 736.4 mg, 5.25 mmol, 1.05 equiv) and the mixture was stirred at room temperature for 5 days. Water was added and the mixture was extracted with CH₂Cl₂ (3×20 mL). The combined organic layers were dried (Na₂SO₄) and concentrated in vacuo. Purification of the residue by flash chromatography on silica gel (Petroleum ether/AcOEt-96:4) afforded the desired compound (14) (560.2 mg, 2.1 mmol, 42% yield) as yellow needles. ¹H NMR (400 MHz, CDCl₃) δ 7.28-7.19 (m, 7H), 6.75 (d, J=8.8 Hz, 2H), 4.19 (t, J=7.2 Hz, 1H), 3.73 (s, 3H), 3.73 (s, 3H), 2.79 (d, J=7.7 Hz, 2H). ¹³C NMR (101 MHz, CDCl₃) δ 160.65, 138.59, 136.87, 128.99, 128.51, 127.58, 122.76, 117.46, 114.94, 55.50, 50.10, 24.81.

### 3-phenyl-3-(phenylthio) propan-1-amine (15)

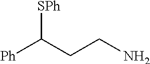

A solution of azide (11) (0.5 mmol) in cyclopentylmethylether (0.25 M) was charged in a round bottomed flask fitted with a magnetic follower and diphenyldisiloxane (DPDS, 1.5 equiv) was then added. To this solution was added PPh₃ (0.1 equiv) and the reaction was left at room temperature for 24 h. Then water was added (10 equiv), the reaction stirred until gas was evolution finished and subsequently dilution with Et₂O and addition of 0.5 ml of 4.0 M HCl in dioxane provided the desired amine (15) as a precipitate (HCl salt).

¹H NMR (400 MHz, CDCl₃) 7.46-7.18 (m, 10H), 4.15 (t, J=8, 1H), 2.59 (t, J=6, 2H), 2.25 (m, 2H), 1.9 (b, 2H); δ ¹³C NMR (101 MHz, CDCl₃) 139.1, 136.8, 136.6, 128.9, 128.8, 128.7, 128.6, 47.7, 41.7, 37.1.

### 3-((4-methoxyphenyl)thio)-3-phenylpropan-1-amine (16)

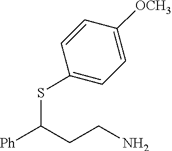 [SMILES: COc1ccc(SC(CCN)c2ccccc2)cc1]

A solution of azide (12) (0.5 mmol) in cyclopentylmethylether (0.25 M) was charged in a round bottomed flask fitted with a magnetic follower and diphenyldisiloxane (DPDS, 1.5 equiv) was then added. To this solution was added PPh₃ (0.1 equiv) and the reaction was left at room temperature for 24 h. Then water was added (10 equiv), the reaction stirred until gas was evolution finished and subsequently dilution with Et₂O and addition of 0.5 ml of 4.0 M HCl in dioxane provided the desired amine (16) as a precipitate (HCl salt). ¹H NMR (400 MHz, CDCl₃) 7.35-7.12 (m, 9H), 4.18 (t, J=8, 1H), 2.58 (t, J=6, 2H), 2.22 (m, 2H), 1.3 (b, 2H); δ ¹³C NMR (101 MHz, CDCl₃) 157.1, 139.5, 136.8, 136.6, 130.9, 128.9, 128.8, 128.1, 125.7, 112.8, 53.9, 41.7, 40.1, 37.4.

### 3-fluoro-3-phenylpropanoic acid (17)

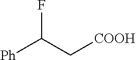 [SMILES: O=C(O)CC(F)c1ccccc1]

Obtained reacting (5) or (6) under the NBS PPHF conditions; yields 79% after column chromatography (Petroleum Ether: AcOEt 90:10 plus 1% AcOH), ¹H NMR (400 MHz, CDCl₃) 12.6 (br s, 1H), 7.53-7.32 (m, 5H), 6.06-5.87 (ddd, ¹H, J=47, 9, 4 Hz), 3.19-3.07 (ddd, ¹H, J=16, 13, 9 Hz), 2.98-2.82 (ddd, ¹H, J=32, 16, 4 Hz); ¹³C NMR (101 MHz, CDCl₃) 175.9, 138.5, 138.2, 129.1, 128.8, 128.6, 128.2, 125.7, 90.4 (d, J=172.5 Hz), 42.3, 42.1; 19F NMR (CDCl₃) δ−172.4 (ddd, 1F, J=46, 33, 1 Hz).

### 3-fluoro-3-phenylpropan-1-ol (18)

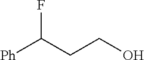 [SMILES: OCCC(F)c1ccccc1]

Obtained reacting (7) or (8) under the NBS PPHF conditions; yields 85% after column chromatography (Petroleum Ether: AcOEt 90:10).

¹H NMR (400 MHz, CDCl₃) 7.47-7.28 (m, 5H, ArH), 5.66 (ddd, J=48, 9, 4 Hz, 1H), 3.94-3.74 (m, 2H), 2.30-1.96 (m, 2H), 1.32 (b, 1H); ¹³C NMR (101 MHz, CDCl₃) 139.9, 128.5, 128.3, 125.7, 92.3 (d, J=169 Hz), 59.1, 39.9, 19F NMR (376 MHz, CDCl₃): 0-177.5.

### (3-azido-1-fluoropropyl)benzene (19)

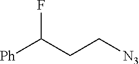 [SMILES: [N-]=[N+]=NCCC(F)c1ccccc1]

Obtained reacting (11) or (12) under the NBS PPHF conditions; yields 92% after column chromatography (Petroleum Ether: AcOEt 90:10). ¹H NMR (400 MHz, CDCl₃) 7.32-7.24 (m, 5H), 5.61 (ddd, J=46, 8, 4 Hz, 1H), 3.29-3.15 (m, 2H), 2.31-2.22 (m, 2H), 1.52-1.39 (2H, m). ¹³C NMR (101 MHz, CDCl₃) 137.8, 128.9, 127.0, 126.1, 93.1 (d, J=164 Hz), 42.1, 32.9.

### Exemplary Preparation of Further Fluorinable Tags

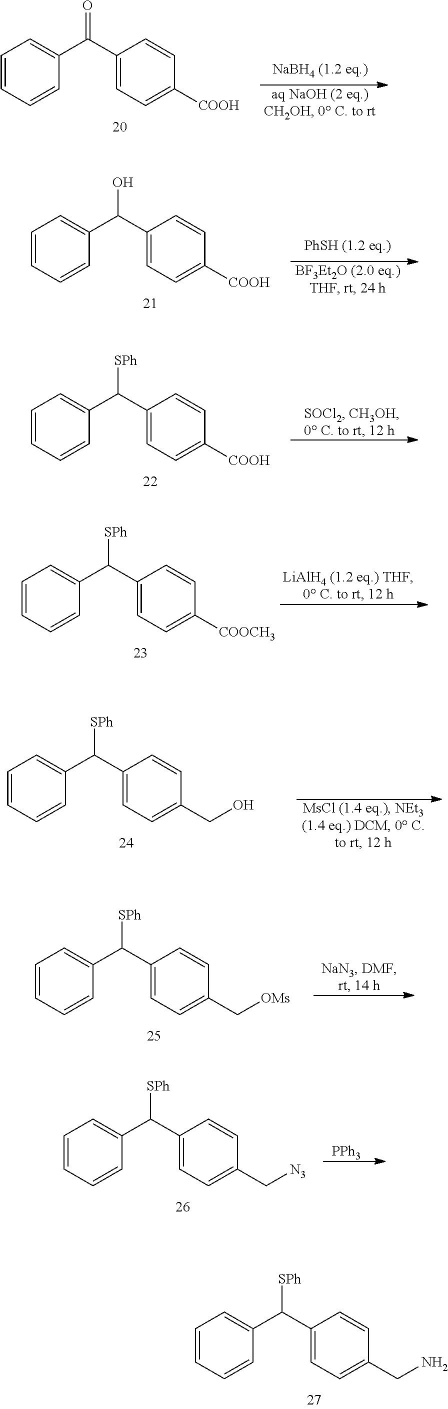

### Preparation of 4-(hydroxy(phenyl)methyl)benzoic acid (21)

To a solution of NaOH (2 mmol) in CH₃OH (2 mL) and water (1 mL) was added starting material 20 (1 mmol). After solid 20 was dissolved, NaBH₄ (1.2 eq.) was added in three portions and the reaction mixture was stirred at room temperature for 14 hrs. Then, the reaction was quenched with conc. HCl until pH 1, and the solid so obtained was filtered, washed with water, and dried under vacuum to give pure 21 which was used further without further purification (162 mg, 71% yield); white solid m.p. 162-1° C. [Kunitscheck, M. J.; Bonner, W. A. J. Org. Chem. 1961, 26, 2194-7]

### Preparation of 4-(phenyl(phenylthio)methyl)benzoic acid (22)

To a stirred solution of acid 21 (2.0 mmol) in dry THF (4.0 mL), thiophenol (1.2 eq.) and BF₃Et₂O (2.0 eq.) were added and the reaction mixture was stirred at room temperature until complete consumption of the starting material was observed by TLC analysis (24 h). The reaction mixture was evaporated and the residue purified by flash column chromatography on silica gel (EtOAc: Pentane 60:40) to afford the title compound as a white solid (486 mg, 76% yield); m.p. 136-4° C.

¹H NMR (400 MHz, CDCl₃) δ 8.03-8.01 (d, J=8.3 Hz, 2H), 7.53-7.51 (d, J=8.3 Hz, 2H), 7.41-7.39 (d, J=7.4 Hz, 3H), 7.33-7.30 (t, J=7.4 Hz, 2H), 7.27-7.21 (br, 3H), 7.20-7.16 (br, 3H), 5.57 (s, 1H).

¹³C NMR (101 MHz, CDCl₃) δ 171.30, 147.29, 140.06, 136.29, 131.03, 132.52, 128.90, 128.76, 128.62, 128.37, 128.09, 127.63, 127.08, 57.48.

### Preparation of methyl 4-(phenyl(phenylthio)methyl)benzoate (23)

To a stirred solution of acid 22 (2.0 mmol) in dry MeOH (4.0 mL) was added dropwise via syringe SOCl₂ (0.5 equiv.) at 0° C. The reaction mixture was allowed to reach room temperature and stirred until consumption of the starting material was observed by TLC analysis, then the reaction was concentrated under reduced pressure and extracted with Et₂O (3×10 mL). The combined organic layers were dried over Na₂SO₄ and the solvent removed in vacuo. The residue was purified by flash column chromatography (silica gel; petroleum ether/EtOAc, 40:60) to obtain the corresponding ester as a colourless liquid (548 mg, 82% yield).

¹H NMR (400 MHz, CDCl₃) δ 7.97-7.94 (d, J=8.3 Hz, 2H), 7.49-7.47 (d, J=8.3 Hz, 2H), 7.43-7.38 (d, J=7.4 Hz, 3H), 7.32-7.29 (t, J=7.4 Hz, 2H), 7.24-7.22 (br, 3H), 7.18-7.16 (br, 3H), 5.55 (s, 1H), 3.89 (s, 3H).

¹³C NMR (101 MHz, CDCl₃) δ 168.82, 146.26, 140.19, 136.39, 130.96, 129.88, 129.07, 128.87, 128.71, 128.49, 128.37, 127.57, 127.00, 57.41, 52.13.

### Preparation of (4-(phenyl(phenylthio)methyl)phenyl)methanol (24)

To a stirred solution of ester 23 (1.0 mmol) in dry THF (10.0 mL) was added dropwise via syringe LiAlH₄ (1.0 mL, 1.0 M solution in Et₂O, 0.5 equiv.) at 0° C. The reaction mixture was allowed to reach room temperature and stirred until consumption of the starting material was observed by TLC analysis, then carefully quenched with saturated aqueous NH₄Cl solution (10 mL) and extracted with Et₂O (3×10 mL). The combined organic layers were dried over Na₂SO₄ and the solvent removed in vacuo. The residue was purified by flash column chromatography (silica gel; petroleum ether/EtOAc, 40:60) to obtain the corresponding alcohol as a brown solid (205 mg, 67% yield), m.p. 178° C. dec.

¹H NMR (400 MHz, CDCl₃) δ 7.43-7.39 (m, 4H), 7.30-7.27 (m, 4H), 7.24-7.22 (m, 3H), 7.19-7.11 (m, 3H), 5.54 (s, 1H), 4.65 (m, 2H), 1.64 (br, 1H).

¹³C NMR (101 MHz, CDCl₃) δ 140.92, 140.52, 139.87, 136.09, 130.43, 128.78, 128.62, 128.60, 128.38, 127.32, 127.25, 126.00, 66.05, 57.11.

### Preparation of ((4-((methylsulfonyl)methyl)phenyl)(phenyl)methyl)(phenyl)sulfane (25)

To a stirred solution of alcohol (144 mg, 0.5 mmol) in dry CH₂Cl₂ (3.0 mL) were added dropwise methanesulphonyl chloride (54 μL, 79 mg, 0.7 mmol, 1.4 equiv.) and triethylamine (97 μL, 70 mg, 0.7 mmol, 1.4 equiv.) at 0° C. The reaction mixture was allowed to reach room temperature and stirred for 12 h, then diluted with H₂O (10 mL) and extracted with CH₂Cl₂ (3×10 mL). The combined organic layers were washed with H₂O, brine and dried over Na₂SO₄. The solvent was removed in vacuo and the crude mixture was purified by flash column chromatography (silica gel; petroleum ether/EtOAc, 90:10) to afford title compound white solid (155 mg, 81% yield), m.p. 144-141° C.

¹H NMR (400 MHz, CDCl₃) δ 7.43-7.39 (m, 3H), 7.31-7.27 (m, 3H), 7.24-7.21 (m, J=7.4 Hz, 3H), 7.18-7.13 (m, 2H), 5.53 (s, 1H), 4.53 (s, 2H), 3.63 (s, 3H).

¹³C NMR (101 MHz, CDCl₃) δ 142.20, 141.32, 139.02, 136.11, 130.57, 128.87, 128.50, 128.31, 128.16, 127.00, 126.77, 126.21, 71.3, 56.2.

### Preparation of ((4-(azidomethyl)phenyl)(phenyl)methyl)(phenyl)sulfane (26)

Mesylate compound 25 (1.5 mmol) was dissolved in DMF (1.0 mL) and sodium azide (1.5 equiv.) was added at 0° C. under N₂ atmosphere. The reaction was allowed to reach room temperature and stirred overnight. Then, H₂O (5 mL) was added, the aqueous layer was extracted with EtOAc (3×10 mL), the combined organic layers were washed with H₂O, brine, dried over Na₂SO₄ and concentrated in vacuo. The residue was purified by flash column chromatography (silica gel; petroleum ether/EtOAc, 70:30) to afford the azide compound as a dense oil (402 mg, 81% yield).

¹H NMR (400 MHz, CDCl₃) δ 7.40-7.38 (m, 4H), 7.33-7.28 (m, 4H), 7.27-7.25 (m, 3H), 7.17-7.11 (m, 3H), 5.53 (s, 1H), 4.30 (s, 2H).

¹³C NMR (101 MHz, CDCl₃) δ 141.97, 141.43, 139.75, 136.13, 130.75, 128.63, 128.37, 128.36, 128.27, 127.41, 126.81, 126.45, 57.10, 54.6.

### Preparation of (4-(phenyl(phenylthio)methyl)phenyl)methanamine (27)

A solution of azide 26 (0.5 mmol) in cyclopentylmethylether (0.25 M) was charged in a round bottomed flask fitted with a magnetic follower and diphenyldisiloxane (DPDS, 1.5 eq.) was then added. To this solution was added PPh₃ (0.1 eq.) and the reaction was left at room temperature for 24 h. Then water was added (10 eq.), the reaction stirred until gas evolution finished and subsequently diluted with Et₂O. 0.5 mL of 4.0 M HCl in dioxane was then added to give compound 27 as the HCl salt (67% yield, 117 mg).

¹H NMR (400 MHz, CDCl₃) δ 7.43-7.32 (m, 4H), 7.31-7.27 (m, 4H), 7.25-7.21 (m, 6H), 5.52 (s, 1H), 4.53 (s, 2H), 2.36 (b, 2.5H).

¹³C NMR (101 MHz, CDCl₃) δ 143.75, 142.66, 138.53, 136.36, 131.67, 128.43, 128.23, 128.11, 128.01, 127.15, 126.95, 126.10, 57.71, 47.81.

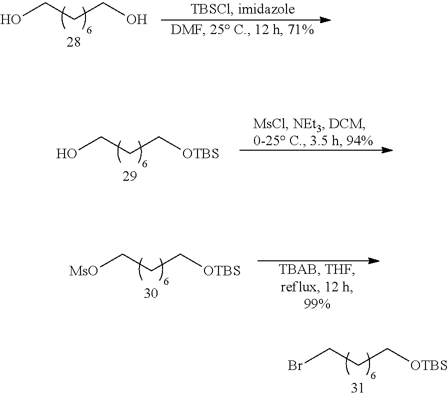

### Preparation of 8-(tert-Butyl-dimethyl-silanyoxy-1-ol) (29)

Imidazole (1.86 g, 27.4 mmol, 2 equiv) and t-butyldimethylsilyl chloride (2.27 g, 15.04 mmol, 1.1 equiv.) were added to a solution of 1,8-octanediol 28 (2.0 g, 13.7 mmol, 1 equiv.) in anhydrous DMF. The reaction mixture was then stirred for 12 h at room temperature. The reaction mixture was poured into ice water and the product was extracted with diethyl ether. The combined diethyl ether extracts were washed with brine, dried over anhydrous Na₂SO₄, filtered and the solvent removed under reduced pressure on a rotary evaporator. Flash column chromatography on silica gel (eluent: 20% EtOAc/pentane, Rf=0.75) yielded purified product 29 (2.5 g, 70%).

¹H NMR (400 MHz, CDCl₃) δ 3.63 (t, J=4.0 Hz, 2H), 1.47-1.59 (m, 4H), 1.31-1.36 (m, 8H), 0.89 (s, 9H), 0.04 (s, 6H). [Matsubara, H.; Maegawa, T.; Kita, Y.; Yokoji, T.; Nomoto, A. Org. Biomol. Chem., 2014, 12, 5442-5447].

### Preparation of 8-((tert-butyldimethylsilyl)oxy)octyl methanesulfonate (30)

Triethylamine (1.6 mL, 11.51 mmol, 2 equiv.) was added to a solution of 29 (1.5 g, 5.76 mmol, 1 equiv) in anhydrous DCM (10 mL) at 0° C., which was then stirred for 30 minutes. Methane sulfonyl chloride (0.67 mL, 18.64 mmol, 1.5 equiv) was added dropwise at 0° C., then the reaction mixture was stirred for 3 hours at room temperature. After completion, the reaction was quenched with water and extracted with DCM. The organic layer was washed with 2 N HCl followed by aq. 10% NaHCO₃ solution and water. The organic layer was dried over anhydrous Na₂SO₄, filtered and the solvent removed under reduced pressure on a rotary evaporator. Flash column chromatography on silica gel yielded purified product 30 (1.84 g, 94%) (eluent: 20% EtOAc/pentane, Rf=0.8).

¹H NMR (400 MHz, CDCl₃) δ 4.22 (t, J=4 Hz, 2H), 3.59 (t, J=8.0 Hz, 2H), 2.99 (s, 3H), 1.71-1.78 (m, 2H), 1.48-1.52 (m, 2H), 1.31-1.41 (m, 8H), 0.89 (s, 9H), 0.04 (s, 6H). [Heathcock, C. H.; Finkelstein, B. L.; Jarvi, E. T.; Radel, P. A; Hardley, C. R. J. Org. Chem. 1988, 53, 1922-42].

### Preparation of ((8-bromooctyl)oxy)(tert-butyl)dimethylsilane (31)

Tetrabutylammonium bromide (2.48 g, 7.7 mmol, 1.5 equiv) was added to a solution of 30 (1.74 g, 5.13 mmol, 1 equiv.) in anhydrous THF (20 mL) at room temperature, which was then refluxed overnight. After completion of reaction, the reaction mixture was allowed to cool to room temperature, then quenched with water and extracted with diethyl ether. The organic layer was dried over anhydrous Na₂SO₄, filtered and the solvent removed under reduced pressure on a rotary evaporator. The resulting crude material was used for the next step without purification. (Starting material was consumed completely (100%); eluent: 10% EtOAc/pentane, Rf=0.8).

¹H NMR (400 MHz, CDCl₃) δ 3.59 (t, J=4.0 Hz, 2H), 3.39 (t, J=8.0 Hz, 2H), 1.81-1.88 (m, 2H), 1.46-1.51 (m, 2H), 1.40-1.43 (m, 2H), 1.30-1.32 (m, 6H), 0.88 (s, 9H), 0.04 (s, 6H). [Jang, C.-S.; Zhou, R.; Gong, J.-X.; Chen, L.-L.; Kurtan, T.; Shen, X.; Guo, Y.-W. Biorg. & Med. Chem. Lett. 2011, 21, 1171-1175.]

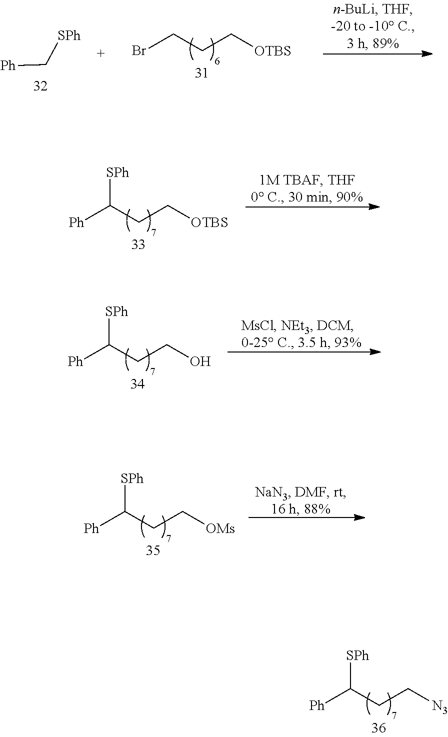

### Preparation of tert-butyldimethyl ((9-phenyl-9-(phenylthio)nonyl)oxy)silane (33)

n-butyl lithium (1.5 equiv., 2.5 M in hexane) was added to a solution of benzyl phenyl thioether (1 equiv.) 32 in anhydrous THF (5 mL) over 5 minutes at −20 to −10° C. under a nitrogen atmosphere. After stirring for 1 hour at the same temperature, alkyl bromide 31 was added to the reaction mixture over 5 minutes at −20 to ˜10° C. The reaction mixture was then stirred for 2 hours at −20 to ˜10° C. After complete consumption of starting material 31 (confirmed by TLC), the reaction was poured into ice water and extracted with diethyl ether. The organic layer was then washed with aq. ammonium chloride solution and concentrated in vacuo. Flash column chromatography on silica gel provided purified products. Purified Yield: 89%, colourless liquid; (eluent: 0.5% EtOAc/pentane, Rf=0.5); ¹H NMR (400 MHz, CDCl₃) δ 6.92-7.46 (m, 10H), 4.11 (dd, J=8.6, 6.3 Hz, 1H), 3.58 (t, J=6.6 Hz, 2H), 1.88-1.99 (m, 2H), 1.43-1.51 (m, 2H), 1.24-1.35 (m, 10H), 0.89 (s, 9H), 0.04 (s, 6H). ¹³C NMR (101 MHz, CDCl₃) δ 142.3, 135.2, 132.2 (2C), 128.5 (2C), 128.2 (2C), 127.7 (2C), 126.9, 126.8, 63.3, 53.6, 36.2, 32.8, 29.4, 29.3, 29.2, 27.5, 25.9 (3C), 25.7, 18.3, −5.3 (2C). HRMS (ESI, ToF) calculated empirical mass (M⁺Na) (C₂₇H₄₂OSSiNa) 465.2623; found 465.2616.

### Preparation of 9-phenyl-9-(phenylthio)nonan-1-ol (34)

To a solution of compound 33 (1 mmol) in THF (5 mL), kept at 0° C. by an ice bath, was added 1 M TBAF solution in THF (1 equiv.) and the reaction mixture was stirred for 30 min. The reaction was then poured into ice water and extracted with diethyl ether. The organic layer was washed with aq. ammonium chloride solution and concentrated in vacuo.

Yield: 90%; white solid, m.p. 181-177° C. ¹H NMR (400 MHz, CDCl₃) δ 7.16-7.28 (m, 10H), 4.11 (dd, J=8.6, 6.3 Hz, 1H), 3.61 (t, J=5.7 Hz, 2H), 1.92-1.96 (m, 2H), 1.50-1.57 (m, 2H), 1.25-1.37 (m, 10H). ¹³C NMR (101 MHz, CDCl₃) δ 142.2, 135.1, 132.2 (2C), 128.5 (2C), 128.2 (2C), 127.7 (2C), 126.9, 126.8, 63.0, 53.6, 36.2, 32.8, 29.3, 29.2, 29.1, 27.5, 25.6. HRMS (ESI, ToF) calculated (M⁺Na) 351.1759; found 351.1754.

### Preparation of 9-phenyl-9-(phenylthio)nonyl methanesulfonate (35)

To the solution of alcohol 34 (1.0 g, 3.0 mmol, 1 equiv.) in anhydrous DCM (10 mL) was added triethylamine (0.84 mL, 6.0 mmol, 2 equiv.) at 0° C., which was then stirred for 30 minutes. Methane sulfonyl chloride (0.35 mL, 4.5 mmol, 1.5 equiv.) was added dropwise at 0° C., then the reaction mixture was stirred for 3 hours at room temperature. After completion the reaction was quenched with water and extracted with DCM. The organic layer was washed with 2 N HCl followed by aq. 10% NaHCO₃ solution and water. The organic layer was dried over anhydrous Na₂SO₄, filtered and the solvent removed under reduced pressure on a rotary evaporator. Flash column chromatography on silica gel provided purified product with yield: 98%; (eluent: 20% EtOAc/pentane, Rf=0.5); white solid, m.p. 165-163; 1H NMR (400 MHz, CDCl₃) δ 7.16-7.27 (m, 10H), 4.19 (t, J=6.6 Hz, 2H), 4.10 (dd, J=8.6, 6.4 Hz, 1H), 2.98 (s, 3H), 1.80-2.02 (m, 2H), 1.62-1.76 (m, 2H), 1.20-1.39 (m, 10H). ¹³C NMR (101 MHz, CDCl₃) δ 142.2, 135.1, 132.3, 128.6, 128.3, 127.7, 126.9, 126.9, 70.1, 53.6, 37.3, 36.2, 29.0, 29.1, 29.1, 28.8, 27.5, 25.3.

### Preparation of (4-azido-2,2,2,2,2-pentamethyl-2-phenyl-2\9-butan-2-yl)(phenyl)sulfane (36)

Mesylate compound 35 (1.5 mmol) was dissolved in DMF (1.0 mL) and then sodium azide (1.5 equiv.) was added at 0° C. under N₂ atmosphere. The reaction was allowed to reach rt and stirred overnight. Then, H₂O (5 mL) was added, the aqueous layer was extracted with EtOAc (3×10 mL), the combined organic layers were washed with H₂O, brine, dried over Na₂SO₄ and concentrated in vacuo. The residue was purified by flash column chromatography (silica gel; petroleum ether/EtOAc, 70:30) to afford 36 as a solid (402 mg, 81% yield), m.p. 121-119° C. ¹H NMR (400 MHz, CDCl₃) δ 7.28-7.15 (m, 10H), 4.18 (t, J=6.6 Hz, 2H), 3.31 (dt, J=12.6, 6.4 Hz, 1H), 2.02-1.81 (m, 2H), 1.76-1.61 (m, 2H), 1.41-1.18 (m, 10H). ¹³C NMR (101 MHz, CDCl₃) δ 142.2, 135.1, 132.3, 128.6, 128.3, 127.7, 126.9, 126.9, 54.6, 49.1, 36.2, 29.0, 29.1, 29.1, 28.8, 27.5, 25.3.

### Preparation of Phenyl(1-phenylhept-6-yn-1-yl)sulfane (38)

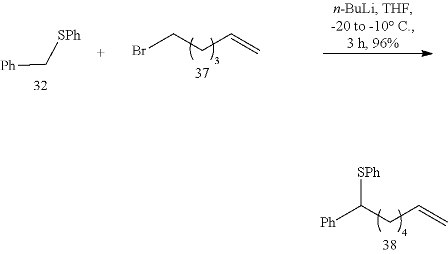

n-butyl lithium (1.5 equiv., 2.5 M in hexane) was added to a solution of benzyl phenyl thioether (1 equiv.) 32 in anhydrous THF (5 mL) over 5 minutes at −20 to ˜10° C. under nitrogen atmosphere. After stirring for 1 hour at the same temperature, alkyl bromide 37 was added to the reaction mixture over 5 minutes at −20 to ˜10° C. The reaction mixture was then stirred for 2 hours at −20 to ˜10° C. After complete consumption of starting material 37 (confirmed by TLC), the reaction was poured into ice water and extracted with diethyl ether. Then, the organic layer was washed with aq. ammonium chloride solution and concentrated in vacuo. Flash column chromatography on silica gel provided purified products. Purified: 100% Pentane, Rf=0.4); colourless liquid; ¹H NMR (400 MHz, CDCl₃) δ 7.38-6.88 (m, 10H), 5.76 (ddt, J=17.0, 10.1, 6.7 Hz, 1H), 5.06-4.86 (m, 2H), 4.12 (dd, J=8.5, 6.5 Hz, 1H), 2.08-1.89 (m, 4H), 1.44-1.26 (m, 4H). ¹³C NMR (101 MHz, CDCl₃) δ 142.2, 138.7, 135.1, 132.3, 128.6, 128.3, 127.7, 126.9, 126.9, 114.39, 53.5, 36.0, 33.5, 28.5, 27.0.

### Preparation of Phenyl(1-phenylhept-6-yn-1-yl)sulfane (40)

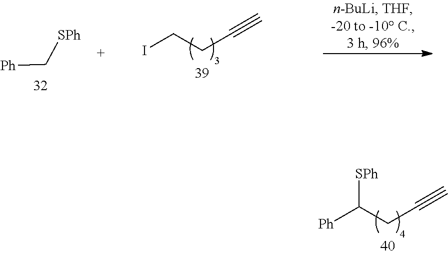

n-butyl lithium (1.5 equiv., 2.5 M in hexane) was added to a solution of benzyl phenyl thioether (1 equiv.) 32 in anhydrous THF (5 mL) over 5 minutes at −20 to ˜10° C. under a nitrogen atmosphere. After stirring for 1 hour at the same temperature, alkyl iodide 39 was added to the reaction mixture over 5 minutes at −20 to ˜10° C. The reaction mixture was then stirred for 2 hours at −20 to ˜10° C. After complete consumption of starting material 37 (confirmed by TLC), the reaction was poured into ice water and extracted with diethyl ether. Then the organic layer was washed with aq. ammonium chloride solution and concentrated in vacuo. Flash column chromatography on silica gel provided purified product with yield: 50%; colourless thick liquid; ¹H NMR (400 MHz, CDCl₃) δ 7.17-7.28 (m, 10H), 4.12 (dd, J=8.6, 6.4 Hz, 1H), 1.91-2.15 (m, 5H), 1.38-1.55 (m, 4H). ¹³C NMR (101 MHz, CDCl₃) δ 142.0, 134.9, 132.4, 128.6, 128.3, 127.7, 127.0, 127.0, 84.2, 68.3, 53.5, 35.7, 28.1, 26.7, 18.2.

### Exemplary Applications of Tags

### 5-(1-(3-phenyl-3-(phenylthio)propyl)-¹H-1,2,3-triazol-4-yl)pyrimidine-2,4(1H,3H)-dione (20)

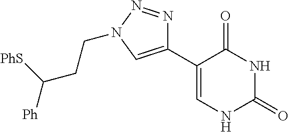

To a solution of (11) (134.7 mg, 0.5 mmol) in degassed DMF (8 mL) were added 5-ethynyluracil (68.1 mg, 0.5 mmol, 1 equiv), copper sulphate pentahydrate (149.8 mg, 0.6 mmol, 1.2 equiv) and sodium ascorbate (297.2 mg, 1.5 mmol, 3 equiv). The mixture was stirred at room temperature for 18 h then concentrated in vacuo (until Vₜₒₜ≈2 mL). The concentrated mixture was diluted with brine (10 mL) then extracted with CH₂Cl₂ (3×20 mL). The combined organic layers were washed with brine (4×10 mL), the resulting organic layer was dried (Na₂SO₄) and concentrated in vacuo. The residue was triturated in Et₂O to afford the desired compound (20) (187.1 mg, 0.46 mmol, 92% yield) as an off-white precipitate.

¹H NMR (400 MHz, DMSO-d6) δ 11.39 (s, 1H), 11.20 (br s, 1H), 8.28 (s, 1H), 8.01 (s, 1H), 7.31-7.19 (m, 10H), 4.45-4.27 (m, 3H), 2.45 (dd, J=14.6, 7.4 Hz, 2H). ³C NMR (101 MHz, DMSO-d6) δ 162.11, 150.60, 140.53, 139.06, 137.57, 134.00, 131.12, 128.96, 128.48, 127.71, 127.44, 127.07, 122.04, 103.86, 48.76, 47.39, 35.88.

### 5-(1-(3-((4-methoxyphenyl)thio)-3-phenylpropyl)-¹H-1,2,3-triazol-4-yl)pyrimidine-2,4(1H,3H)-dione (21)

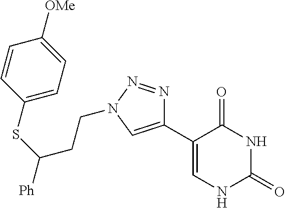 [SMILES: COc1ccc(SC(CCn2cc(-c3c[nH]c(=O)[nH]c3=O)nn2)c2ccccc2)cc1]

To a solution of (12) (598.8 mg, 2.0 mmol) in degassed DMF (30 mL) were added 5-ethynyluracil (272.2 mg, 2.0 mmol, 1 equiv), copper sulphate pentahydrate (599.3 mg, 2.4 mmol, 1.2 equiv) and sodium ascorbate (1.19 g, 6.0 mmol, 3 equiv). The mixture was stirred at room temperature for 18 h then concentrated in vacuo (until Vₜₒₜ≈5 mL). The concentrated mixture was diluted with brine (30 mL) then extracted with CH₂Cl₂ (3×75 mL). The combined organic layers were washed with brine (4×20 mL), the resulting organic layer was dried (Na₂SO₄) and concentrated in vacuo. The residue was triturated in Et₂O to afford the desired compound (21) (365.9 mg, 0.84 mmol, 42% yield) as a pale green precipitate.

¹H NMR (400 MHz, DMSO-d6) δ 11.39 (s, 1H), 11.19 (br s, 1H), 8.26 (s, 1H), 8.00 (s, 1H), 7.31-7.21 (m, 5H), 7.16 (d, J=8.7 Hz, 2H), 6.82 (d, J=8.7 Hz, 2H), 4.37 (t, J=7.0 Hz, 2H), 4.09 (t, J=7.5 Hz, 1H), 3.71 (s, 3H), 2.41 (virt dquint, J=14.0, 7.1 Hz, 2H). ¹³C NMR (101 MHz, DMSO δ 162.10, 159.23, 150.59, 140.71, 139.02, 137.51, 135.11, 128.38, 127.70, 127.30, 123.52, 122.01, 114.52, 103.88, 55.13, 50.33, 47.43, 35.46.

### 2R,3R,4R,5R)-2-((benzoyloxy)methyl)-5-(2,4-dioxo-5-(1-(3-phenyl-3-(phenylthio)propyl)-1H-1,2,3-triazol-4-yl)-3,4-dihydropyrimidin-1 (2H)-yl)tetrahydrofuran-3,4-diyl dibenzoate (22)

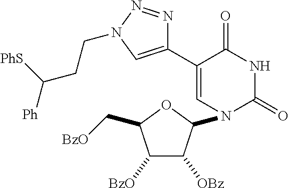

To a solution of (11) (150.4 mg, 0.56 mmol) in degassed DMF (15 mL) were added 2′,3′,5′-tribenzoate-5-ethynyluridine (324.2 mg, 0.56 mmol, 1 equiv), copper sulphate pentahydrate (167.3 mg, 0.67 mmol, 1.2 equiv) and sodium ascorbate (331.9 mg, 1.68 mmol, 3 equiv). The mixture was stirred at room temperature for 18 h then concentrated in vacuo (until Vₜₒₜ≈5 mL). The concentrated mixture was diluted with brine (15 mL) then extracted with CH₂Cl₂ (3×40 mL). The combined organic layers were washed with brine (4×10 mL), the resulting organic layer was dried (Na₂SO₄) and concentrated in vacuo. The residue was purified by flash chromatography on silica gel (Petroleum Ether/AcOEt-6:4) to afford the desired compound (22) (273.5 mg, 0.32 mmol, 58% yield) as a white foamy solid.

¹H NMR (400 MHz, CDCl₃) 8.91 (b, 1H), 8.51 (s, 1H), 8.28-8.18 (m, 3H), 7.95 (d, J=8, 2H), 7.84 (d, J=8, 2H), 7.65-7.21 (m, 18H), 6.21 (s, 1H), 5.86 (s, 2H), 4.81-4.78 (m, 3H), 4.48-4.32 (m, 2H), 4.14 (t, J=7, 1H), 2.26-2.49 (m, 2H); ¹³C NMR (400 MHz, CDCl₃) 166.4, 165.5, 165.3, 161.0, 130.1, 130.0, 130.0, 128.9, 128.8, 128.6, 128.6, 128.0, 127.9, 122.7, 107.2, 90.8, 88.6, 74.0, 71.2, 63.9, 50.5, 48.2, 36.4.

### (2R,3R,4R,5R)-2-((benzoyloxy)methyl)-5-(5-(1-(3-((4-methoxyphenyl)thio)-3-phenylpropyl)-1H-1,2,3-triazol-4-yl)-2,4-dioxo-3,4-dihydropyrimidin-1 (2H)-yl)tetrahydrofuran-3,4-diyl dibenzoate (23)

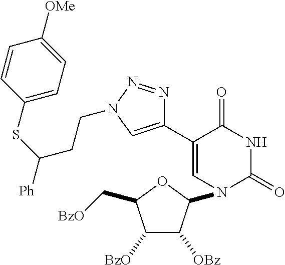

To a solution of (12) (407.6 mg, 1.36 mmol) in degassed DMF (25 mL) were added 2′,3′,5′-tribenzoate-5-ethynyluridine (790.4 mg, 1.36 mmol, 1 equiv), copper sulphate pentahydrate (407.9 mg, 1.63 mmol, 1.2 equiv) and sodium ascorbate (809.1 mg, 4.08 mmol, 3 equiv). The mixture was stirred at room temperature for 18 h then concentrated in vacuo (until Vₜₒₜ≈5 mL). The concentrated mixture was diluted with brine (25 mL) then extracted with CH₂Cl₂ (3×60 mL). The combined organic layers were washed with brine (4×15 mL), the resulting organic layer was dried (Na₂SO₄) and concentrated in vacuo. The residue was purified by flash chromatography on silica gel (Petroleum Ether/AcOEt-6:4) to afford the desired compound (23) (924.2 mg, 1.05 mmol, 77% yield) as a white foamy solid.

¹H NMR (400 MHz, CDCl₃) 8.49 (s, 1H), 8.81 (s, 1H), 8.28 (d, J=8, 2H), 8.20 (s, 1H), 8.01 (d, J=8, 2H), 7.86 (d, J=7, 2H), 7.66-7.21 (m, 11H), 7.18 (d, J=7, 4H), 6.76 (d, J=7, 2H), 6.28 (s, 1H), 5.94 (m, 2H), 5.80-5.74 (m, 3H), 4.49-4.30 (m, 2H), 3.85-3.80 (m, 1H), 3.73 (s, 3H), 2.26-2.24 (m, 2H); ¹³C NMR (400 MHz, CDCl₃) 166.3, 165.3, 165.2, 164.3, 161.2, 160.5, 160.0, 149.0, 136.3, 130.0, 129.9, 129.8, 128.6, 128.5, 128.5, 128.8, 127.6, 122.51, 114.4, 90.6, 90.1, 73.9, 71.1, 63.7, 55.3, 51.4, 48.2 35.9.

### (2R,3R,4R,5R)-2-((benzoyloxy)methyl)-5-(5-(1-(3-fluoro-3-phenylpropyl)-¹H-1,2,3-triazol-4-yl)-2,4-dioxo-3,4-dihydropyrimidin-1 (2H)-yl)tetrahydrofuran-3,4-diyl dibenzoate (24)

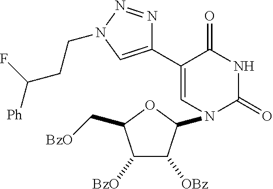

In a plastic vessel containing a suspension of N-bromosuccinimide (15.8 mg, 0.09 mmol, 1.2 equiv.) in CH₂Cl₂ (0.5 mL) at −20° C. under N₂ was added HF/pyridine complex (ca. 70% HF) (75 μL). After 5 min, a solution of 22 (62.7 mg, 0.074 mmol, 1 equiv.) or 23 (65.1 mg, 0.074 mmol, 1 equiv.) in CH₂Cl₂ (0.5 mL) was added dropwise. After 20 min of stirring at 20° C., the reaction was quenched by addition of a saturated solution of sodium thiosulfate (5 mL) and the mixture was extracted thrice with CH₂Cl₂ (3×15 mL). The combined organic layers were dried over Na₂SO₄ and concentrated in vacuo. The residue was purified by flash chromatography on silica gel (Petroleum Ether/AcOEt-6:4) to afford the desired compound (42.8 mg, 74% from 21/46.0 mg, 0.041 mmol, 80% from 23) as a white foamy solid.

¹H NMR (400 MHz, CDCl₃) 9.10 (s, 1H), 8.51 (s, 1H), 8.25 (s, 1H), 8.31 (d, J=7, 2H), 7.99 (d, J=7, 2H), 7.82 (d, J=7, 2H), 7.59-7.49 (m, 3H), 7.48-7.29 (m, 10H), 6.26 (s, 1H), 5.92 (s, 2H), 5.40 (dm, J=48, 1H), 4.83-4.76 (m, 3H), 4.59-4.51 (m, 2H), 2.59-2.45 (2H). ¹³C NMR (400 MHz, CDCl₃) 177.5, 166.4, 165.5, 165.3, 161.0, 149.3, 133.9, 133.8, 133.4, 130.0, 129.9, 128.9, 128.8, 128.7, 128.6, 128.6, 128.5, 125.5, 107.1, 92.1, 90.1, 90.4, 80.6, 74.0, 63.9, 46.5, 37.8, 37.6, 29.7, 14.3. ¹⁹F NMR (376 MHz, CDCl₃): 0-178.7.

### Exemplary Preparations of Fluorinable Amino Acids.

Starting either from(S)-41 or (R)-41, the synthesis of which has been reported [Corey, E. J., Xu, F.; Noe, M. C. J. Am. Chem. Soc. 1997, 119, 12414-12415]. Synthesis herein exemplified for the(S) enantiomer.

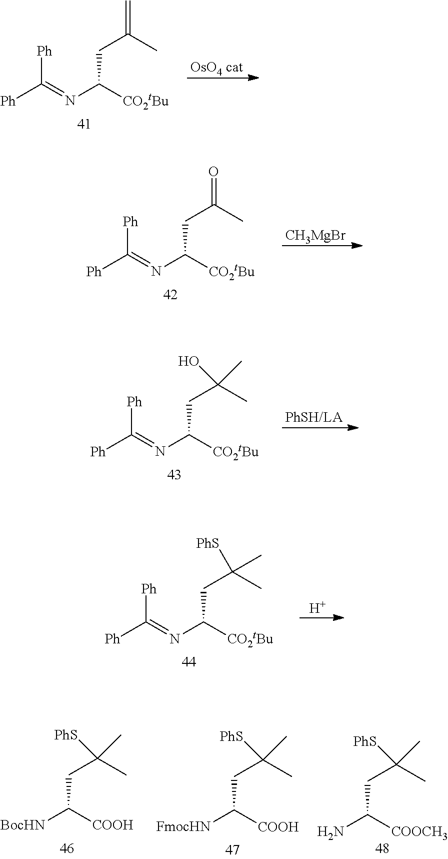

### Preparation of tert-butyl (S)-2-((diphenylmethylene)amino)-4-oxopentanoate 42

To a solution of compound 42 (1.5 g, 4.29 mmol) in dioxane-water (3:1, 30 mL) were added 2,6-lutidine (1 mL, 8.58 mmol), OsO₄ (2.5% in t-BuOH, 872 μL, 0.0858 mmol) and NalO₄ in a portion-wise manner (3.67 g, 17.16 mmol). The reaction was stirred at room temperature and monitored by TLC. After 4 h the reaction mixture was quenched with sodium sulfite (saturated solution), then CH₂Cl₂ and water were added to the reaction mixture. The organic layer was washed with water (×3) and brine (×2). Then, the organic layer was dried over Na₂SO₄, filtered and concentrated under vacuum. The crude product was analysed by NMR. The colourless solid was obtained after purification on silica gel column chromatography (EtOAc: petroleum ether 10:90), m.p. 124-5° C. (1.419 g, 94% yield). ¹H NMR (400 MHz, CDCl₃) δ 7.62-7.57 (m, 2H), 7.52-7.22 (m, 6H), 6.95 (d, J=7.7 Hz, 2H), 4.45 (dd, J=7.1, 5.9 Hz, 1H), 3.14 (dd, J=17.0, 5.8 Hz, 1H), 2.94 (dd, J=17.0, 7.2 Hz, 1H), 2.16 (s, 3H), 1.43 (s, 9H). ¹³C NMR (101 MHz, CDCl₃) 202.7, 173.8, 166.2, 136.5, 134.8, 130.9, 129.2, 129.1, 128.7, 128.6, 128.0, 84.5, 69.5, 41.9, 29.1, 27.8.

### Preparation of tert-butyl(S)-2-((diphenylmethylene)amino)-4-hydroxy-4-methylpentanoate 43

A 3.0 M solution of methyl magnesium bromide in Et₂O (1 mL, 1 equiv.) was added to a solution of compound 42 (1.053 g, 3 mmol) in dry THF (10 mL), kept at −78° C. by an acetone dry ice bath, during a period of 5 min. The reaction was stirred at −78° C. for one hour then allowed to react at room temperature, stirred for an additional hour, then quenched with saturated aqueous NH₄Cl. The crude mixture was extracted with EtOAc (3×5 mL), the organic layer dried over Na₂SO₄, and the oil so obtained purified by flash chromatography (eluent AcOEt: petroleum ether 25:75) to give 43 as a dense oil (792 mg, 72% yield). ¹H NMR (400 MHz, CDCl₃) δ 7.61-7.20 (m, 8H), 6.93 (d, J=7.4 Hz, 2H), 4.43 (dd, J=7.0, 5.0 Hz, 1H), 1.75 (m, 2H) 1.43 (s, 9H), 1.25 (s, 6H); ¹³C NMR (101 MHz, CDCl₃) 175.1, 165.8, 134.6, 134.1, 131.0, 129.8, 129.0, 128.9, 128.8, 128.0, 84.2, 69.5, 46.9, 29.8, 28.1.

### Preparation of tert-butyl(S)-2-((diphenylmethylene)amino)-4-methyl-4-(phenylthio) pentanoate 44

Thiophenol (1.2 eq.) and BF₃Et₂O (2.0 eq.) were added to a stirred solution of alcohol 43 (2.0 mmol) in dry THF (4.0 mL), and the reaction mixture was stirred at room temperature until complete consumption of the starting material was observed by TLC analysis (24 h). The reaction mixture was evaporated and the residue purified by flash column chromatography on silica gel (EtOAc: Petroleum Ether 10:90) to give compound 44 as a colourless oil (789 mg, 86% yield). ¹H NMR (400 MHz, CDCl₃) δ 7.66-7.23 (m, 8H), 7.21-6.80 (m, 7H), 4.02 (m, 1H), 1.68 (m, 2H), 1.42 (s, 9H), 1.38 (s, 6H); ¹³C NMR (101 MHz, CDCl₃) 174.8, 166.1, 135.1, 134.8, 134.1, 131.8, 131.6, 131.2, 129.8, 129.7, 129.1, 128.7, 128.1, 127.9, 84.2, 66.1, 44.1, 33.1, 29.1, 28.4.

### Preparation of(S)-2-amino-4-methyl-4-(phenylthio)pentanoic acid 45

Aqueous HCl (3 M, 30 mL) was added to a solution of 44 (700 mg) in ether (10 mL) at room temperature under N₂ and the biphasic mixture was stirred overnight (18 h). The organic layer was separated, washed with CH₂Cl₂ (2×10 mL) and the aqueous layer was lyophilized to give compound 45 as a solid; m.p. 220-218° C.; ¹H NMR (400 MHz, CDCl₃)· 12.23 (br, 1H), 7.41-7.26 (m, 5H), 5.76 (br, 2H), 3.23 (dd, J=6, J=4, 1H), 2.12 (dd, J=12, J=6), 1.98 (dd, J=12, J=4), 1.31 (s, 6H); ¹³C NMR (101 MHz, CDCl₃) 175.7, 136.2, 131.4, 128.8, 128.5, 45.8, 36.1, 33.8, 29.4.

### Preparation of(S)-2-((tert-butoxycarbonyl)amino)-4-methyl-4-(phenylthio)pentanoic acid 46

A solution of 45 (100 mg), in methanol (3 mL) was treated with triethylamine (1 equiv.) and with di-tert-butyl-dicarbonate (1.2 equiv.). The reaction mixture was allowed to react at room temperature for 2 hours, then the solvent was evaporated, the crude washed with saturated NH₄Cl, the organic layer dried over Na₂SO₄ and evaporated to give pure 46 as a wax (99% yield). ¹H NMR (400 MHz, CDCl₃) δ (12.10 br, 1H), 7.39-7.21 (m, 5H), 4.12 (dd, J=7, J=3, 1H), 2.12 (dd, J=10, J=7), 1.98 (dd, J=10, J=3), 1.45 (s, 9H) 1.34 (s, 6H); ¹³C NMR (101 MHz, CDCl₃) 176.6, 156.9, 135.7, 130.9, 128.6, 128.3, 78.9, 51.7, 41.2, 37.7, 29.3, 28.1.

### Preparation of (S)-2-((((9H-fluoren-9-yl) methoxy) carbonyl)amino)-4-methyl-4-(phenylthio)pentanoic acid 47

A solution of 45 (100 mg), in methanol (3 mL) was treated with triethylamine (1 equiv) and with FmocCl (1.2 equiv.). The reaction mixture was allowed to react at room temperature for 1 hour, then the solvent was evaporated, the crude washed with water, the organic layer dried over Na₂SO₄ and evaporated to give pure 47 as a wax (98% yield). ¹H NMR (400 MHz, CDCl₃) δ 12.0 (br, 1H), 7.55-7.23 (m, 13H), 4.73 (m, 2H), 4.37 (m, 1H), 4.34 (dd, J=7, J=5, 1H), 2.08 (dd, J=8, J=7), 1.92 (dd, J=8, J=5), 1.36 (s, 6H); ¹³C NMR (101 MHz, CDCl₃) 175.1, 171.6, 144.3, 142.9, 135.1, 131.7, 128.2, 128.0, 126.1, 125.4, 124.8, 120.0, 53.1, 43.3, 41.2, 37.6, 28.3, 21.0.

### Preparation of methyl (S)-2-amino-4-methyl-4-(phenylthio)pentanoate 48

A solution of 45 (100 mg), in methanol (3 mL) was treated with acetyl chloride (0.1 equiv.) and the resulting solution was stirred overnight at room temperature. The reaction was then evaporated, the crude was taken up in ethyl acetate (10 mL), washed with saturated NaHCO₃ aqueous solution and the organic layer evaporated to give compound 48 as an oil (99% yield). ¹H NMR (400 MHz, CDCl₃) δ 8.24 (br, 2H), 7.45-7.23 (m, 5H), 3.71 (s, 3H), 3.75 (m, 1H), 2.15-2.10 (m, 2H), 1.33 (m, 6H); ¹³C NMR (101 MHz, CDCl₃) 177.1, 135.8, 131.2, 128.7, 128.1, 51.2, 49.1, 36.8, 34.1, 29.9.

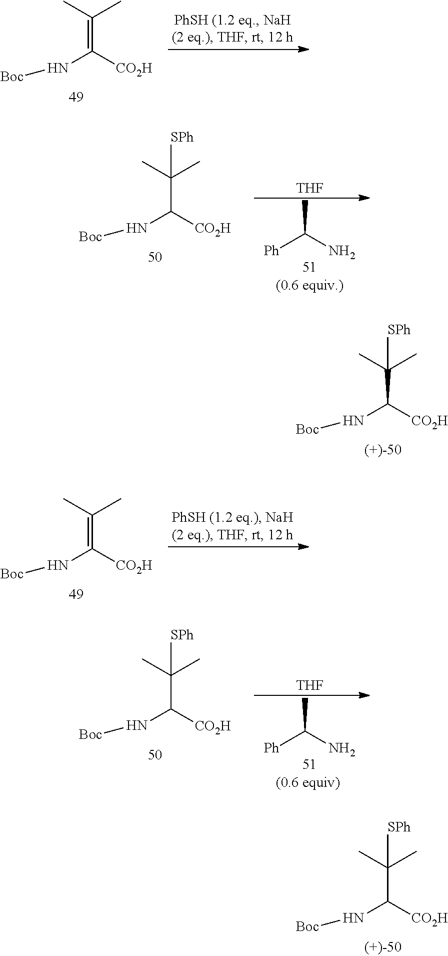

### Preparation of 2-((tert-butoxycarbonyl)amino)-3-methyl-3-(phenylthio) butanoic acid 50

The preparation of compound 49 has been described [Poisel, H, Chem. Berichte, 1977, 110, 948-953]. NaH (2.0 equiv) and PhSH (1.2 equiv.) were sequentially added to a solution of compound 49 (645 mg, 3 mmol) in 10 mL of THF, kept at 0° C. by an ice bath. The reaction mixture was then allowed to reach room temperature and stirred for 12 hours. Then, the solvent was evaporated, the residue was taken up in ethyl acetate (5 mL), washed with sat NH₄Cl solution, the organic layer collected, dried over Na₂SO₄ and evaporated to give pure compound 50 as a wax (887 mg, 91% yield). ¹H NMR (400 MHz, CDCl₃) δ 12.2 (br, 1H), 7.28-7.23 (m, 5H), 5.21 (br, 1H), 4.45 (s, 1H), 1.62 (s, 6H), 1.45 (s, 9H); ¹³C NMR (101 MHz, CDCl₃) 175.1, 155.5, 135.1, 131.4, 128.2, 128.6, 79.1, 71.1, 45.2, 28.9, 22.8.

### Resolution of compound 50, isolation of (+)-2-((tert-butoxycarbonyl)amino)-3-methyl-3-(phenylthio) butanoic acid (+)-50

Racemate 50 (650 mg, 2 mmol) was dissolved in THF (10 mL) and (R)-1-phenylethan-1-amine (0.6 equiv, 145 mg) added. The solution so obtained was refluxed for 5 min. The solution was then allowed to reach room temperature. After 6 h a solid precipitated and was collected, washed with dry Et₂O and confirmed by ¹H-NMR to be a single diasteroisomer (225 mg, 26% yield). This salt was suspended in DCM (5 mL), treated with sat. aq. NH₄Cl, the organic layer separated, dried over Na₂SO₄ and evaporated to give compound (+)−50; [α]_{D}²⁰=+54.

### Dimethyl (2S,4S)-2-((tert-butoxycarbonyl)amino)-4-(phenylthio) pentanedioate

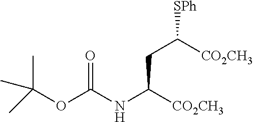

Thiophenol (1.2 eq.) and BF₃Et₂O (2.0 eq.) were added to a stirred solution of Dimethyl (2S)-2-((tert-butoxycarbonyl)amino)-4-hydroxypentanedioate (600 mg, 2.06 mmol) in dry THF (4.0 mL), and the reaction mixture was stirred at room temperature until complete consumption of the starting material was observed by TLC analysis (24 h). The reaction mixture was evaporated and the residue purified by flash column chromatography on silica gel (EtOAc: Petroleum Ether 10:90) to give title compound as a colourless oil (639 mg, 81% yield).

¹H NMR (400 MHz, CDCl₃) 7.49-7.45 (2H, m), 7.32-7-26 (m, 3H), 4.96 (app d, J=8 Hz, 1H), 4.63 (app t, J=4 Hz, 1H), 3.72 (s, 3H), 3.68 (s, 3H), 2.30-2.23 (m, 1H), 2.14 (ddd, J=16, 8, 6 Hz), 1.43 (s, 9H).

### Numbered Clauses

Further embodiments of the present invention are described in the following numbered clauses.

1. A process for incorporating a fluorine atom into a molecule, said process comprising converting a compound of Formula (I) into a compound of Formula (II):

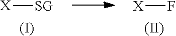

wherein G is an optionally substituted C₁-C₆ alkyl group, an optionally substituted aryl group, or an optionally substituted heteroaryl group, and X is an organic group; and wherein the SG group is attached to a secondary or tertiary carbon atom in the organic group X; said process comprising treating said compound of Formula (I) with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium; and
(ii) a source of fluoride.

2. The process according to clause 1 which comprises simultaneously treating the compound of Formula (I) with said activator and said source of fluoride.

3. The process according to any preceding clause, wherein the fluorine is ¹⁸F.

4. The process according to any preceding clause, wherein G is a phenyl or heteroaryl group optionally substituted by one or more substituents independently selected from alkoxy, nitro, halo, and alkyl, or wherein G is selected from methyl and trifluoromethyl.

5. The process according to any preceding clause, wherein G is selected from phenyl, ortho-alkoxyphenyl, para-alkoxyphenyl, and ortho, para-dialkoxyphenyl; preferably wherein G is selected from phenyl and para-methoxyphenyl.

6. The process according to any preceding clause, wherein the activator compound is selected from the group consisting of N-bromosuccinimide, N-chlorosuccinimide, N-fluorobenzenesulfonimide, N-chlorobenzenesulfonimide, N-bromobenzenesulfonimide, diethylaminosulfur trifluoride, diethylaminodifluorosulfinium tetrafluoroborate, morpholinodifluorosulfinium tetrafluoroborate, and bromine; and is more preferably, N-bromosuccinimide or N-chlorosuccinimide.

7. The process according to any preceding clause, wherein the source of fluoride is a fluoride phase transfer catalyst.

8. The process according to any one of clauses 1 to 6, wherein the source of fluoride is selected from the group consisting of metal fluoride salts, complexes or chelates of hydrogen fluoride or metal fluoride salts, and tetraalkylammonium fluorides.

9. The process according to any one of clauses 1 to 6, wherein the source of fluoride is a metal fluoride salt, preferably an alkali metal salt.

10. The process according to any one of clauses 1 to 6, wherein the source of fluoride is pyridine-HF.

11. The process according to any one of clauses 1 to 6, wherein the source of fluoride is a crown ether complex of a metal fluoride, preferably a crown ether complex of an alkali metal fluoride.

12. The process according to any one of clauses 1 to 6, wherein the source of fluoride is a C₁-C₄ tetraalkylammonium fluoride, preferably wherein the source of fluoride is tetrabutylammonium fluoride.

13. The process according to any one of clauses 1 to 6, wherein the source of fluoride is pyridine-HF and the activator is N-bromosuccinimide.

14. The process according to any one of clauses 1 to 6, wherein the activator and the source of fluoride are the same.

15. The process according to clause 14, wherein the activator and the source of fluoride are N-fluorobenzenesulfonimide.

16. The process according to any preceding clause, wherein the process is carried out in an organic solvent, an aqueous solvent, or combinations thereof; preferably wherein the process is carried out in dichloromethane, tetrahydrofuran, dimethylsulfoxide, acetonitrile, benzonitrile, ethanol, methanol, toluene, water, and combinations thereof.

17. The process according to any preceding clause, wherein the process is carried out at room temperature.

18. The process according to any preceding clause wherein the organic group X is of Formula L-Y, where L is a direct bond or a covalent linker group, and Y is selected from the group consisting of a labelling agent, a dye, an amino acid, a peptide, a peptoid, a drug molecule or fragment thereof, an antibody or fragment thereof, a protein, a carbohydrate, a lipid, a nucleobase, a nucleoside, a nucleotide, an oligonucleotide, a polynucleotide, a peptide nucleic acid, and derivatives thereof.

19. The process according to any preceding clause, which process comprises the step of converting a compound of Formula (IIa) to a compound of Formula (Ia):

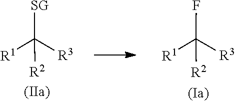

wherein:
R¹ and R² are each independently an optionally substituted hydrocarbyl group;
R³ is H or an optionally substituted hydrocarbyl group;
wherein any two of R¹, R², and R³ or all of R¹, R², and R³ may optionally form one or more cyclic groups.

20. The process according to clause 19 wherein:
R¹ and R² are each independently an optionally substituted aliphatic group; and
R³ is H or an optionally substituted aliphatic group.

21. The process according to clause 19 wherein:
R¹ is an optionally substituted aryl or heteroaryl group;
R² is an optionally substituted aliphatic group; and
R³ is H or an optionally substituted aliphatic group.

22. The process according to clause 19, wherein the carbon to which the R¹, R², R³ and SG groups are bonded in Formula (IIa) is a stereogenic centre and the conversion of the compound of Formula (IIa) to Formula (Ia) results in at least partial inversion of the stereochemical configuration of the corresponding carbon atom to which F is bonded in Formula (Ia) with respect to that of Formula (IIa); preferably complete inversion.

23. The process according to any one of clauses 1 to 22 for incorporating a fluorine atom into a peptide, said process comprising the steps of:
(a) incorporating at least one compound of Formula (III) into the backbone of a peptide to form a peptide precursor,

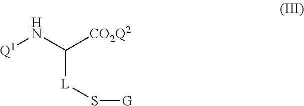

wherein:
Q¹ is H or an amine protecting group;
Q² is H or a carboxyl protecting group;
L is a linker group; and
G is an optionally substituted C₁-C₆ alkyl group, an optionally substituted aryl group, or an optionally substituted heteroaryl group; and
(b) treating the peptide precursor formed in step (a) with:
   (i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium; and
   (i) a source of fluoride.

24. The process according to any one of clauses 1 to 22 for incorporating a fluorine into a peptoid, said process comprising the steps of:
(a) incorporating at least one compound of Formula (IV) into the backbone of a peptoid to form a peptoid precursor,

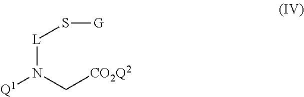

wherein:
Q¹ is H or an amine protecting group;
Q² is H or a carboxyl protecting group;
L is a linker group; and
G is an optionally substituted C₁-C₆ alkyl group, an optionally substituted aryl group, or an optionally substituted heteroaryl group; and
(b) treating the peptoid precursor formed in step (a) with:
   (i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium; and
   (i) a source of fluoride.

25. The process according to clause 23 or clause 24 wherein L is a linker group (CR⁴R⁵) n where n is an integer from 1 to 10, and each R⁴ and R⁵ is independently selected from H, alkyl, aryl and COOH, NH₂.

26. The process according to clause 23 or clause 25 wherein said compound of Formula (III) is selected from:

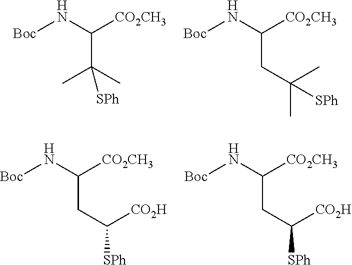

27. The process according to any one of clauses 23, 25 or 26, wherein step (a) comprises preparing the peptide precursor by solid phase peptide synthesis.

28. The process according to any one of clauses 1 to 22 for incorporating a fluorine atom into an oligo- or poly-nucleotide, the process comprising:
(a) preparing an oligo- or poly-nucleotide precursor comprising at least one —SG group, wherein G is an optionally substituted C₁-C₆ alkyl group, an optionally substituted aryl group, or an optionally substituted heteroaryl group;
(b) treating the oligo- or poly-nucleotide precursor formed in step (a) with:
   (i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium; and
   (ii) a source of fluoride.

29. The process according to clause 28 wherein the —SG group is attached via a covalent linker group L to the nucleobase of at least one nucleotide in the oligo- or poly-nucleotide.

30. The process according to clause 28 or clause 29 wherein the —SG group is incorporated by reacting a compound of Formula (V),

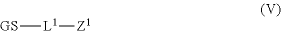

wherein:
Z¹ is a functional group selected from NH₂, N₃, COOH and OH;
L¹ is a linker group (CR⁴R⁵), where n is an integer from 1 to 10;
each R⁴ and R⁵ is independently selected from H, alkyl and aryl; and
G is an optionally substituted C₁-C₆ alkyl group, an optionally substituted aryl group, or an optionally substituted heteroaryl group;
with a reactive group on the nucleobase.

31. The process according to clause 30 wherein the —SG group is incorporated by reacting a compound of Formula (Va),

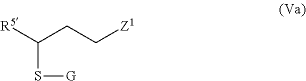

wherein:
Z¹ is a functional group selected from NH₂, N₃, COOH and OH; and
R^{5′} is an aryl group or a tertiary alkyl group;
with a reactive group on the nucleobase.

32. The process according to clause 30 or clause 31 wherein Z¹ is N₃ and the reactive group on the nucleobase is a-C≡CH group.

33. The process according to clause 30 or clause 31 wherein the compound of Formula (V) is selected from the following:

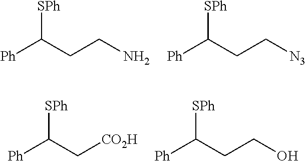

34. The process according to any one of clauses 1 to 22 for incorporating a fluorine atom into a molecule, the process comprising:
(a) preparing a precursor molecule comprising at least one —SG group, wherein G is an optionally substituted C₁-C₆ alkyl group, an optionally substituted aryl group, or an optionally substituted heteroaryl group;
(b) treating the precursor molecule formed in step (a) with:
   (i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium; and
   (ii) a source of fluoride.

35. A process according to clause 34 wherein the —SG group is incorporated into the precursor molecule by reacting a compound of Formula (VI),

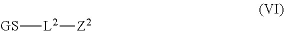

wherein:
Z² is a functional group selected from NH₂, NHR⁶, N₃, COOH, COOR⁷, OH, CN, CONHR⁸, COR⁹, CHO, CSNHR¹⁰;
L² is a linker group (CR⁴R⁵), where n is an integer from 1 to 10;
each R⁴ and R⁵ is independently selected from H, alkyl and aryl;
R⁶, R⁷, R⁸, R⁹, and R¹⁰ are each independently selected from alkyl, aryl and aralkyl; and
G is an optionally substituted C₁-C₆ alkyl group, an optionally substituted aryl group, or an optionally substituted heteroaryl group;
with a reactive group in the molecule.

36. The process according to clause 35 wherein the —SG group is incorporated into the precursor molecule by reacting a compound of Formula (VIa),

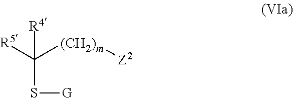

wherein m is 1, 2 or 3, and
wherein R^{4′} is an aryl group or a tertiary alkyl group and R^{5′} is H, an aryl group or a tertiary alkyl group; or wherein R^{4′} and R^{5′} are both alkyl groups;
   with a reactive group in the molecule.

37. A process according to clause 36 wherein the compound of Formula (VIa) is selected from the following:

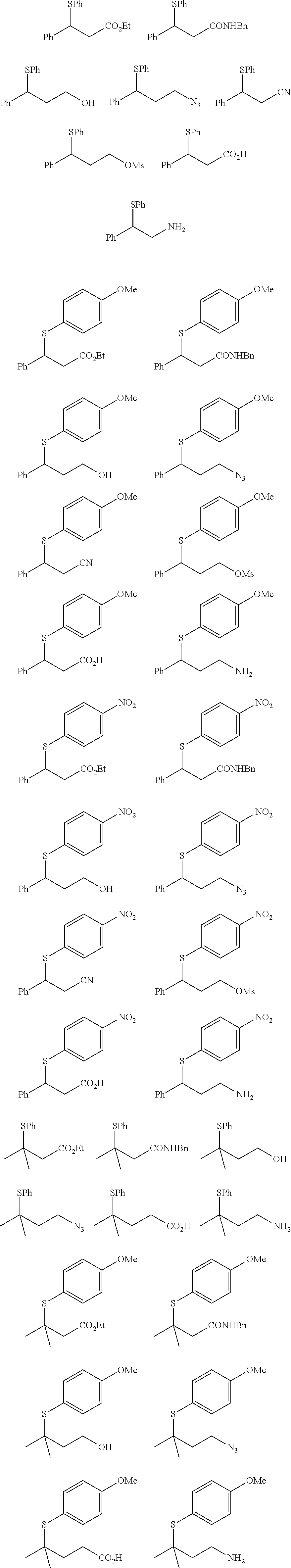

38. The process according to any one of clauses 34 to 37 wherein the molecule is selected from the group consisting of nucleosides, nucleotides, oligo- or poly-nucleotides, peptide nucleic acids, amino acids, mono-oligo- and poly-saccharides, peptides, peptoids, proteins and small molecules.

39. Use of a process according to any one of clauses 1 to 22 in the preparation of a fluorinated amino acid or a fluorinated peptide.

40. Use of a process according to any one of clauses 1 to 22 in the preparation of a fluorinated oligonucleotide or polynucleotide.

41. Use of a process according to any one of clauses 1 to 22 in the preparation of a fluorinated pharmacologically active agent, or a fragment thereof, or a precursor or intermediate thereof.

42. Use of a process according to any one of clauses 1 to 22 in the preparation of a fluorinated radiolabelling agent.

43. Use of a compound of Formula (III)

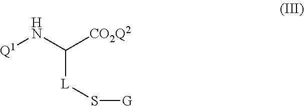

wherein:
Q¹ is H or an amine protecting group;
Q² is H or a carboxyl protecting group;
L is a linker group (CR⁴R⁵), where n is an integer from 1 to 10;
each R⁴ and R⁵ is independently selected from H, alkyl, aryl, COOH, and NH₂; and
G is an optionally substituted C₁-C₆ alkyl group, an optionally substituted aryl group, or an optionally substituted heteroaryl group;
as an intermediate in a process is for preparing fluorinated peptides, wherein said process comprises a step of converting a carbon-SG bond to a carbon-F bond by treating with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium; and
(ii) a source of fluoride.

44. Use of a compound of Formula (IV)

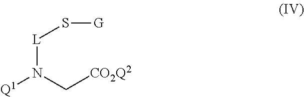

wherein:
Q¹ is H or an amine protecting group;
Q² is H or a carboxyl protecting group;
L is a linker group; and
G is an optionally substituted C₁-C₆ alkyl group, an optionally substituted aryl group, or an optionally substituted heteroaryl group;
as an intermediate in a process is for preparing fluorinated peptoids, wherein said process comprises a step of converting a carbon-SG bond to a carbon-F bond by treating with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium; and
(ii) a source of fluoride.

45. Use of a compound of Formula (V),

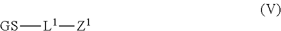

wherein:
Z¹ is a functional group selected from NH₂, N₃, COOH and OH;
L¹ is a linker group (CR⁴R⁵), where n is an integer from 1 to 10;
each R⁴ and R⁵ is independently selected from H, alkyl and aryl; and
G is an optionally substituted C₁-C₆ alkyl group, an optionally substituted aryl group, or an optionally substituted heteroaryl group;
as an intermediate in a process is for preparing a fluorinated oligo- or poly-nucleotide, wherein said process comprises a step of converting a carbon-SG bond to a carbon-F bond by treating with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium; and
(ii) a source of fluoride.

46. Use of a compound of Formula (VI),

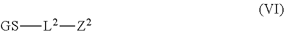

wherein:
Z² is a functional group selected from NH₂, NHR⁶, N₃, COOH, COOR⁷, OH, CN and CONHR⁸, COR⁹, CHO, and CSNHR¹⁰;
L² is a linker group (CR⁴R⁵), where n is an integer from 1 to 10;
each R⁴ and R⁵ is independently selected from H, alkyl and aryl;
R⁶, R⁷, R⁸, R⁹ and R¹⁰ are each independently selected from alkyl, aryl and aralkyl; and
G is an optionally substituted C₁-C₆ alkyl group, an optionally substituted aryl group, or an optionally substituted heteroaryl group;
as an intermediate in a process is for preparing a fluorinated molecule, wherein said process comprises a step of converting a carbon-SG bond to a carbon-F bond by treating with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium; and
(ii) a source of fluoride.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

1. A process for incorporating a fluorine atom into a molecule, said process comprising converting a compound of Formula (I) into a compound of Formula (II): 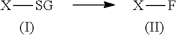
wherein G is an optionally substituted C₁-C₆ alkyl group, an optionally substituted aryl group, or an optionally substituted heteroaryl group, and X is an organic group; and wherein the SG group is attached to a secondary or tertiary carbon atom in the organic group X; said process comprising treating said compound of Formula (I) with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium or chloronium; and
(ii) a source of fluoride.

2. The process according to claim 1 which comprises simultaneously treating the compound of Formula (I) with said activator and said source of fluoride.

3. The process according to any preceding claim, wherein the fluorine is ¹⁸F.

4. The process according to any preceding claim, wherein G is a phenyl or heteroaryl group optionally substituted by one or more substituents independently selected from alkoxy, nitro, halo, and alkyl, or wherein G is selected from methyl and trifluoromethyl.

5. The process according to any preceding claim, wherein G is selected from phenyl, ortho-alkoxyphenyl, para-alkoxyphenyl, and ortho, para-dialkoxyphenyl; preferably wherein G is selected from phenyl and para-methoxyphenyl.

6. The process according to any preceding claim, wherein the activator compound is selected from the group consisting of N-bromosuccinimide, N-chlorosuccinimide, N-fluorobenzenesulfonimide, N-chlorobenzenesulfonimide, N-bromobenzenesulfonimide, diethylaminosulfur trifluoride, diethylaminodifluorosulfinium tetrafluoroborate, morpholinodifluorosulfinium tetrafluoroborate, bromine, PIDA/I₂, AgNO₃/I₂, and AgNO₃/Br₂.

7. The process according to any one of claims 1 to 5 wherein the activator compound is selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium.

8. The process according to any preceding claim, wherein the activator compound is selected from the group consisting of N-bromosuccinimide, N-chlorosuccinimide, N-fluorobenzenesulfonimide, N-chlorobenzenesulfonimide, N-bromobenzenesulfonimide, diethylaminosulfur trifluoride, diethylaminodifluorosulfinium tetrafluoroborate, morpholinodifluorosulfinium tetrafluoroborate, and bromine; and is more preferably, N-bromosuccinimide or N-chlorosuccinimide.

9. The process according to any preceding claim, wherein the source of fluoride is a fluoride phase transfer catalyst.

10. The process according to any one of claims 1 to 8, wherein the source of fluoride is selected from the group consisting of metal fluoride salts, complexes or chelates of hydrogen fluoride or metal fluoride salts, and tetraalkylammonium fluorides.

11. The process according to any one of claims 1 to 8, wherein the source of fluoride is a metal fluoride salt, preferably an alkali metal salt.

12. The process according to any one of claims 1 to 8, wherein the source of fluoride is pyridine-HF or triethylamine-HF.

13. The process according to any one of claims 1 to 8, wherein the source of fluoride is pyridine-HF.

14. The process according to any one of claims 1 to 8, wherein the source of fluoride is a crown ether complex of a metal fluoride, preferably a crown ether complex of an alkali metal fluoride.

15. The process according to any one of claims 1 to 8, wherein the source of fluoride is a C₁-C₄ tetraalkylammonium fluoride, preferably wherein the source of fluoride is tetrabutylammonium fluoride.

16. The process according to any one of claims 1 to 8, wherein the source of fluoride is pyridine-HF and the activator is N-bromosuccinimide.

17. The process according to any one of claims 1 to 8, wherein the activator and the source of fluoride are the same.

18. The process according to claim 17, wherein the activator and the source of fluoride are N-fluorobenzenesulfonimide.

19. The process according to any preceding claim, wherein the process is carried out in an organic solvent, an aqueous solvent, or combinations thereof; preferably wherein the process is carried out in dichloromethane, tetrahydrofuran, dimethylsulfoxide, acetonitrile, benzonitrile, ethanol, methanol, toluene, water, and combinations thereof.

20. The process according to any preceding claim, wherein the process is carried out at room temperature.

21. The process according to any preceding claim wherein the organic group X is of Formula L-Y, where L is a direct bond or a covalent linker group, and Y is selected from the group consisting of a labelling agent, a dye, an amino acid, a peptide, a peptoid, a drug molecule or fragment thereof, an antibody or fragment thereof, a protein, a carbohydrate, a lipid, a nucleobase, a nucleoside, a nucleotide, an oligonucleotide, a polynucleotide, a peptide nucleic acid, and derivatives thereof.

22. The process according to any preceding claim, which process comprises the step of converting a compound of Formula (IIa) to a compound of Formula (Ia): 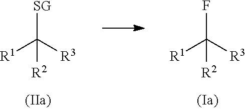
wherein:
R¹ and R² are each independently an optionally substituted hydrocarbyl group;
R³ is H or an optionally substituted hydrocarbyl group;
wherein any two of R¹, R², and R³ or all of R¹, R², and R³ may optionally form one or more cyclic groups.

23. The process according to claim 22 wherein:
R¹ and R² are each independently an optionally substituted aliphatic group; and
R³ is H or an optionally substituted aliphatic group.

24. The process according to claim 22 wherein:
R¹ is an optionally substituted aryl or heteroaryl group;
R² is an optionally substituted aliphatic group; and
R³ is H or an optionally substituted aliphatic group.

25. The process according to claim 22, wherein the carbon to which the R¹, R², R³ and SG groups are bonded in Formula (IIa) is a stereogenic centre and the conversion of the compound of Formula (IIa) to Formula (Ia) results in at least partial inversion of the stereochemical configuration of the corresponding carbon atom to which F is bonded in Formula (Ia) with respect to that of Formula (IIa); preferably complete inversion.

26. The process according to any one of claims 1 to 25 for incorporating a fluorine atom into a peptide, said process comprising the steps of:
(a) incorporating at least one compound of Formula (III) into the backbone of a peptide to form a peptide precursor, 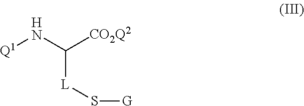
wherein:
Q¹ is H or an amine protecting group;
Q² is H or a carboxyl protecting group;
L is a linker group; and
G is an optionally substituted C₁-C₆ alkyl group, an optionally substituted aryl group, or an optionally substituted heteroaryl group; and
(b) treating the peptide precursor formed in step (a) with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium, or chloronium; and
(ii) a source of fluoride.

27. The process according to claim 26 wherein the activator compound is selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium.

28. The process according to any one of claims 1 to 25 for incorporating a fluorine into a peptoid, said process comprising the steps of:
(a) incorporating at least one compound of Formula (IV) into the backbone of a peptoid to form a peptoid precursor, 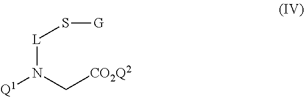
wherein:
Q¹ is H or an amine protecting group;
Q² is H or a carboxyl protecting group;
L is a linker group; and
G is an optionally substituted C₁-C₆ alkyl group, an optionally substituted aryl group, or an optionally substituted heteroaryl group; and
(b) treating the peptoid precursor formed in step (a) with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium, or chloronium; and
(ii) a source of fluoride.

29. The process according to claim 28 wherein the activator compound is selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium.

30. The process according to any one of claims 26 to 29 wherein L is a linker group (CR⁴R⁵), where n is an integer from 1 to 10, and each R⁴ and R⁵ is independently selected from H, alkyl, aryl and COOH, NH₂.

31. The process according to claim 26 or claim 27 wherein said compound of Formula (III) is selected from:

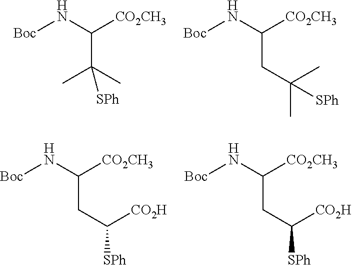

32. The process according to any one of claim 26, 27 or 31, wherein step (a) comprises preparing the peptide precursor by solid phase peptide synthesis.

33. The process according to any one of claims 1 to 25 for incorporating a fluorine atom into an oligo- or poly-nucleotide, the process comprising:
(a) preparing an oligo- or poly-nucleotide precursor comprising at least one —SG group, wherein G is an optionally substituted C₁-C₆ alkyl group, an optionally substituted aryl group, or an optionally substituted heteroaryl group;
(b) treating the oligo- or poly-nucleotide precursor formed in step (a) with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with 12; and other sources of Br⁺, Cl⁺, F⁺, I⁺, bromonium, iodonium, or chloronium; and
(ii) a source of fluoride.

34. The process according to claim 33 wherein the activator compound is selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium.

35. The process according to claim 33 or claim 34 wherein the —SG group is attached via a covalent linker group L to the nucleobase of at least one nucleotide in the oligo- or poly-nucleotide.

36. The process according to any one of claims 33 to 35 wherein the —SG group is incorporated by reacting a compound of Formula (V), 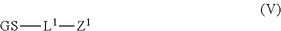
wherein:
Z¹ is a functional group selected from NH₂, N₃, COOH and OH;
L¹ is a linker group (CR⁴R⁵), where n is an integer from 1 to 10;
each R⁴ and R⁵ is independently selected from H, alkyl and aryl; and
G is an optionally substituted C₁-C₆ alkyl group, an optionally substituted aryl group, or an optionally substituted heteroaryl group;
with a reactive group on the nucleobase.

37. The process according to claim 36 wherein the —SG group is incorporated by reacting a compound of Formula (Va), 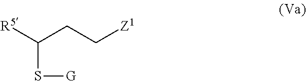
wherein:
Z¹ is a functional group selected from NH₂, N₃, COOH and OH; and
R^{5′} is an aryl group or a tertiary alkyl group;
with a reactive group on the nucleobase.

38. The process according to claim 36 or claim 37 wherein Z¹ is N₃ and the reactive group on the nucleobase is a-C≡CH group.

39. The process according to claim 36 or claim 37 wherein Z¹ is a-C≡CH group and the reactive group on the nucleobase is N₃.

40. The process according to claim 36 or claim 37 wherein the compound of Formula (V) is selected from the following:

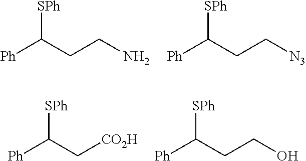

41. The process according to any one of claims 1 to 25 for incorporating a fluorine atom into a molecule, the process comprising:
(a) preparing a precursor molecule comprising at least one —SG group, wherein G is an optionally substituted C₁-C₆ alkyl group, an optionally substituted aryl group, or an optionally substituted heteroaryl group;
(b) treating the precursor molecule formed in step (a) with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium, or chloronium; and
(ii) a source of fluoride.

42. The process according to claim 41 wherein the activator compound is selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium.

43. A process according to claim 41 or claim 42 wherein the —SG group is incorporated into the precursor molecule by reacting a compound of Formula (VI), 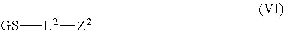
wherein:
Z² is a functional group selected from NH₂, NHR⁶, N₃, COOH, COOR⁷, OH, CN, CONHR⁸, COR⁹, CHO, CSNHR¹⁰, C≡CH, C≡CH₂, and 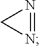
L² is a linker group selected from (CR⁴R⁵) n, an optionally substituted aryl group, an optionally substituted heteroaryl group, and any combination thereof;
wherein n is an integer from 1 to 10;
each R⁴ and R⁵ is independently selected from H, alkyl and aryl;
R⁶, R⁷, R⁸, R⁹, and R¹⁰ are each independently selected from alkyl, aryl and aralkyl; and
G is an optionally substituted C₁-C₆ alkyl group, an optionally substituted aryl group, or an optionally substituted heteroaryl group;
with a reactive group in the molecule.

44. The process according to claim 43 wherein L² is a linker group selected from (CR⁴R⁵)ₙ, an optionally substituted aryl group, and any combination thereof.

45. The process according to claim 43 or claim 44 wherein the —SG group is incorporated into the precursor molecule by reacting a compound of Formula (VIa), 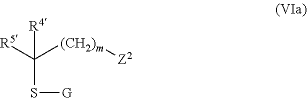
wherein m is an integer from 1 to 10, and
wherein R^{4′} is an aryl group or a tertiary alkyl group and R^{5′} is H, an aryl group or a tertiary alkyl group; or wherein R^{4′} and R^{5′} are both alkyl groups;
with a reactive group in the molecule.

46. The process according to claim 45 wherein the compound of Formula (VIa) is selected from the following:

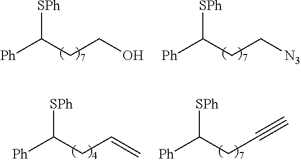

47. The process according to claim 45, wherein m is 1, 2, or 3.

48. The process according to claim 43, claim 45, or claim 47 wherein:
Z² is a functional group selected from NH₂, NHR⁶, N₃, COOH, COOR⁷, OH, CN, CONHR⁸, COR⁹, CHO, CSNHR¹⁰;
L² is a linker group (CR⁴R⁵)ₙ, wherein n is an integer from 1 to 10;
each R⁴ and R⁵ is independently selected from H, alkyl and aryl; and
R⁶, R⁷, R⁸, R⁹, and R¹⁰ are each independently selected from alkyl, aryl and aralkyl.

49. The process according to claim 47 wherein the compound of Formula (VIa) is selected from the following:

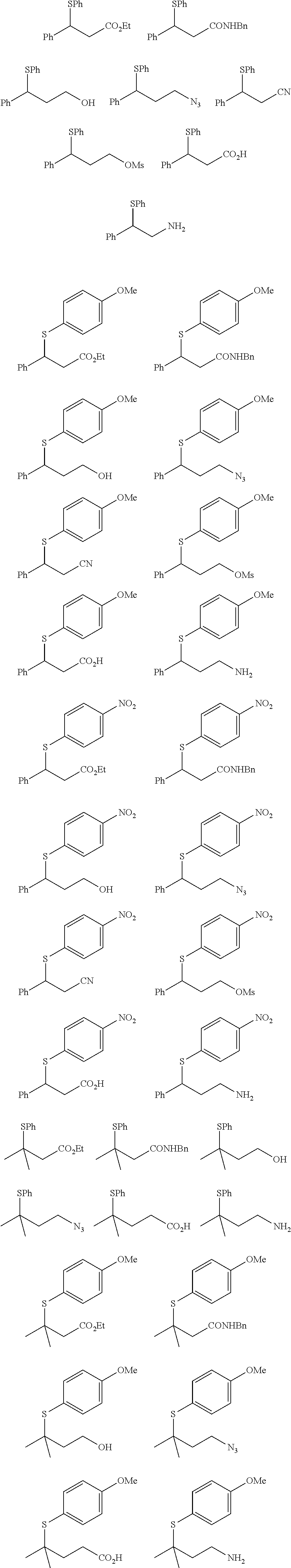

50. The process according to claim 43 wherein the —SG group is incorporated into the precursor molecule by reacting a compound of Formula (VIb): 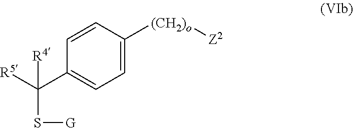
wherein o is 0, 1, 2 or 3, and
wherein R^{4′} is an aryl group or a tertiary alkyl group and R^{5′} is H, an aryl group or a tertiary alkyl group; or wherein R^{4′} and R^{5′} are both alkyl groups;
with a reactive group in the molecule.

51. The process according to claim 50 wherein the compound of Formula (VIb) is selected from the following:

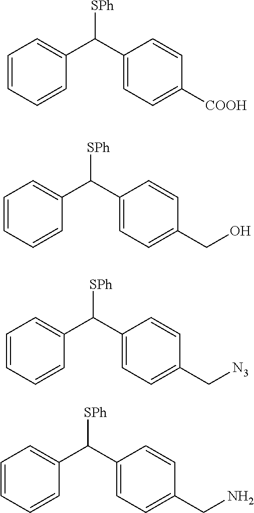

52. The process according to any one of claims 41 to 51 wherein the molecule is selected from the group consisting of nucleosides, nucleotides, oligo- or poly-nucleotides, peptide nucleic acids, amino acids, mono-oligo- and poly-saccharides, peptides, peptoids, proteins and small molecules.

53. Use of a process according to any one of claims 1 to 25 in the preparation of a fluorinated amino acid or a fluorinated peptide.

54. Use of a process according to any one of claims 1 to 25 in the preparation of a fluorinated oligonucleotide or polynucleotide.

55. Use of a process according to any one of claims 1 to 25 in the preparation of a fluorinated pharmacologically active agent, or a fragment thereof, or a precursor or intermediate thereof.

56. Use of a process according to any one of claims 1 to 25 in the preparation of a fluorinated radiolabelling agent.

57. Use of a compound of Formula (III) 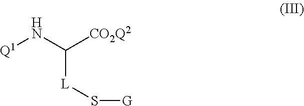
wherein:
Q¹ is H or an amine protecting group;
Q² is H or a carboxyl protecting group;
L is a linker group (CR⁴R⁵), where n is an integer from 1 to 10;
each R⁴ and R⁵ is independently selected from H, alkyl, aryl, COOH, and NH₂; and
G is an optionally substituted C₁-C₆ alkyl group, an optionally substituted aryl group, or an optionally substituted heteroaryl group;
as an intermediate in a process is for preparing fluorinated peptides, wherein said process comprises a step of converting a carbon-SG bond to a carbon-F bond by treating with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium, or chloronium; and
(ii) a source of fluoride.

58. The use according to claim 57 wherein the activator compound is selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium.

59. Use of a compound of Formula (IV) 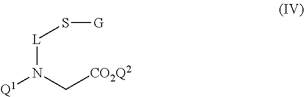
wherein:
Q¹ is H or an amine protecting group;
Q² is H or a carboxyl protecting group;
L is a linker group; and
G is an optionally substituted C₁-C₆ alkyl group, an optionally substituted aryl group, or an optionally substituted heteroaryl group;
as an intermediate in a process is for preparing fluorinated peptoids, wherein said process comprises a step of converting a carbon-SG bond to a carbon-F bond by treating with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium, or chloronium; and
(ii) a source of fluoride.

60. The use according to claim 59 wherein the activator compound is selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium.

61. Use of a compound of Formula (V), 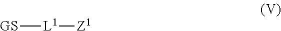
wherein:
Z¹ is a functional group selected from NH₂, N₃, COOH and OH;
L¹ is a linker group (CR⁴R⁵), where n is an integer from 1 to 10;
each R⁴ and R⁵ is independently selected from H, alkyl and aryl; and
G is an optionally substituted C₁-C₆ alkyl group, an optionally substituted aryl group, or an optionally substituted heteroaryl group;
as an intermediate in a process is for preparing a fluorinated oligo- or poly-nucleotide, wherein said process comprises a step of converting a carbon-SG bond to a carbon-F bond by treating with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium, or chloronium; and
(ii) a source of fluoride.

62. The use according to claim 61 wherein the activator compound is selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium.

63. Use of a compound of Formula (VI), 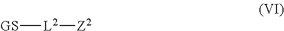
wherein:
Z² is a functional group selected from NH₂, NHR⁶, N₃, COOH, COOR⁷, OH, CN, CONHR⁸, COR⁹, CHO, CSNHR¹⁰, C≡CH, C≡CH₂, and 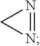
L² is a linker group selected from (CR⁴R⁵)ₙ, an optionally substituted aryl group, an optionally substituted heteroaryl group, an optionally substituted cycloalkyl group, and any combination thereof;
wherein n is an integer from 1 to 10;
each R⁴ and R⁵ is independently selected from H, alkyl and aryl;
R⁶, R⁷, R⁸, R⁹, and R¹⁰ are each independently selected from alkyl, aryl and aralkyl; and
G is an optionally substituted C₁-C₆ alkyl group, an optionally substituted aryl group, or an optionally substituted heteroaryl group;
as an intermediate in a process is for preparing a fluorinated molecule, wherein said process comprises a step of converting a carbon-SG bond to a carbon-F bond by treating with:
(i) an activator compound selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine, hypervalent iodine compounds with I₂; and other sources of Br⁺, Cl⁺, F⁺, I⁺, bromonium, iodonium, or chloronium; and
(ii) a source of fluoride.

64. The use according to claim 63 wherein:
Z² is a functional group selected from NH₂, NHR⁶, N₃, COOH, COOR⁷, OH, CN and CONHR⁸, COR⁹, CHO, and CSNHR¹⁰;
L² is a linker group (CR⁴R⁵), where n is an integer from 1 to 10;
each R⁴ and R⁵ is independently selected from H, alkyl and aryl;
R⁶, R⁷, R⁸, R⁹, and R¹⁰ are each independently selected from alkyl, aryl and aralkyl; and
wherein the activator compound is selected from the group consisting of N-halosuccinimides, N-halobenzenesulfonimides, N-halobenzenesulfonamides, dialkylaminodihalosulfinium salts, heterocyclylaminodihalosulfinium salts, dialkylaminosulfur trihalides, XeF₂, difluoroiodotoluene, di- and tri-bromoisocyanuric acids, bromine, chlorine; and other sources of Br⁺, Cl⁺, F⁺, bromonium or chloronium.
